(19) United States
(12) Patent Application Publication
(10) Pub. No.: US 2021/0321942 A1
(43) Pub. Date: Oct. 21, 2021

(54) SYSTEMS AND METHODS FOR BIOMONITORING AND PROVIDING PERSONALIZED HEALTHCARE

(71) Applicant: INFORMED DATA SYSTEMS INC. D/B/A ONE DROP, New York, NY (US)

(72) Inventors: Ashwin Pushpala, San Francisco, CA (US); Matthew Chapman, Oakland, CA (US); Ydo Wexler, Haifa (IL); Daniel R. Goldner, Minnetonka, MN (US); Jeffrey Dachis, Brooklyn, NY (US)

(21) Appl. No.: 17/236,753

(22) Filed: Apr. 21, 2021

Related U.S. Application Data
(60) Provisional application No. 63/150,069, filed on Feb. 16, 2021.
(60) Provisional application No. 63/108,198, filed on Oct. 30, 2020.
(60) Provisional application No. 63/032,415, filed on May 29, 2020.
(60) Provisional application No. 63/013,388, filed on Apr. 21, 2020.

(51) Int. Cl. A61B 5/00; A61B 5/145; A61B 5/01; A61B 5/024; A61B 5/28

(52) U.S. Cl. CPC A61B 5/6833; A61B 5/685; A61B 5/14546; A61B 5/0004; A61B 5/14532; A61B 2562/0233; A61B 5/02416; A61B 5/28; A61B 2562/0271; A61B 2562/0219; A61B 5/01

(57) ABSTRACT

Systems, devices, and methods for biomonitoring are disclosed herein. In some embodiments, a device for monitoring a user's health comprises a patch including a substrate configured to couple to the user's skin, and an array of microneedles carried by the substrate. The array of microneedles can be configured to access interstitial fluid in the user's skin and generate a first electrical signal indicative of at least one analyte in the interstitial fluid. The device can include a pod configured to releasably couple to the patch, the pod having at least one sensor configured to generate a second electrical signal indicative of a physiological parameter of the user. The pod can further include a processor configured to receive and process the first and second electrical signals to generate health measurements for the user. The pod can also include a communication unit configured to transmit the health measurements to a remote device.

### CROSS-REFERENCE TO RELATED APPLICATIONS

The present application claims the benefit of: U.S. Provisional Patent Application No. 63/013,388, filed Apr. 21, 2020, entitled SYSTEMS AND METHODS FOR BIOMONITORING AND PROVIDING PERSONALIZED HEALTHCARE; U.S. Provisional Patent Application No. 63/032,415, filed May 29, 2020, entitled SYSTEMS AND METHODS FOR BIOMONITORING AND PROVIDING PERSONALIZED HEALTHCARE; U.S. Provisional Patent Application No. 63/108,198, filed Oct. 30, 2020, entitled SYSTEMS AND METHODS FOR BIOMONITORING AND PROVIDING PERSONALIZED HEALTHCARE; and U.S. Provisional Patent Application No. 63/150,069, filed Feb. 16, 2021, entitled MULTI-ANALYTE PATCH SENSOR AND ASSOCIATED SYSTEMS AND METHODS, all of which are incorporated by reference herein in their entireties.

The following commonly assigned U.S. Patent Applications and U.S. Patents are incorporated herein by reference in their entireties:

U.S. Pat. No. 10,173,042, filed Dec. 15, 2016, entitled METHOD OF MANUFACTURING A SENSOR FOR SENSING ANALYTES;

U.S. Pat. No. 10,820,860, filed Jan. 19, 2017, entitled ON-BODY MICROSENSOR FOR BIOMONITORING;

U.S. Pat. No. 10,595,754, filed May 22, 2017, entitled SYSTEM FOR MONITORING BODY CHEMISTRY;

U.S. Patent Application Publication No. 2018/0140235, filed Jan. 22, 2018, entitled SYSTEM FOR MONITORING BODY CHEMISTRY;

U.S. Patent Application Publication No. 2020/0077931, filed Sep. 3, 2019, entitled FORECASTING BLOOD GLUCOSE CONCENTRATION;

U.S. Patent Application Publication No. 2020/0375549, filed May 29, 2020, entitled SYSTEMS FOR BIOMONITORING AND BLOOD GLUCOSE FORECASTING, AND ASSOCIATED METHODS; and

U.S. patent application Ser. No. 17/167,795, filed Feb. 4, 2021, entitled FORECASTING AND EXPLAINING USER HEALTH METRICS.

### TECHNICAL FIELD

This disclosure relates generally to medical devices and, in particular, to sensors for biomonitoring and associated systems and methods.

### BACKGROUND

Many individuals suffer from chronic health conditions, such as diabetes, pre-diabetes, hypertension, or hyperlipidemia. For example, diabetes mellitus (DM) is a group of metabolic disorders characterized by high blood glucose levels over a prolonged period. Typical symptoms of such conditions include frequent urination, increased thirst, increased hunger, etc. If left untreated, diabetes can cause many complications. There are three main types of diabetes: Type 1 diabetes, Type 2 diabetes, and gestational diabetes. Type 1 diabetes results from the pancreas' failure to produce enough insulin. In Type 2 diabetes, cells fail to respond to insulin properly. Gestational diabetes occurs when pregnant women without a previous history of diabetes develop high blood glucose levels.

Timely and proper diagnoses and treatment are essential to maintaining a relatively healthy lifestyle for individuals with chronic health conditions. For example, diabetes treatment typically relies on accurate determination of glucose concentration in the blood of an individual at a present time and/or in the future. However, conventional blood glucose monitoring systems may be unable to provide real-time analytics, personalized analytics, or blood glucose concentration forecasting, or may not provide such information in a rapid, reliable, and accurate manner. Additionally, conventional systems may not be capable of monitoring and forecasting health parameters for other types of chronic health conditions. Thus, there is a need for improved systems and methods for biomonitoring and/or providing personalized healthcare recommendations or information for the treatment of diabetes and other chronic health conditions.

### BRIEF DESCRIPTION OF THE DRAWINGS

FIG. 1 is a schematic diagram of a computing environment in which a biomonitoring and healthcare guidance system operates, in accordance with embodiments of the present technology.

FIG. 2 is a schematic illustration of a biosensor device configured in accordance with embodiments of the present technology.

FIGS. 3A-3R illustrate a representative example of a biosensor device configured in accordance with embodiments of the present technology.

FIGS. 4A-4C illustrate an applicator configured in accordance with embodiments of the present technology.

FIGS. 5A and 5B illustrate the applicator of FIGS. 4A-4C together with a pedestal configured in accordance with embodiments of the present technology.

FIG. 5C illustrates the biosensor device of FIGS. 3A-3R on the pedestal of FIGS. 5A and 5B.

FIGS. 5D-5F illustrate the biosensor device of FIGS. 3A-3R, the applicator of FIGS. 4A-4C, and the pedestal of FIGS. 5A-5C during operation, in accordance with embodiments of the present technology.

FIGS. 6A-6C illustrate a charging station configured in accordance with embodiments of the present technology.

FIG. 6D illustrates the charging station of FIGS. 6A-6C together with the applicator of FIGS. 4A-4C and the pedestal of FIGS. 5A-5C.

FIGS. 7A-7D are partially schematic illustrations of microneedles configured in accordance with embodiments of the present technology.

FIG. 8 is a schematic illustration of a biosensor including multiple microneedle arrays in accordance with embodiments of the present technology.

FIGS. 9A-9D are schematic illustrations of various stages of a method or process for manufacturing microneedle arrays, in accordance with embodiments of the present technology.

FIG. 10 is a block diagram illustrating a method for generating a prediction or forecast of a user's health parameters, in accordance with embodiments of the present technology.

FIG. 11 is a block diagram illustrating a method for forecasting or predicting a health state of a user, in accordance with embodiments of the present technology.

FIG. 12 is a block diagram illustrating a method for forecasting a health parameter of a user, in accordance with embodiments of the present technology.

FIGS. 13A-13N illustrate various examples of user interfaces configured in accordance with embodiments of the present technology.

FIG. 14 is a schematic block diagram of a computing system or device configured in accordance with embodiments of the present technology.

### DETAILED DESCRIPTION

The present technology generally relates to systems, devices, and methods for biomonitoring and providing personalized healthcare guidance. In some embodiments, a biosensor for monitoring a user's health includes a patch including a substrate configured to couple to the user's skin, and an array of microneedles carried by the substrate. The array of microneedles can be configured to access interstitial fluid in the user's skin and generate a first electrical signal indicative of at least one analyte in the interstitial fluid. The device can include a pod configured to releasably couple to the patch, the pod having at least one sensor configured to generate a second electrical signal indicative of a physiological parameter of the user. The pod can further include a processor configured to receive and process the first electrical signal to generate a first health measurement for the user, and receive and process the second electrical signal to generate a second health measurement for the user. The pod can also include a communication unit configured to transmit the health measurements to a remote device. The health measurements can be input into one or more machine learning models to generate predictions of the user's future health state. The predictions can be used to determine personalized healthcare guidance, such as recommendations for monitoring and/or managing a disease or condition, or otherwise maintaining or improving user health.

In some embodiments, a biosensor configured in accordance with the present technology can detect and generate measurements of multiple different health parameters. Such biosensors may be referred to herein as “multi-analyte” or “multi-parameter” sensors. The multi-analyte sensor technology described herein allows for more accurate predictions, e.g., compared to predictions generated based on data from single-analyte sensors. For instance, aggregation of multiple health parameter measurements into a single data stream and/or database can improve accuracy when generating predictions of an individual health parameters, since the prediction model has more context of the user's health state. Additionally, the multi-analyte sensor technology can allow for predictions of more complex diseases, conditions, and/or health states than would be possible using single-analyte sensors.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

The headings provided herein are for convenience only and do not interpret the scope or meaning of the claimed present technology.

### I. SYSTEMS FOR BIOMONITORING AND HEALTHCARE GUIDANCE

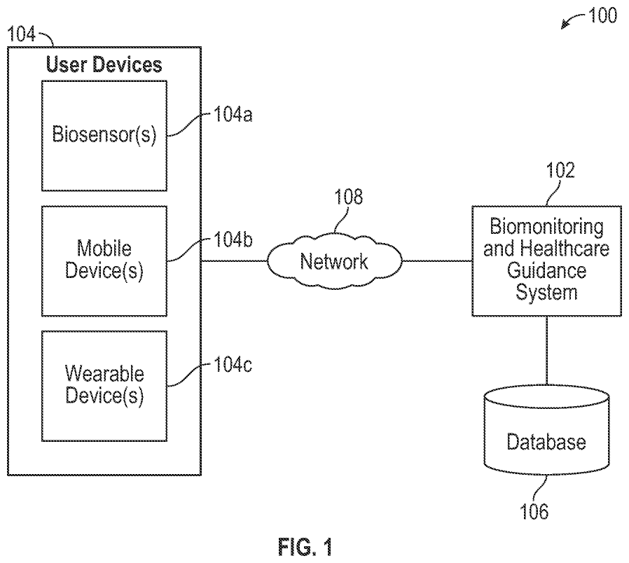

FIG. 1 is a schematic diagram of a computing environment 100 in which a biomonitoring and healthcare guidance system 102 (“system 102”) operates, in accordance with embodiments of the present technology. As shown in FIG. 1, the system 102 is operably coupled to one or more user devices 104 via a network 108. The system 102 is also operably coupled to at least one database or storage component 106 (“database 106”). The system 102 can include processors, memory, and/or other software and/or hardware components configured to implement the various methods described herein. For example, the system 102 can be configured to monitor a user's health state and provide information to support personalized healthcare, as described in greater detail below.

The health state can be any status, condition, parameter, etc., that is associated with or otherwise related to the user's health. In some embodiments, the system 102 receives input data and performs monitoring, processing, analysis, forecasting, interpretation, etc., of the input data in order to generate instructions, notifications, recommendations, support, and/or other information to the user that may be useful for self-care of diseases or conditions, such as chronic conditions (e.g., diabetes (type 1 and type 2), pre-diabetes, hypertension, hyperlipidemia, etc.), acute conditions, etc. For example, the system 102 can be used to identify, manage, and/or monitor a variety of different diseases, conditions, and/or other health states, including, but not limited to: diabetes and associated conditions (e.g., hypoglycemia, hyperglycemia, ketoacidosis), liver diseases (e.g., hepatitis A, hepatitis B, hepatitis C, fatty liver disease, cirrhosis, liver failure), cardiovascular diseases (e.g., congestive heart failure, coronary artery disease, peripheral vascular disease, hypertension, arrhythmia, cardiomyopathy), cancer (e.g., bladder cancer, breast cancer, colorectal cancer, endometrial cancer, kidney cancer, leukemia, liver cancer, lung cancer, skin cancer, lymphoma, pancreatic cancer, prostate cancer, thyroid cancer), lung diseases (e.g., asthma, chronic obstructive pulmonary disease, hypoxia, bronchitis, cystic fibrosis), kidney diseases (e.g., chronic kidney disease), brain conditions (e.g., acute brain conditions, chronic brain conditions), ophthalmological diseases, intoxication, dehydration, hyponatremia, shock, heat stroke, infection, sepsis, trauma, water retention, bleeding, endocrine disorders, muscle breakdown, malnutrition, body function (e.g., lung functions, heart functions, kidney functions, thyroid functions, adrenal functions, etc.), women's health (e.g., gynecological diseases and conditions such as polycystic ovary syndrome (PCOS), pregnancy, fertility), drug use (e.g., smoking, alcohol, or other drugs), physical performance (e.g., athletic performance), anaerobic activity, weight loss or gain, obesity, nutrition, eating disorders, metabolism (e.g., lipid metabolism, protein metabolism, aerobic metabolism), wellness, mental health, focus, stress, effects of medication, medication levels, health indicators, and/or user compliance. For example, the embodiments herein can be used to diagnose, monitor, track, and/or provide digital therapy using behavior change, drug or therapy titration, risk assessment, or the like.

The input data for the system 102 can include health-related information, contextual information, and/or any other information relevant to the user's health state. For example, health-related information can include levels or concentrations of a biomarker, such as glucose, gases (e.g. oxygen, carbon dioxide, etc.), electrolytes (e.g., bicarbonate, potassium, sodium, magnesium, chloride, lactic acid), blood urea nitrogen (BUN), creatinine, ketones, cholesterol, triglycerides, alcohols, amino acids, neurotransmitters, hormones, disease biomarkers (e.g., cancer biomarkers, cardiovascular disease biomarkers), drugs, pH, cell count, and/or other biomarkers. Health-related information can also include physiological and/or behavioral parameters, such as vitals (e.g., heart rate, body temperature (such as skin temperature), blood pressure (such as systolic and/or diastolic blood pressure), respiratory rate), cardiovascular data (e.g., pacemaker data, arrhythmia data), body function data, meal or nutrition data (e.g., number of meals; timing of meals; number of calories; amount of carbohydrates, fats, sugars, etc.), physical activity or exercise data (e.g., time and/or duration of activity; activity type such as walking, running, swimming; strenuousness of the activity such as low, moderate, high; etc.), sleep data (e.g., number of hours of sleep, average hours of sleep, variability of hours of sleep, sleep-wake cycle data, data related to sleep apnea events, sleep fragmentation (such as fraction of nighttime hours awake between sleep episodes, etc.)), stress level data (e.g., cortisol and/or other chemical indicators of stress levels, perspiration), a1c data, etc. Health-related information can also include medical history data (e.g., weight, age, sleeping patterns, medical conditions, cholesterol levels, triglyceride levels, disease type, family history, user health history, diagnoses, tobacco usage, alcohol usage, etc.), diagnostic data (e.g., molecular diagnostics, imaging), medication data (e.g., timing and/or dosages of medications such as insulin), personal data (e.g., name, gender, demographics, social network information, etc.), and/or any other data, and/or any combination thereof. Contextual information can include user location (e.g., GPS coordinates, elevation data), environmental conditions (e.g., air pressure, humidity, temperature, air quality, etc.), and/or combinations thereof.

Table 1 below lists examples of health parameters and associated diseases, conditions, and/or health states. The systems and devices described herein can be configured to monitor any of the health parameters listed in Table 1.

TABLE 1
Representative Health Parameters for Biomonitoring

| Health | |
|---|---|
| Parameter | Disease/Condition/Health State |
| Glucose | Diabetes, Weight Loss, Athletic Performance, |
| | Nutrition, Wellness, Focus |
| Oxygen | Hypoxia, Athletic Performance, Cardiac Health, Lung |
| | Function |
| Potassium | Dehydration, Cardiac Health, Diabetes, Kidney |
| | Disease, Blood Pressure |
| Sodium | Dehydration, Acute/Chronic Brain Conditions, Lung |
| | Function, Liver Function, Cardiac Health, Kidney |
| | Health, Thyroid, Adrenal |
| Lactic Acid | Shock, Sepsis, Anaerobic Activity, Metabolism, Liver |
| | Failure, Diabetic Ketoacidosis, Drugs/Toxins, Kidney |
| | Disease, Hypoxia |
| Urea/BUN | Kidney Disease, Sepsis, Hypoxia, Protein Metabolism, |
| | Nutrition |
| Ketones | Diabetic Ketoacidosis, Nutrition, Weight Loss, Lipid |
| | Metabolism |
| Bicarbonate | Kidney Disease, Liver Disease, Lung Disorders, Blood |
| | Pressure, Aerobic Metabolism |
| Temperature | Infection, Fertility, Metabolism, Athletic Performance, |
| | Heat Stroke |
| Heart Rate | Cardiac Health, Metabolism, Athletic Performance, |
| | Weight Loss, Stress |

In some embodiments, the system 102 receives input data from one or more user devices 104. The user devices 104 can be any device associated with a user (e.g., a patient), and can be used to obtain healthcare information, contextual information, and/or any other relevant information relating to the user and/or any other users (e.g., appropriately anonymized user data). In the illustrated embodiment, for example, the user devices 104 include at least one biosensor 104a (e.g., blood glucose sensors, pressure sensors, heart rate sensors, sleep trackers, temperature sensors, motion sensors, or other biomonitoring devices), at least one mobile device 104b (e.g., a smartphone or tablet computer), and, optionally, at least one wearable device 104c (e.g., a smartwatch, fitness tracker). In other embodiments, however, one or more of the user devices 104a-c can be omitted and/or other types of user devices can be included, such as computing devices (e.g., personal computers, laptop computers, etc.). Moreover, although FIG. 1 illustrates the biosensor(s) 104a as being separate from the other user devices 104, in other embodiments the biosensor(s) 104a can be incorporated into another user device 104. Additional examples of biosensors 104a suitable for use with the present technology are described in greater detail below.

In some embodiments, some or all of the user devices 104 are configured to periodically or continuously obtain any of the above data (e.g., health-related information and/or contextual information) from the user over a particular time period (e.g., hours, days, weeks, months, years). For example, data can be obtained at a predetermined time interval (e.g., once every minute, 2 minutes, 5 minutes, 10 minutes, 15 minutes, 20 minutes, 30 minutes, 60 minutes, 2 hours, etc.), at random time intervals, or combinations thereof. The time interval for data collection can be set by the user, by another user (e.g., a physician), by the system 102, or by the user device 104 itself (e.g., as part of an automated data collection program). The user device 104 can obtain the data automatically or semi-automatically (e.g., by automatically prompting the user to provide such data at a particular time), or from manual input by the user (e.g., without prompts from the user device 104). The continuous data may be provided to the system 102 at predetermined time intervals (e.g., once every minute, 2 minutes, 5 minutes, 10 minutes, 15 minutes, 20 minutes, 30 minutes, 60 minutes, 2 hours, etc.), continuously, in real-time, upon receiving a query, manually, automatically (e.g., upon detection of new data), semi-automatically, etc. The time interval at which the user device 104 obtains data may or may not be the same as the time interval at which the user device 104 transmits the data to the system 102.

The user devices 104 can obtain any of the above data and can provide output in various ways, such as using one or more of the following components: a microphone (e.g., either a separate microphone or a microphone embedded in the device), a speaker, a screen (e.g., using a touchscreen, a stylus pen, and/or in any other fashion), a keyboard, a mouse, a camera, a camcorder, a telephone, a smartphone, a tablet computer, a personal computer, a laptop computer, a sensor (e.g., a sensor included in or operably coupled to the user device 104), and/or any other device. The data obtained by the user devices 104 can include metadata, structured content data, unstructured content data, embedded data, nested data, hard disk data, memory card data, cellular telephone memory data, smartphone memory data, main memory images and/or data, forensic containers, zip files, files, memory images, and/or any other data/information. The data can be in various formats, such as text, numerical, alpha-numerical, hierarchically arranged data, table data, email messages, text files, video, audio, graphics, etc. Optionally, any of the above data can be filtered, smoothed, augmented, annotated, or otherwise processed (e.g., by the user devices 104 and/or the system 102) before being used.

In some embodiments, any of the above data can be queried by one or more of the user devices 104 from one or more databases (e.g., the database 106, a third-party database, etc.). The user device 104 can generate a query and transmit the query to the system 102, which can determine which database may contain requisite information and then connect with that database to execute a query and retrieve appropriate information. In other embodiments, the user device 104 can receive the data directly from the third-party database and transmit the received data to the system 102, or can instruct the third-party database to transmit the data to the system 102. In some embodiments, the system 102 can include various application programming interfaces (APIs) and/or communication interfaces that can allow interfacing between user devices 104, databases, and/or any other components.

Optionally, the system 102 can also obtain any of the above data from various third-party sources, e.g., with or without a query initiated by a user device 104. In some embodiments, the system 102 can be communicatively coupled to various public and/or private databases that can store various information, such as census information, health statistics (e.g., appropriately anonymized), demographic information, population information, and/or any other information. Additionally, the system 102 can also execute a query or other command to obtain data from the user devices 104 and/or access data stored in the database 106. The data can include data related to the particular user and/or a plurality of other users (e.g., health-related information, contextual information, etc.) as described herein.

The database 106 can be used to store various types of data obtained and/or used by the system 102. For example, any of the above data can be stored in the database 106. The database 106 can also be used to store data generated by the system 102, such as previous predictions or forecasts produced by the system 102. In some embodiments, the database 106 includes data for multiple users, such as at least 50, 100, 200, 500, 1000, 2000, 3000, 4000, 5000, or 10,000 different users. The data can be appropriately anonymized to ensure compliance with various privacy standards. The database 106 can store information in various formats, such as table format, column-row format, key-value format, etc. (e.g., each key can be indicative of various attributes associated with the user and each corresponding value can be indicative of the attribute's value (e.g., measurement, time, etc.)). In some embodiments, the database 106 can store a plurality of tables that can be accessed through queries generated by the system 102 and/or the user devices 104. The tables can store different types of information (e.g., one table can store blood glucose measurement data, another table can store user health data, etc.), where one table can be updated as a result of an update to another table.

For example, Table 2 below illustrates exemplary health and/or behavioral data that may be provided to the system 102 and/or stored in the database 106. The data in Table 2 can be generated by one or more user devices 104, as previously described. Each entry in Table 2 is labeled with a user ID, and includes a time stamp indicating when the data was obtained, the type of data, and the data value.

TABLE 2
Health and Behavioral Data

| User ID | Time | Data Type | Value |
|---|---|---|---|
| user1 | 2018 08 30 7:48:15.124 utc | blood glucose | 135 mg/dL |
| user2 | 2018 08 30 7:48:15.126 utc | carbohydrates | 38 g |
| user3 | 2018 08 30 7:48:16.324 utc | activity | 30 min |
| user2 | 2018 08 30 7:48:17.128 utc | medicine: insulin | 6 U |
| user4 | 2018 08 30 7:48:15.226 utc | blood glucose | 218 mg/dL |
| user1 | 2018 08 30 7:48:15.829 utc | carbohydrates | 14 g |
| user5 | 2018 08 30 7:48:17.155 utc | a1c | 7.80% |

As another example, Table 3 below illustrates exemplary personal data that may be provided to the system 102 and/or stored in the database 106. The data in Table 3 can be generated by one or more user devices 104, as previously described. Each entry in Table 3 is labeled with a user ID, and includes personal information for that particular user such as the time zone in which the user is located, the type of diabetes the user has, the date that the user was first enrolled in the system 102, the year in which the user was diagnosed with diabetes, and the user's gender.

TABLE 3
Personal Data

| | | Diabetes | | Diagnosis | |
|---|---|---|---|---|---|
| User ID | Time Zone | Type | Start Date | Year | Gender |
| user1 | New York | Type 2 | 2014 Mar. 5 | 2002 | F |
| user2 | Los Angeles | Type 1 | 2016 Dec. 26 | None | M |
| user3 | Mumbai | Type 2 | 2015 Apr. 8 | 2015 | None |
| user4 | Lisbon | Type 2 | 2017 Sep. 13 | None | M |

In some embodiments, one or more users can access the system 102 via the user devices 104, e.g., to send data to the system 102 (e.g., health-related information and/or contextual information) and/or receive data from the system 102 (e.g., predictions, notifications, recommendations, instructions, support, etc.). The users can be individual users (e.g., patients, healthcare professionals, etc.), computing devices, software applications, objects, functions, and/or any other types of users and/or any combination thereof. For example, upon obtaining any of the input data discussed above, the user device 104 can generate an instruction and/or command to the system 102, e.g., to process the obtained data, store the data in the database 106, extract additional data from one or more databases, and/or perform analysis of the data. The instruction/command can be in a form of a query, a function call, and/or any other type of instruction/command. In some implementations, the instructions/commands can be provided using a microphone (either a separate microphone or a microphone imbedded in the user device 104), a speaker, a screen (e.g., using a touchscreen, a stylus pen, and/or in any other fashion), a keyboard, a mouse, a camera, a camcorder, a telephone, a smartphone, a tablet computer, a personal computer, a laptop computer, and/or using any other device. The user device 104 can also instruct the system 102 to perform an analysis of data stored in the database 106 and/or inputted via the user device 104.

As discussed further below, the system 102 can analyze the obtained input data, including historical data, current real-time data, continuously supplied data, calibration data, and/or any other data (e.g., using a statistical analysis, machine learning analysis, etc.), and generate output data. The output data can include predictions of a user's health state, correlations between data, interpretations, recommendations, notifications, instructions, support, and/or other information related to the obtained input data. In some embodiments, the output data provides information to assist the user in adjusting their behavior (e.g., diet, exercise, sleeping, etc.) to enhance outcomes, to reduce, limit, or avoid health care provider intervention, etc.

The system 102 can perform such analyses at any suitable frequency and/or any suitable number of times (e.g., once, multiple times, on a continuous basis, etc.). For example, when updated input data is supplied to the system 102 (e.g., from the user devices 104), the system 102 can reassess and update its previous output data, if appropriate. In performing its analysis, the system 102 can also generate additional queries to obtain further information (e.g., from the user devices 104, the database 106, or third party sources). In some embodiments, the user device 104 can automatically supply the system 102 with such information. Receipt of updated and/or additional information can automatically trigger the system 102 to execute a process for reanalyzing, reassessing, or otherwise updating previous output data.

In some embodiments, the system 102 is configured to analyze the input data and generate the output data using one or more machine learning models. The machine learning models can include supervised learning models, unsupervised learning models, semi-supervised learning models, and/or reinforcement learning models. Examples of machine learning models suitable for use with the present technology include, but are not limited to: regression algorithms (e.g., ordinary least squares regression, linear regression, logistic regression, stepwise regression, multivariate adaptive regression splines, locally estimated scatterplot smoothing), instance-based algorithms (e.g., k-nearest neighbor, learning vector quantization, self-organizing map, locally weighted learning, support vector machines), regularization algorithms (e.g., ridge regression, least absolute shrinkage and selection operator, elastic net, least-angle regression), decision tree algorithms (e.g., classification and regression trees, Iterative Dichotomiser 3 (ID3), C4.5, C5.0, chi-squared automatic interaction detection, decision stump, M5, conditional decision trees), Bayesian algorithms (e.g., naïve Bayes, Gaussian naïve Bayes, multinomial naïve Bayes, averaged one-dependence estimators, Bayesian belief networks, Bayesian networks), clustering algorithms (e.g., k-means, k-medians, expectation maximization, hierarchical clustering), association rule learning algorithms (e.g., apriori algorithm, ECLAT algorithm), artificial neural networks (e.g., perceptron, multilayer perceptrons, back-propagation, stochastic gradient descent, Hopfield networks, radial basis function networks), deep learning algorithms (e.g., convolutional neural networks, recurrent neural networks, long short-term memory networks, stacked auto-encoders, deep Boltzmann machines, deep belief networks), dimensionality reduction algorithms (e.g., principle component analysis, principle component regression, partial least squares regression, Sammon mapping, multidimensional scaling, projection pursuit, discriminant analysis), time series forecasting algorithms (e.g., exponential smoothing, autoregressive models, autoregressive with exogenous input (ARX) models, autoregressive moving average (ARMA) models, autoregressive moving average with exogenous inputs (ARMAX) models, autoregressive integrated moving average (ARIMA) models, autoregressive conditional heteroskedasticity (ARCH) models), and ensemble algorithms (e.g., boosting, bootstrapped aggregation, AdaBoost, blending, stacking, gradient boosting machines, gradient boosted trees, random forest).

Although FIG. 1 illustrates a single set of user devices 104, it will be appreciated that the system 102 can be operably and communicably coupled to multiple sets of user devices, each set being associated with a particular user or user. Accordingly, the system 102 can be configured to receive and analyze data from a large number of users (e.g., at least 50, 100, 200, 500, 1000, 2000, 3000, 4000, 5000, or 10,000 different users) over an extended time period (e.g., weeks, months, years). The data from these users can be used to train and/or refine one or more machine learning models implemented by the system 102, as described below.

The system 102 and user devices 104 can be operably and communicatively coupled to each other via the network 108. The network 108 can be or include one or more communications networks, and can include at least one of the following: a wired network, a wireless network, a metropolitan area network (“MAN”), a local area network (“LAN”), a wide area network (“WAN”), a virtual local area network (“VLAN”), an internet, an extranet, an intranet, and/or any other type of network and/or any combination thereof. Additionally, although FIG. 1 illustrates the system 102 as being directly connected to the database 106 without the network 108, in other embodiments the system 102 can be indirectly connected to the database 106 via the network 108. Moreover, in other embodiments one or more of the user devices 104 can be configured to communicate directly with the system 102 and/or database 106, rather than communicating with these components via the network 108.

The various components 102-108 illustrated in FIG. 1 can include any suitable combination of hardware and/or software. In some embodiment, components 102-108 can be disposed on one or more computing devices, such as, server(s), database(s), personal computer(s), laptop(s), cellular telephone(s), smartphone(s), tablet computer(s), and/or any other computing devices and/or any combination thereof. In some embodiments, the components 102-108 can be disposed on a single computing device and/or can be part of a single communications network. Alternatively, the components can be located on distinct and separate computing devices. For example, although FIG. 1 illustrates the system 102 as being a single component, in other embodiments the system 102 can be implemented across a plurality of different hardware components at different locations.

### II. BIOSENSORS AND ASSOCIATED DEVICES, KITS, AND METHODS

The systems and methods of the present technology can use one or more biosensors (also referred to herein as “biosensor devices” “sensors,” or “sensor devices”) to generate user data, such as data indicative of a user's health state. The biosensors described herein can be or include various types of sensors, such as chemical sensors, electrochemical sensors, optical sensors (e.g., optical enzymatic sensors, opto-chemical sensors, fluorescence-based sensors, etc.), spectrophotometric sensors, spectroscopic sensors, polarimetric sensors, calorimetric sensors, iontophoretic sensors, radiometric sensors, and the like, and combinations thereof. The biosensors can be implanted sensors, non-implanted sensors, invasive sensors, minimally invasive sensors, non-invasive sensors, wearable sensors, etc. The biosensors can be disposable sensors, reusable sensors, or can include any suitable combination of disposable and reusable components (e.g., a disposable sensor portion for monitoring specific condition(s) and a reusable electronics portion for receiving and processing the sensor data).

The number, configuration, and/or functionality of the biosensors can be selected based on desired sensing capabilities. For example, the biosensors described herein can be configured to sense any suitable combinations of the following health parameters: glucose, gases (e.g. oxygen, carbon dioxide, etc.), electrolytes (e.g., bicarbonate, potassium, sodium, magnesium, chloride, lactic acid), BUN, creatinine, ketones, cholesterol, triglycerides, alcohols, amino acids, neurotransmitters, hormones, disease biomarkers (e.g., cancer biomarkers, cardiovascular disease biomarkers), drugs, pH, cell count, vitals (e.g., heart rate, body temperature (such as skin temperature), blood pressure (such as systolic and/or diastolic blood pressure), respiratory rate), cardiovascular data (e.g., pacemaker data, arrhythmia data), body function data, meal or nutrition data (e.g., number of meals; timing of meals; number of calories; amount of carbohydrates, fats, sugars, etc.), physical activity or exercise data (e.g., time and/or duration of activity; activity type such as walking, running, swimming; strenuousness of the activity such as low, moderate, high; etc.), sleep data (e.g., number of hours of sleep, average hours of sleep, variability of hours of sleep, sleep-wake cycle data, data related to sleep apnea events, sleep fragmentation (such as fraction of nighttime hours awake between sleep episodes, etc.)), stress level data (e.g., cortisol and/or other chemical indicators of stress levels, perspiration), a1c data, user location (e.g., GPS coordinates, elevation data), air pressure, humidity, temperature, air quality, and/or the like.

For example, the biosensor can be or include a blood glucose sensor. The blood glucose sensor can be any device capable of obtaining blood glucose data from a user. In some embodiments, the blood glucose sensor is configured to obtain samples from the user and determine glucose levels in the sample. Any suitable technique for obtaining user samples and/or determining glucose levels in the samples can be used. In some embodiments, for example, the blood glucose sensor is configured to detect substances (e.g., a substance indicative of glucose levels), measure a concentration of glucose, and/or measure another substance indicative of the concentration of glucose. The blood glucose sensor can be configured to analyze, for example, body fluids (e.g., blood, interstitial fluid, sweat, etc.), tissue (e.g., optical characteristics of body structures, anatomical features, skin, or body fluids), and/or vitals (e.g., heat rate, blood pressure, etc.) to periodically or continuously obtain blood glucose data. Optionally, the blood glucose sensor can include other capabilities, such as processing, transmitting, receiving, and/or other computing capabilities. In some embodiments, the blood glucose sensor can include at least one continuous glucose monitoring (CGM) device or sensor that measures the user's blood glucose level at predetermined time intervals. For example, the CGM device can obtain at least one blood glucose measurement every minute, 2 minutes, 5 minutes, 10 minutes, 15 minutes, 20 minutes, 30 minutes, 60 minutes, 2 hours, etc. In some embodiments, the time interval is within a range from 5 minutes to 10 minutes.

The biosensors described herein can also include various functionalities to facilitate data collection and/or processing. In some embodiments, for example, the biosensors are configured to perform one or more of the following functions: compensate for biofouling associated with body fluid-based monitoring, deliver medication, reduce or limit signal noise, compensate for time delays (e.g., with glucose changes for signal detection associated with body fluid-based detection), and/or manage over the air updates (e.g., algorithm updates, detection updates, software module updates).

### A. Multi-Analyte Biosensors

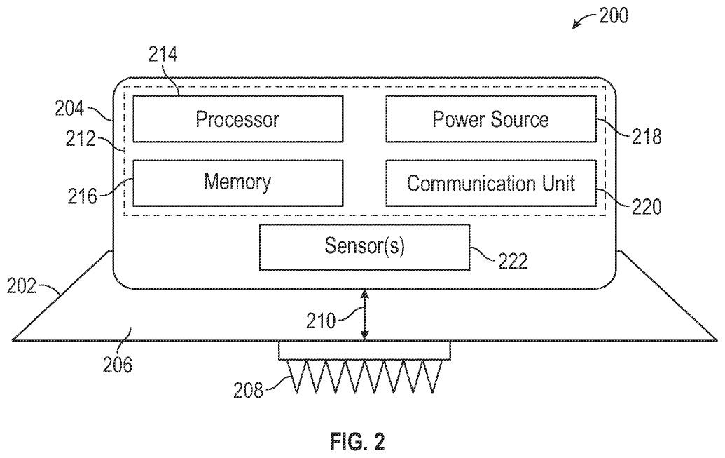

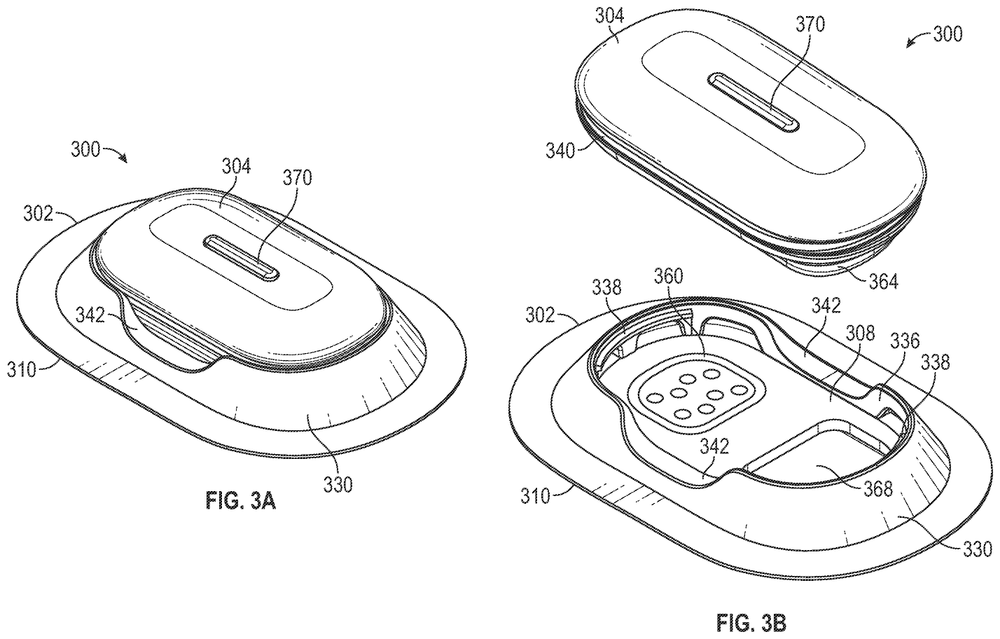

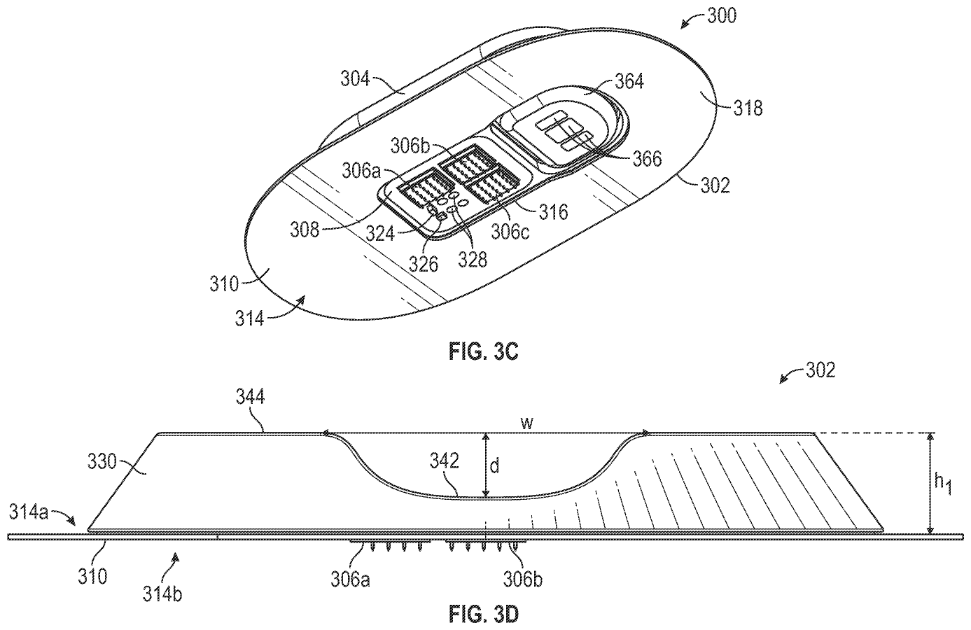

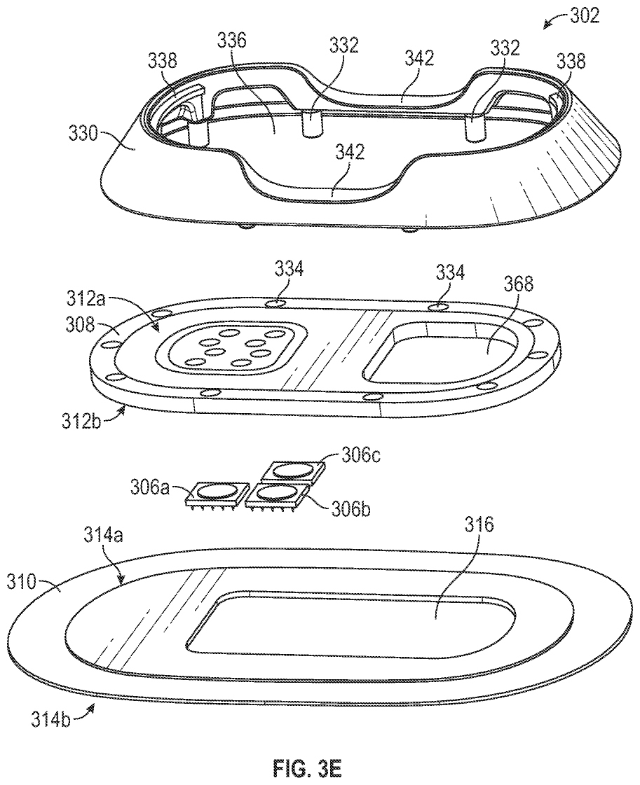

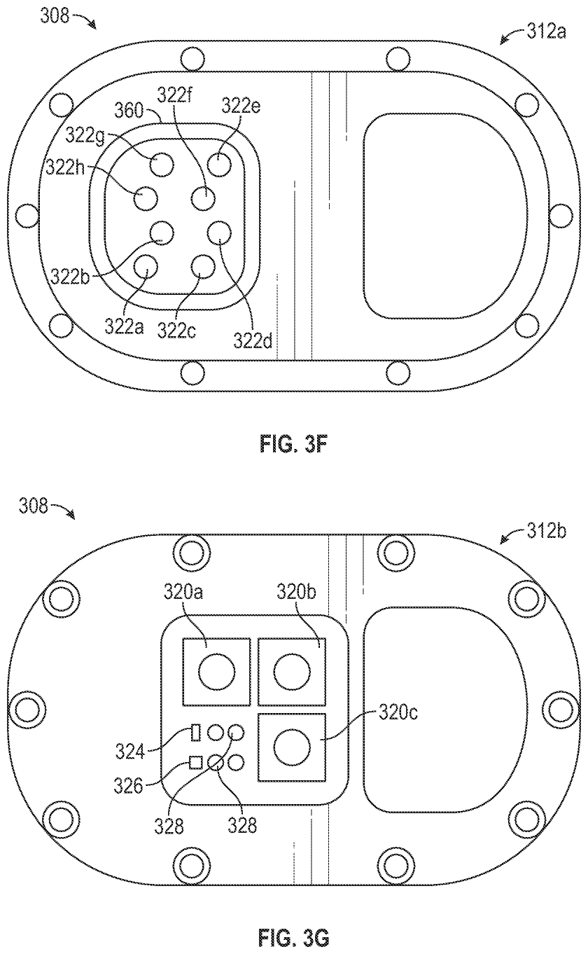

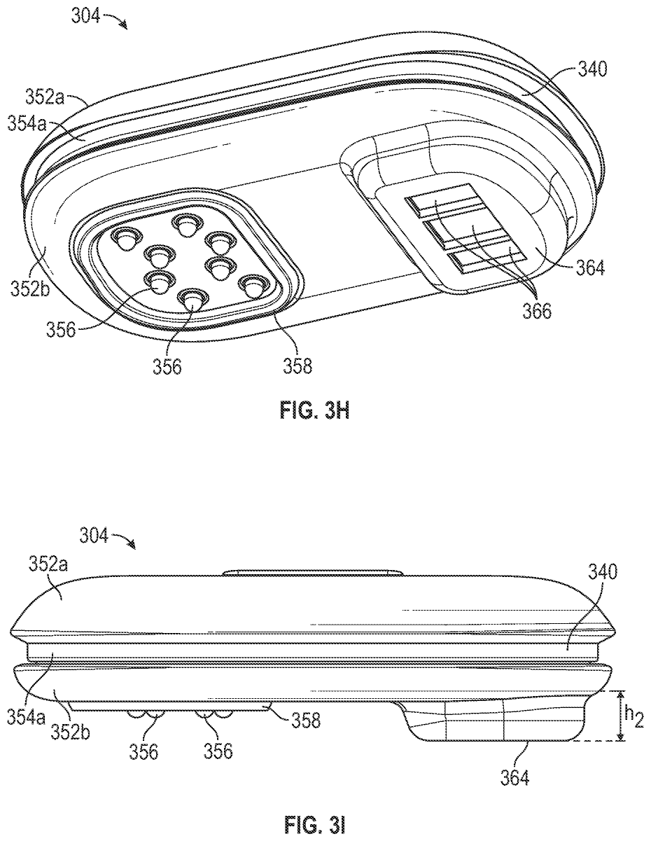

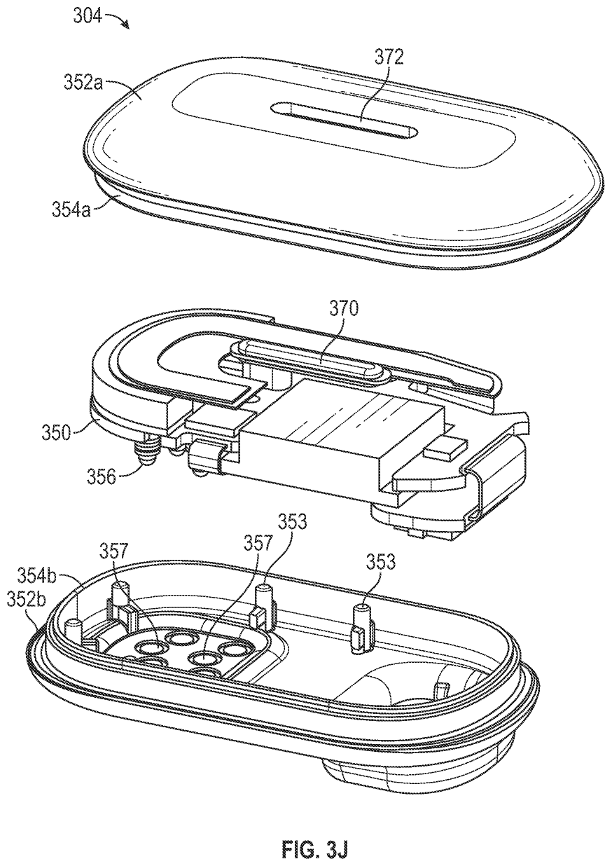

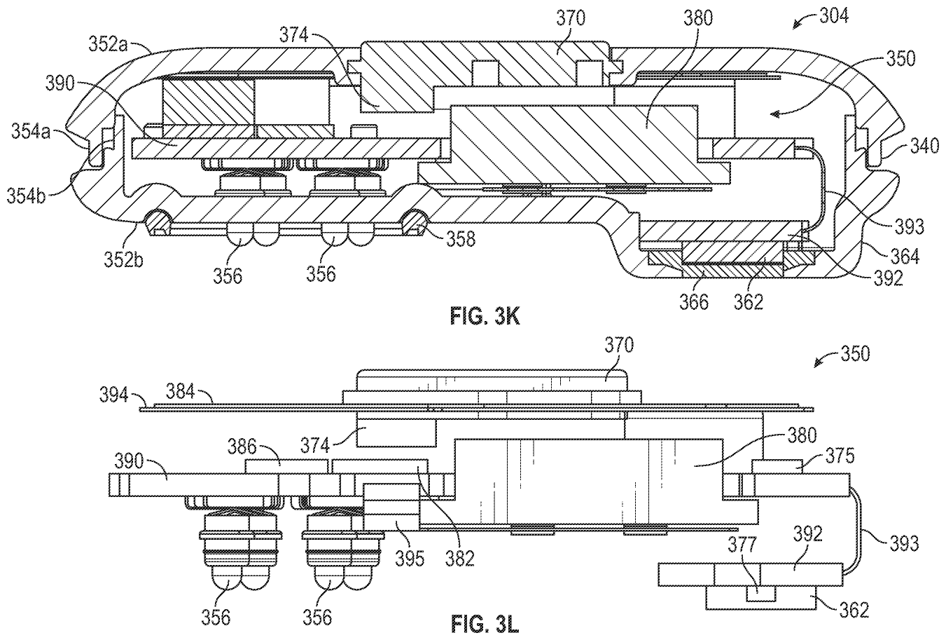

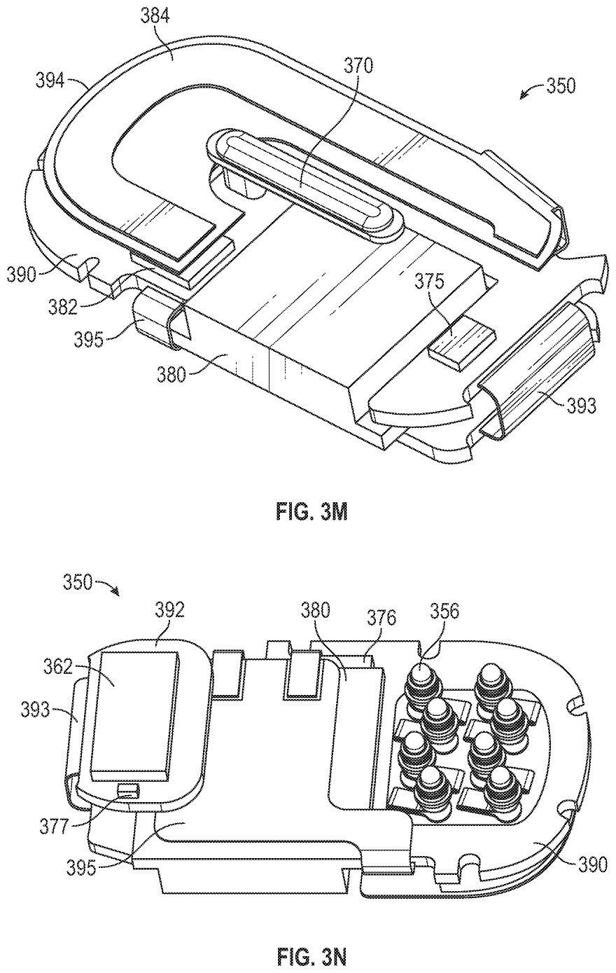

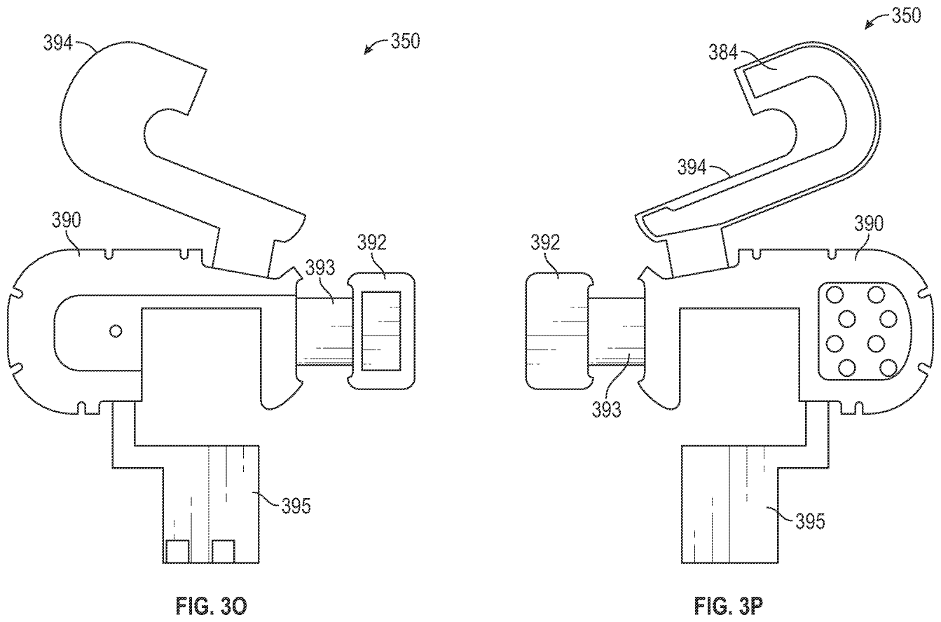

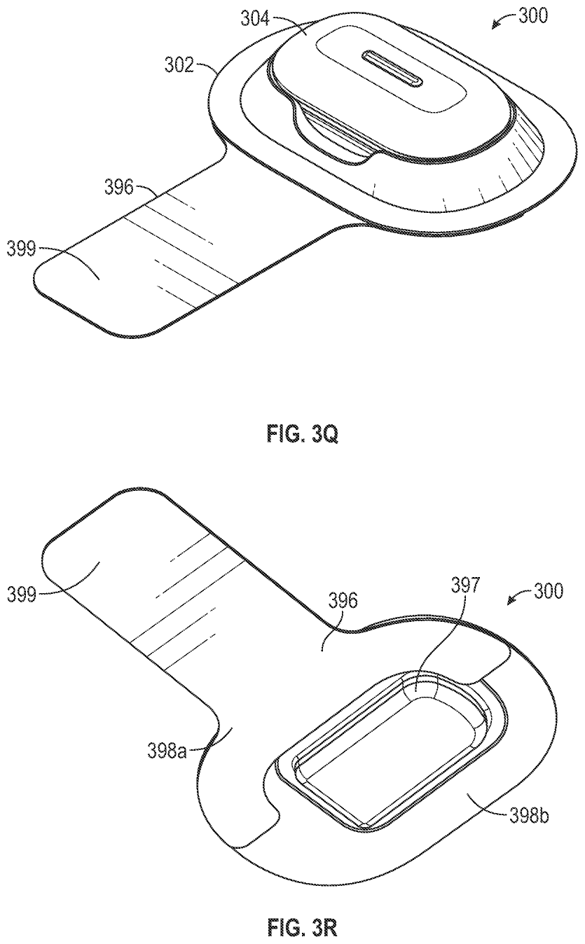

FIGS. 2-3R and the accompanying description provide various examples of biosensors that are suitable for use with the biomonitoring and healthcare guidance system 102 of FIG. 1. Specifically, FIG. 2 provides a general overview of the components of a biosensor, and FIGS. 3A-3R provide a representative example of a biosensor. Any of the features of the embodiments of FIGS. 2-3R can be combined with each other and/or with any of the other systems and devices described herein.

FIG. 2 is a schematic illustration of a biosensor device 200 (“device 200”) configured in accordance with embodiments of the present technology. The device 200 can be a wearable patch sensor configured to be applied to a user's body in order to obtain user health data in a non-invasive or minimally-invasive manner. The device 200 can be used in any of the systems and methods described herein (e.g., as the biosensor 104a of FIG. 1). The device 200 includes a patch 202 (also referred to as a “patch portion,” “base portion,” or “sensing component”) and a pod 204 (also referred to as a “pod portion,” “capsule portion,” or “electronics component”). The patch 202 can be coupled to the pod 204 (e.g., releasably coupled or permanently affixed) to form the device 200.

The patch 202 can include a substrate 206 configured to couple to the user's body (e.g., to the surface of the skin) via adhesives or other suitable temporary attachment techniques. The base portion also includes at least one array of microneedles 208 coupled to and/or supported by the substrate 206. The microneedles 208 can be configured to penetrate into the user's skin to access interstitial fluid therein. In some embodiments, when the device 200 is applied to the skin, the microneedles 208 extend only into the stratum corneum and epidermis, and do not penetrate into the dermis or hypodermis (subcutaneous tissue). This approach can reduce or avoid pain and/or discomfort, while still providing accurate detection of analytes in the epidermal interstitial fluid. The microneedles 208 can be configured to detect one or more analytes in the interstitial fluid, such as glucose, gases, electrolytes, BUN, creatinine, ketones, alcohols, amino acids, neurotransmitters, hormones, biomarkers, drugs, pH, cell count, and/or any of the other analytes described herein. Each microneedle 208 can be configured to detect a single analyte, or some or all of the microneedles 208 can be configured to detect multiple analytes (e.g., two, three, four, five, or more different analytes). Optionally, some or all of the microneedles 208 can be configured to detect physiological parameters, such as electrical properties (e.g., biopotential, bioimpedance), body temperature, etc.

The array can include any suitable number of microneedles 208 (e.g., 25 microneedles), and the microneedles 208 can be arranged in any suitable geometry (e.g., a 5×5 grid). Although FIG. 2 illustrates a single array of microneedles 208, in other embodiments, the device 200 can include two, three, four, five, or more arrays of microneedles 208. In embodiments where the device 200 includes multiple arrays, each array can be configured to perform a different function, or some of the arrays can perform the same function. For example, the device 200 can include a first array configured to detect a first set of analytes, a second array configured to detect a second set of analytes, a third array configured to detect a third set of analytes, and so on. Alternatively or in combination, the device 200 can include a first array configured as a working electrode, a second array configured as a reference electrode, and a third array configured as a counter electrode. Additional details and examples of microneedles and microneedle arrays suitable for use in the device 200 are described below in Section III.

The array of microneedles 208 can generate signals (e.g., electrical signals) indicative of health parameter values (e.g., analyte concentration and/or physiological values). For example, the array of microneedles 208 can generate a first electrical signal indicative of a first analyte, a second electrical signal indicative of a second analyte, and so on. Optionally, the array of microneedles 208 can generate at least a first electrical signal indicative of an analyte and at least a second electrical signal indicative of a physiological parameter. The array of microneedles 208 can be electrically coupled to the patch 202, which in turn can be electrically coupled to the pod 204 (schematically represented by arrow 210). The electrical connections between the array of microneedles 208, patch 202, and pod 204 can include any suitable combination of pins, contacts, wires, traces, etc. Accordingly, the signals generated by the microneedles 208 can be transmitted to the pod 204 for storage and/or processing.

The pod 204 can be a capsule, module, or other durable structure that couples to the patch 202 in order to assemble the device 200. The pod 204 can be mechanically coupled to the patch 202 using any suitable temporary or permanent attachment method, such as interference fit, snap fit, threading, fasteners, bonding, adhesives, and/or suitable combinations thereof. The pod 204 can include a casing or housing that encloses an electronics assembly 212 (also referred to herein as an “electronics subsystem”) of the device 200. The electronics assembly 212 can include one or more electronic components configured to perform the various operations described herein, such as a processor 214, memory 216, power source 218, and communication unit 220. Optionally, the pod 204 can also include one or more sensors 222 for measuring physiological parameters. The pod 204 can also include other electronic components not shown in FIG. 2, such as additional signal processing circuitry (e.g., multiplexer, analog front end (AFE), amplifier, filter, analog-to-digital converters (ADCs)), clock circuitry, power management circuitry, user input/output devices, and the like.

The processor 214 can be any component suitable for controlling the operations of the device 200, such as a microprocessor, microcontroller, field-programmable gate array (FPGA), application-specific integrated circuit (ASIC), and the like. For example, the processor 214 can receive and process signals generated by the array of microneedles 208 and/or the sensor(s) 222 in order to generate one or more measurements of health parameters (e.g., analyte levels, biopotential values, bioimpedance values, body temperature values, heart rate values, oxygen levels, etc.). In some embodiments, the processor 214 receives and processes at least a first electrical signal from the array of microneedles 208 to generate a first health measurement (e.g., an analyte level), and at least a second electrical signal from the sensor(s) 222 to generate a second health measurement (e.g., a physiological parameter). The processor 214 can be configured to receive and process any number of electrical signals (e.g., two, three, four, five, or more electrical signals) obtained by different sensing components of the device 200 to generate measurements of multiple health parameters (e.g., two, three, four, five or more different health parameters). Optionally, the processor 214 can use the health measurements to generate predictions, recommendations, notifications, etc. As another example, the processor 214 can control transmission of raw sensor data, processed data, health measurements, predictions, etc., to a remote device (e.g., a smartphone, smartwatch, or other user device or remote server). In a further example, the processor 214 can receive instructions from a remote device for controlling the operation of the device 200 (e.g., powering on, powering off, updating calibration and/or other signal processing parameters, device pairing, etc.). The processor 214 can also control the operations of the other components of the device 200 (e.g., operations of the memory 216, power source 218, communication unit 220, other sensor(s) 222, etc.).

The memory 216 can store instructions to be executed by the processor 214 and/or data generated during operation of the device 200. For example, the memory 216 can store raw and/or processed sensor data, as well as generated health measurements, predictions, recommendations, notifications, etc. The memory 216 can also store operating parameters for the device 200, such as calibration parameters, signal processing parameters, algorithms or programs (e.g., for generating health measurements, predictions, etc.), and so on. The memory 216 can include any suitable combination of volatile and non-volatile memory, such as flash memory, EEPROM, etc.

The power source 218 can be any component suitable for powering the operations of the device 200, such as a rechargeable battery, non-rechargeable battery, or suitable combinations thereof. The power source 218 can output power to the array of microneedles 208, processor 214, memory 216, communication unit 220, sensor(s) 222, and/or any other electronic components on the patch 202 or pod 204. The power source 218 can include or be operably coupled to power management circuitry (not shown). The power management circuitry can detect the charge status of the power source 218 (e.g., fully charged, partially charged, low charge), can allow the device 200 to operate in various modes (e.g., low power, full power), and/or any other suitable power-related function.

The communication unit 220 can allow the device 200 to transmit data to and/or receive data from a remote device (e.g., a mobile device, smartwatch, remote server, etc.). The communication unit 220 can be configured to communicate via any suitable combination of wired and/or wireless communication modes. In some embodiments, for example, the communication unit 220 uses Bluetooth Low Energy (BLE) to transmit and receive data.

The sensor(s) 222 can include any suitable combination of sensors for monitoring various health parameters, such as an optical sensor (e.g., photoplethysmography (PPG) sensor, pulse oximeter), heart rate sensor, blood pressure sensor, electrocardiogram (ECG) sensor, activity or motion sensor (e.g., accelerometer, gyroscope), temperature sensor (e.g., thermistor), location sensor, humidity sensor, etc. Each sensor can generate a respective set of signals, which can be received and processed by the processor 214 to generate health measurements and/or other user data. In some embodiments, the device 200 includes at least one, two, three, four, five, or more different sensors 222 for measuring physiological and/or other user parameters. Each sensor 222 can be located at any suitable region of the pod 204, such as at or near an upper surface, lower surface, lateral surface, or within an interior cavity of the pod 204. In other embodiments, however, some or all of the sensor(s) 222 can instead be located in the patch 202, rather than in the pod 204. For example, a temperature sensor can be located in the patch 202 in order to generate measurements of the user's skin temperature.

In some embodiments, the patch 202 is a disposable component that is configured for short-term use (e.g., no more than 4 weeks, 3 weeks, 2 weeks, 1 week, 6 days, 5 days, 4 days, 3 days, 2 days, 1 day, 12 hours, etc.), while the pod 204 is a reusable component that is configured for longer-term use (e.g., at least 1 week, 2 weeks, 3 weeks, 4 weeks, 1 month, 2 months, 3 months, 6 months, 1 year, etc.). This approach can be advantageous for reducing overall cost of the device 200, particularly in embodiments where the pod 204 includes more expensive components (e.g., the electronics assembly 212 and/or other sensor(s) 222). In such embodiments, the reusable pod 204 can be coupled to the disposable patch 202 to assemble the device 200 for use, and can be decoupled from the disposable patch 202 when the disposable patch 202 is to be replaced. As such, a single reusable pod 204 can be used with multiple different disposable patches 202, which can reduce the overall cost of the device 200, and enhance device longevity and adaptability. Optionally, a single reusable pod 204 can be used with multiple disposable patches 202 that detect different types of analytes. For example, the reusable pod 204 can be configured to interface with a first disposable patch 202 configured to detect a first set of analytes, a second disposable patch 202 configured to detect a second set of analytes, a third disposable patch 202 configured to detect a third set of analytes, and so on. In other embodiments, however, the patch 202 and pod 204 can both be disposable components, or can both be reusable components.

The device 200 can be configured to obtain and process the signals generated by the array of microneedles 208 and/or the sensor(s) 222 in order to determine measurements for one or more health parameters, such as measurements of glucose, gases, electrolytes, BUN, creatinine, ketones, cholesterol, alcohols, amino acids, neurotransmitters, hormones, disease biomarkers, drugs, pH, cell count, heart rate, body temperature, blood pressure, respiratory rate, cardiovascular data, body function data, meal or nutrition data, physical activity or exercise data, sleep data, stress level data, a1c data, and so on. In some embodiments, the electronics assembly 212 is configured to implement one or more algorithms, such as algorithms for sensor calibration, signal conditioning, determining presence of and/or values for health parameters based on the sensor signals, predicting current and/or future values for health parameters based on the sensor signals, etc. The algorithms can be stored locally at the electronics assembly 212 (e.g., in the memory 216) such that the device 200 can operate without being in communication with a separate computing device or system (e.g., a cloud computing network, remote server, user device, etc.). In such embodiments, the locally-stored algorithms can be periodically updated, e.g., via firmware updates and/or other modifications received from the separate computing device by the communication unit 220. Alternatively or in combination, some or all of the algorithms can be stored at the separate computing device or system. In some embodiments, local processing can be performed onboard the device 200 for certain situations (e.g., when network connectivity is lost), while processing can be performed at a separate computing device or system in other situations (e.g., when network connectivity is available).

The operation of the device 200 can be customized based on the particular health parameters to be detected. For example, the patch 202 can include a respective memory (not shown) configured to store identifier information for the patch 202, such as the type and/or configuration of the microneedles 208, the type and/or configuration of the microneedle arrays, the types of analytes and/or physiological parameters detected by the microneedles 208, the types of other sensors included in the patch 202, a unique patch ID (e.g., a serial number), a lot ID, manufacturing date, expiration date and/or expected lifetime, and/or any other suitable information. In some embodiments, the processor 214 is configured to detect when the pod 204 is coupled to the patch 202. Once the pod 204 is connected to the patch 202, the processor 214 can interrogate or otherwise communicate with the patch 202 to detect the identifier information for the patch 202. The processor 214 can access and read the identifier information, and can then adjust the parameters and/or algorithms used to process the electrical signals generated by the patch 202 (e.g., by the microneedles 208), based on the identifier information. For example, the processor 214 can use the identifier information to determine detection capabilities of the patch 202 (e.g., which analytes and/or physiological values the patch 202 is configured to detect). The processor 214 can select an appropriate locally-stored algorithm for processing the signals generated by the patch 202 and/or determining health parameters from the signals. The algorithm can vary depending on the microneedle type and/or configuration, type of detected analyte or parameter, the manufacturing information for the patch 202 (e.g., batch or lot ID), the expected lifetime of the patch 202, other available sensor data, or any other suitable factor. Additionally, parameter detection can be performed using different algorithms used with different groups of users and algorithms selected based on user health data. The locally-stored algorithms can be updated based on the health parameters (e.g., via updates received from a separate user device, cloud computing system, etc.).

In embodiments where the pod 204 is configured for use with multiple patches 202 having different functionalities (e.g., different detection capabilities), when the pod 204 is coupled to a new patch 202, the processor 214 can use the identifier information received from the patch 202 to assess the functionality of the patch 202. If the processor 214 determines that the patch 202 has newly available functionality that the processor 214 is not currently programmed to accommodate, the processor 214 can retrieve the appropriate algorithms, calibration parameters, signal processing parameters, and/or other updates from a remote device (e.g., a user device, cloud computing system, etc.). Accordingly, the software implemented by the pod 204 can be rapidly and dynamically updated to accommodate different and/or new patch functionalities.

The health measurements produced by the device 200 can be used to generate personalized healthcare guidance, such as one or more predictions, recommendations, suggestions, feedback, and/or diagnosis for a number of diseases, conditions, or health states. For example, blood pressure can be monitored and/or predicted based on optical data (e.g., PPG data), electrical data (e.g., ECG data), heart rate data, user data, and/or activity data. As another example, sleep (e.g., sleep patterns, sleep quality) can be tracked and/or predicted based on heart rate data, skin temperature data, and/or activity data. In a further example, respiratory illness (e.g., COVID-19, allergies, infections etc.) can be monitored and/or predicted based on skin temperature data, blood pressure data, and/or respiration rate. The health measurements can be used to detect a condition, distinguish between different conditions (e.g., infection versus allergies), and/or monitor the progression of the condition. In yet another example, fertility can be tracked and/or predicted based on skin temperature data. The personalized guidance can be generated based solely on the health measurements from the device 200, or can be generated through a combination of health measurements and other information (e.g., information from any number of sensor data streams, user data sets, etc.). The healthcare guidance can be generated locally onboard the device 200, by a user device that receives health measurement data from the device 200 (e.g., via a mobile application on a user's smartphone or smartwatch), by a cloud computing system or remote server that receives health measurement data from the device 200, or any suitable combination thereof.

The configuration of the device 200 shown in FIG. 2 can be modified in many different ways. For example, in other embodiments, the array of microneedles 208 can be omitted such that the device 200 does not include or otherwise use microneedle-based analyte detection. In such embodiments, the patch 202 may include other sensor types (e.g., a temperature sensor, a metal electrode for ECG sensing), or may not include any sensors at all, such that all sensing operations are performed by the sensor(s) 222 (e.g., motion sensor, optical sensor, etc.) located in the pod 204. As another example, any of the components of the device 200 can be separated into discrete subcomponents (e.g., multiple processors 214, multiple memories 216, etc.), combined into a single component (e.g., the processor 214 and communication unit 220 can be integrated into a single chip), or omitted altogether. In a further example, any of the components of the device 200 can be positioned at different locations (e.g., some or all of the sensor(s) 222 can be located on the patch 202 instead of the pod 204).

FIGS. 3A-3R illustrate a representative example of a biosensor device 300 (“device 300”) configured in accordance with embodiments of the present technology. Specifically, FIGS. 3A-3C illustrate the overall device 300, FIGS. 3D-3G illustrate a patch portion 302 (“patch 302”) of the device 300, FIGS. 3H-3P illustrate a pod portion 304 (“pod 304”) of the device 300, and FIGS. 3Q and 3R illustrate the device 300 with packaging components.

Referring first to FIG. 3A (top perspective view), 3B (exploded view), and 3C (bottom perspective view) together, the device 300 is configured as a wearable sensor for application to the user's body. The device 300 includes a patch 302 for mounting to the skin, and a pod 304 that interfaces with the patch 302. The patch 302 and pod 304 can be discrete components that are releasably connected to each other to form the device 300 (FIGS. 3A and 3C show the device 300 when assembled, and FIG. 3B shows the device 300 when the patch 302 and the pod 304 are separated). As previously discussed, the patch 302 can be a disposable component intended for short-term use, while the pod 304 can be a reusable component intended for longer-term use with multiple different patches 302.

The device 300 can be configured to be worn by the user over an extended period of time in order to generate measurements of any of the health parameters described herein, such as analyte levels (e.g., concentrations of glucose, gases, electrolytes, BUN, creatinine, ketones, cholesterol, triglycerides, alcohols, amino acids, neurotransmitters, hormones, disease biomarkers, drugs, etc.), physiological information (e.g., heart rate, body temperature, blood oxygenation, blood pressure, respiratory rate, bioimpedance, activity levels, sleep data), etc. In some embodiments, the device 300 includes a plurality of different sensor types for measuring multiple health parameters. For example, the device 300 can include at least two, three, four, five, or more different sensor types. The sensors can be located in the patch 302, pod 304, or any suitable combination thereof.

FIG. 3D is a side view of the patch 302, and FIG. 3E is an exploded view of the patch 302. Referring next to FIGS. 3B-3E together, the patch 302 is configured to temporarily attach to the user's body, such as on the skin of the user's hand, arm, shoulder, leg, foot, chest, back, neck, etc. The patch 302 can include one or more sensors that generate signals indicative of analyte levels, physiological parameters, and/or other health parameters associated with the user's skin. As best seen in FIG. 3C, the patch 302 can include a set of microneedle arrays 306a-c configured to penetrate into the user's skin (e.g., into the epidermis). The microneedle arrays 306a-c can incorporate any of the features described above with respect to the microneedles 208 of FIG. 2 and/or discussed further below in Section III.

In the illustrated embodiment, the patch 302 includes three microneedle arrays 306a-c, each including 25 microneedles arranged in a 5×5 grid. The microneedle arrays 306a-c can be configured to detect one or more analytes in the interstitial fluid of the epidermis, e.g., using electrochemical techniques. For example, the microneedle array 306a can be configured as a first working electrode for detecting a first set of analytes (e.g., glucose), the microneedle array 306b can be configured as a reference electrode, and the microneedle array 306c can be configured as a counter electrode. In other embodiments, however, the patch 302 can include fewer or more microneedle arrays 306a-c, and/or the configuration of each array 306a-c (e.g., geometry, number of microneedles, detected analyte, etc.) can be varied as desired. For example, the patch 302 can include four microneedle arrays, with two arrays configured as working electrodes, one array configured as a reference electrode, and one array configured as a counter electrode.

Optionally, some or all of the microneedle arrays 306a-c can alternatively or additionally detect other parameters besides analyte concentration, such as bioimpedance, biopotential, etc. For example, bioimpedance can be used to assess various physiological parameters, such as respiration rate, body composition, and/or hydration. Additionally, bioimpedance measurements of individual microneedles and/or microneedle arrays 306a-c can be used to measure or estimate microneedle penetration into the skin (e.g., whether the arrays 306a-c are in proper contact with the skin, the percentage of microneedles in each array that are in proper contact with the skin, etc.). The amount of microneedle penetration can be used to adjust downstream signal processing performed by the device 300, such as selecting correction factors for signal processing algorithms, selecting the algorithms to be used, selecting subsets of data to be used or excluded, etc.

As best seen in FIG. 3E, the patch 302 can include an electronics substrate 308 (e.g., a printed circuit board (PCB), a flex circuit, etc.) and a mounting substrate 310 (e.g., an adhesive film, sticker, tape, etc.) that collectively support the microneedle arrays 306a-c and couple to the user's body. The electronics substrate 308 can be a flattened, oval-shaped structure having an upper surface 312a and a lower surface 312b, and the mounting substrate 310 can also be a flattened, oval-shaped structure having an upper surface 314a and a lower surface 314b. In other embodiments, the electronics substrate 308 and mounting substrate 310 can each independently have a different shape (e.g., circular, square, rectangular, etc.). Additionally, although the mounting substrate 310 is illustrated as being larger than the electronics substrate 308 (e.g., with respect to length, width, perimeter, etc.), in other embodiments, the mounting substrate 310 can be the same size as the electronics substrate 308 or can be smaller than the electronics substrate 308. Moreover, in other embodiments, the electronics substrate 308 and mounting substrate 310 can be combined into a single, unitary component, rather than being two discrete components that are connected to each other to assemble the patch 302.

The microneedle arrays 306a-c can be coupled to the lower surface 312b of the electronics substrate 308. The mounting substrate 310 can include an aperture 316 configured such that, when the lower surface 312b of the electronics substrate 308 is attached to the upper surface 314a of the mounting substrate 310, the microneedle arrays 306a-c pass through the aperture 316 and extend past the lower surface 314b of the mounting substrate 310 in order to access the user's skin (best seen in FIGS. 3C and 3D). Optionally, the aperture 316 of the mounting substrate 310 can be larger than the surface area of the microneedle arrays 306a-c so that one or more additional sensors can extend through the mounting substrate 310 to access the skin, as described further below.

Referring to FIG. 3C, the mounting substrate 310 can be configured to temporarily secure the patch 302 (as well as the rest of the device 300) to the user's skin. For example, the lower surface 314b of the mounting substrate 310 can include an adhesive region 318 configured to temporarily attach to the user's skin. The adhesive region 318 can extend across the entirety of the lower surface 314b, or at least portions thereof. In the illustrated embodiment, the microneedle arrays 306a-c are located at the central portion of the mounting substrate 310, such that the adhesive region 318 completely surrounds the microneedle arrays 306a-c to maintain the microneedle arrays 306a-c in close contact with the skin. In other embodiments, however, the microneedle arrays 306a-c and/or adhesive region 318 can be arranged differently, e.g., the microneedle arrays 306a-c can be offset to one side of the mounting substrate 310, the adhesive region 318 can surround only a portion of the microneedle arrays 306a-c, etc. The adhesive region 318 can be made of any suitable material suitable for coupling to the skin for an extended time period (e.g., at least 12 hours, 24 hours, 2 days, 3 days, 4 days, 5 days, 6 days, 1 week, etc.). The material of the adhesive region 318 can also be biocompatible, breathable, and/or water-resistant, e.g., to reduce discomfort and/or avoid premature detachment. Additionally, the mounting substrate 310 itself can be a flexible component configured to conform the user's body to further improve adhesion and user comfort.

FIGS. 3F and 3G illustrate top and bottom views of the electronics substrate 308, respectively. The electronics substrate 308 can be configured to electrically and mechanically couple the microneedle arrays 306a-c to the other portions of the device 300. In the illustrated embodiment, the lower surface 312b of the electronics substrate 308 includes a set of lower electrical contacts (e.g., conductive regions 320a-c shown in FIG. 3G), and the upper surface 312a of the electronics substrate 308 includes a corresponding set of upper electrical contacts (e.g., pin contacts 322a-h shown in FIG. 3F). The lower electrical contacts can be electrically coupled to the upper electrical contacts via wires, traces, or other conductive elements extending through the thickness of the electronics substrate 308. For example, each conductive region 320a-c can be electrically coupled to a corresponding pair of pin contacts 322a-h (e.g., conductive region 320a is connected to pin contacts 322a-b, conductive region 320b is connected to pin contacts 322c-d, conductive region 320c is connected to pin contacts 322e-f, etc.). Accordingly, the microneedle arrays 306a-c can transmit sensor signals to the lower electrical contacts, which in turn can transmit the signals to upper electrical contacts, which in turn can transmit the signals to another portion of the device 300 (e.g., to the pod 304).

In some embodiments, the patch 302 includes additional functional components. For example, as shown in FIGS. 3C and 3G, the patch 302 can include at least one temperature sensor 324 (e.g., a thermistor) for measuring the user's body temperature (e.g., skin temperature). The temperature sensor 324 can be located on the lower surface 312b of the electronics substrate 308 (e.g., near the microneedle arrays 306a-c) and can be exposed by the aperture 316 of the mounting substrate 310. Accordingly, when the patch 302 is coupled to the user's body, the temperature sensor 324 can be near or in direct contact with the skin for high accuracy skin temperature measurements. The temperature sensor 324 can be electrically coupled to the pin contacts 322g-h to allow for communication with other portions of the device 300 (e.g., the pod 304). The signals generated by the temperature sensor 324 can be used to measure body temperature and/or adjust signal processing parameters (e.g., in embodiments where the operations of the microneedle arrays 306a-c and/or other sensors are at least partially dependent on body temperature).

The patch 302 can also include a memory 326 (e.g., an EEPROM) for storing information related to the patch 302, such as identifier information. As described above, the identifier information can include the types of the microneedle arrays 306a-c, the types of analytes detected by the microneedle arrays 306a-c, the configuration of the microneedle arrays 306a-c, the types of other sensors included in the patch 302, a unique patch ID (e.g., a serial number), a lot ID, manufacturing date, expiration date, and/or any other suitable information. The memory 326 can be electrically coupled to the pin contacts 322g-h to allow for communication with other portions of the device 300 (e.g., the pod 304). Optionally, the patch 302 can include one or more test points or contacts 328 that are electrically coupled to the memory 326 to allow for programming of and/or reading from the memory 326. For example, the test points 328 can be used to input the identifier information into the memory 326, e.g., during the manufacturing process. Although the memory 326 and test points 328 are depicted as being on the lower surface 312b of the electronics substrate 308, in other embodiments, the memory 326 and/or test points 328 can be located at other portions of the patch 302.

Referring to FIGS. 3A, 3B, 3D, and 3E together, the patch 302 can include a housing 330 configured to couple to the pod 304. In the illustrated embodiment, the housing 330 is mounted on the upper surfaces 312a, 314a of the electronics substrate 308 and mounting substrate 310, respectively. For example, as best seen in FIG. 3E, the housing 330 can include a plurality of stakes or posts 332 configured to fit into corresponding holes 334 at the periphery in the electronic substrate 308 so that the housing 330, electronics substrate 308, and mounting substrate 310 can be permanently affixed to each other by heat staking. Alternatively or in combination, other techniques for connecting the housing 330, electronics substrate 308, and mounting substrate 310 to each other can be used. As shown in FIG. 3D, when assembled, the housing 330 can extend above the upper surface 314a of the mounting substrate 310, e.g., by a height h₁ greater than or equal to 1 mm, 1.5 mm, 2 mm, 2.5 mm, 3 mm, 3.5 mm, 4 mm, 4.5 mm, or 5 mm. Although the housing 330 is depicted as having inwardly-sloped sidewalls, in other embodiments, the sidewalls can be substantially vertical, can slope outwards, or can have any other suitable geometry.

The housing 330 can be a continuous, annular structure having an aperture 336 shaped to receive at least a portion of the pod 304. As best seen in FIG. 3B, the aperture 336 can have a size and shape similar to the size and shape of the pod 304 (e.g., an oval shape or other suitable shape). For example, the aperture 336 can have a length within a range from 20 mm to 40 mm (e.g., approximately 30 mm), and/or a width within a range from 15 mm to 35 mm (e.g., approximately 25 mm). The aperture 336 can be sized such that the upper electrical contacts of the electronics substrate 308 are exposed for coupling to corresponding contacts on the pod 304, as described further below. The housing 330 can connect to the pod 304 using any suitable temporary mechanism, such as snap fit, interference fit, threading, fasteners, etc. For example, the inner surface of the housing 330 can include a set of ridges 338 configured to mate with a corresponding groove 340 formed in the periphery of the pod 304 (FIG. 3B) via snap fit. In the illustrated embodiment, the housing 330 includes two ridges 338 located at opposite ends of the housing 330. In other embodiments, however, the number and/or locations of the ridges 338 can be varied as desired, e.g., depending on the configuration of the groove 340. The housing 330 can be made of a relatively rigid material (e.g., plastic) to further ensure secure coupling to the pod 304.

Optionally, the housing 330 can include a set of cutouts 342. The cutouts 342 can extend from an upper edge 344 of the housing 330 (FIG. 3D) towards the upper surface 314a of the mounting substrate 310. The geometry (e.g., size and/or shape) and locations of the cutouts 342 can be varied as desired. In the illustrated embodiment, for example, the housing 330 includes two U-shaped cutouts 342 located at opposite lateral sides of the housing 330. In other embodiments, the housing 330 can include a different number of cutouts 342, the cutouts 342 can have different shapes and/or be at different locations, etc. Each cutout 342 can have a depth d less than or equal to 5 mm, 4.5 mm, 4 mm, 3.5 mm, 3 mm, 2.5 mm, 2 mm, 1.5 mm, or 1 mm; and/or a width w less than or equal to 20 mm, 15 mm, 10 mm, 5 mm, or 1 mm. As best seen in FIG. 3A, the cutouts 342 can optionally expose the lateral surfaces of the pod 304 to allow a user to manually separate the pod 304 from the patch 302, e.g., by gripping the exposed surfaces of the pod 304 with the fingers, by inserting a removal tool into the cutout 342 and between the housing 330 and the pod 304, etc. In other embodiments, however, the cutouts 342 may be provided merely for aesthetic and/or other purposes, or can be omitted altogether.

Referring next to FIGS. 3H (bottom perspective view of the pod 304), 3I (side view), 3J (exploded view), and 3K (cross-sectional view) together, the pod 304 can be a reusable capsule or container that houses an electronics assembly 350 (FIG. 3J) of the device 300. In the illustrated embodiment, the pod 304 is an elongate structure having a generally oval, rounded shape. In other embodiments, however, the pod 304 can have a different shape (e.g., having a square, rectangular, circular, or any other suitable shape; having sharper corners and edges; etc.). The dimensions of the pod 304 can be varied as desired. For example, the pod 304 can have a length within a range from 20 mm to 40 mm (e.g., approximately 30 mm) and/or a width within a range from 10 mm to 30 mm (e.g., approximately 18 mm).

The pod 304 can include an upper pod housing 352a (“upper housing 352a”) and a lower pod housing 352b (“lower housing 352b”) that connect to each other to enclose and protect the electronics assembly 350. The upper and lower housings 352a-b can each be made of a durable, rigid, and/or watertight material (e.g., plastic), which can be advantageous for limiting fluid ingress into the pod 304, prolonging the usable lifespan of the pod 304, avoiding inadvertent damage during use, and/or facilitating cleaning. The upper and lower housings 352a-b can be manufactured as separate components and subsequently mechanically coupled to each other. For example, as best seen in FIG. 3J, the lower housing 352b can include a plurality of stakes or posts 353 so that the upper housing 352a, lower housing 352b, and electronics assembly 350 can be permanently affixed to each other by heat staking. In other embodiments, however, other techniques for connecting the upper housing 352a, lower housing 352b, and electronics assembly 350 can be used.

As best seen in FIG. 3J, the upper housing 352a can include an upper annular wall 354a that is recessed from the lateral edges of the upper housing 352a, and the lower housing 352b can include a lower annular wall 354b that is recessed from the lateral edges of the lower housing 352b. When the upper housing 352a is connected to the lower housing 352b (FIGS. 3H and 3I), the lower wall 354b can fit within the upper wall 354b, such that the upper wall 354b defines and forms the groove 340 around the pod 304. As discussed above, the groove 340 can receive and engage the ridges 338 on the housing 330 of the patch 302 (FIG. 3B) to couple the pod 304 to the patch 302 via snap fit. Although the illustrated embodiment shows the groove 340 extending around the entire periphery of the pod 304, in other embodiments, the groove 340 can be localized to discrete portions of the pod 304 (e.g., only at the ends of the pod 304).

The electronics assembly 350 can include a set of electrical contacts (e.g., pins 356) for coupling to the electrical contacts (e.g., pin contacts 322a-h—FIG. 3F) of the patch 302. For example, the pins 356 can be spring-loaded pins (pogo pins), each of which can be electrically connected to an individual corresponding pin contact to transmit signals between the electronics assembly 350 and the patch 302. The lower surface of the lower housing 352b can include a plurality of holes 357 (FIG. 3J) to receive and expose the pins 356. As best seen in FIG. 3H, when the pod 304 is assembled, the tips of the pins 356 can protrude outward from the lower housing 352b to access to the pin contacts 322a-h of the patch 302.

Optionally, the lower surface of the lower housing 552b can include a seal 358 (e.g., an O-ring, gasket, etc.) made of silicone, rubber, or other elastomeric material. When the pod 304 is coupled to the patch 302, the seal 358 can contact a corresponding region 360 (FIG. 3F) on the electronics substrate 308 of the patch 302 to create a watertight seal preventing moisture from reaching the pins 356 and pin contacts 322a-g. Although the illustrated embodiment shows a single seal 358 surrounding all of the pins 356, in other embodiments, the seal 358 can surround only some of the pins 356, or the pod 304 can include seals each surrounding a respective subset of pins 356, etc. Moreover, although the seal 358 is depicted as being localized to the portion of lower housing 552b near the pins 356, in other embodiments, the seal 358 can instead be located along the perimeter of the lower housing 552b to contact the inner walls of the housing 330 of the patch 302.

Referring next to FIGS. 3L (side view of the electronics assembly 350), 3M (top perspective view), and 3N (bottom perspective view) together, the electronics assembly 350 can include one or more sensors for detecting physiological parameters. For example, as best seen in FIGS. 3L and 3N, the electronics assembly 350 can include an optical sensor 362 for measuring heart rate, blood oxygenation, and/or other analytes and/or parameters using light. For example, the optical sensor 362 can be or include a PPG sensor or pulse oximeter. The optical sensor 362 can include one or more light sources (e.g., LEDs) configured to emit one or more wavelengths of light, light detectors (e.g., photodiodes) configured to detect one or more wavelengths of light, filters, etc. The number and types of light sources, light detectors, and filters can be configured in any suitable arrangement known to those of skill in the art. In some embodiments, for example, the optical sensor 362 includes four LEDs (e.g., two green LEDs, one red LED, one infrared (IR) LED) and two photodiodes (e.g., one broad spectrum photodiode and one IR-optimized photodiode).

Referring again to FIGS. 3H, 3I, and 3K together, the optical sensor 362 can be located within a protruding portion 364 of the lower housing 352b. The protruding portion 364 can be located at the end of the pod 304 opposite from the pins 356. The protruding portion 364 can have any suitable geometry. For example, the protruding portion 364 can have a height h₂ (FIG. 3I) of at least 0.5 mm, 0.75 mm, 1 mm, 1.25 mm, 1.5 mm, 1.75 mm, 2 mm, 2.25 mm, or 2.5 mm from the surface of the lower housing 352b. Although the protruding portion 364 is depicted as having a semi-oval shape, in other embodiments, the protruding portion 364 can have a different shape (e.g., semi-circular, oval, circular, square, rectangular, etc.). As best seen in FIGS. 3H and 3K, the protruding portion 364 can have a generally flattened lower surface including one or more windows 366 (e.g., three windows 366). The windows 366 can provide an optical path through the lower housing 352b so light can be transmitted from and/or received by the optical sensor 362. The windows 366 can be made of any suitable optically transmissive and/or transparent material, such as plastic or glass.

Referring next to FIGS. 3C and 3E together, the patch 302 can include an opening or passage that accommodates the protruding portion 364. As best seen in FIG. 3E, the electronics substrate 308 can include an aperture 368 sized and shaped similarly to the protruding portion 364. Additionally, as previously described, the mounting substrate 310 can include the aperture 316 for exposing both the protruding portion 364 and the microneedle arrays 306a-c, and the housing 330 can include the aperture 336 for receiving the pod 304. When the electronics substrate 308, mounting substrate 310, and housing 330 are assembled, the apertures 316, 336, and 368 can be aligned with each other to collectively form the opening for the protruding portion 364. As shown in FIG. 3C, when the pod 304 is coupled to the patch 302, the protruding portion 364 can fit within and/or pass through the opening in the patch 302, thus exposing the lower surface of the protruding portion 364 and the windows 366. Accordingly, when the patch 302 is applied to the user's body, the windows 366 can be placed near or in direct contact with the skin so the optical sensor 362 can transmit light to and/or receive light from the user's tissue via the windows 366.

Optionally, the protruding portion 364 can also be used to separate the pod 304 from the patch 302. For example, once the device 300 has been removed from the user's body, the user can press against the exposed lower surface of the protruding portion 364 to disengage the pod 304 from the patch 302. The pod 304 can be detached from the patch 302 when the pod 304 needs to be recharged, when the patch 302 is to be replaced, etc., as described further below.

Referring next to FIGS. 3K, 3L, and 3M together, the pod 304 can include at least one electrical contact 370 (e.g., an electrode, lead, etc.) for measuring bioelectrical properties (e.g., bioimpedance, biopotential, ECG, etc.). The number and locations of the electrical contact(s) 370 can be varied as appropriate to produce the desired measurements. In the illustrated embodiment, the electrical contact 370 is a conductive strip, contact, bar, etc., that fits within a slot 372 (FIG. 3J) in the upper housing 352a of the pod 304. Accordingly, when the pod 304 is assembled, the upper surface of the electrical contact 370 can be exposed for access by the user's finger, hand, or other body part. The electrical contact 370 can include at least one pin 374 extending into the interior of the pod 304 for connecting to the electronics assembly 350. Optionally, the electronics assembly 350 can include a spring contact or similar mechanism (omitted for purposes of simplicity) to provide direct contact between the pin 374 and the rest of the electronics assembly 350.

In some embodiments, the device 300 includes an ECG sensor in which the electrical contact 370 serves as a first ECG electrode, and at least one of the microneedle arrays 306a-c serves as a second ECG electrode. The first and second ECG electrodes can be used to generate cross-body ECG measurements. For example, the microneedle array serving as the second ECG electrode can be mounted on the user's arm, and the user can touch the electrical contact 370 with the fingers or hand of the opposite arm. The ECG measurements can be used to determine, for example, heart rate, cardiac arrhythmias, and/or other cardiovascular-related parameters. Optionally, the ECG measurements can be combined with PPG data from the optical sensor 362 to determine blood pressure levels, in accordance with techniques known to those of skill in the art.

Referring next to FIGS. 3L and 3N together, the device 300 can optionally include a motion or activity sensor 375. The motion sensor 375 can be or include one or more accelerometers, gyroscopes, or a combination thereof. The motion sensor 375 can generate signals indicative of user physical activity or exercise, sleep patterns, and/or any other motion-related parameters. Alternatively or in combination, the signals from the motion sensor 375 can be used to adjust the operation of the device 300, e.g., performing corrections if certain sensors are sensitive to motion, detecting conditions that are likely to lead to sensor dropout or other anomalies, etc.

Optionally, the pod 304 can include a temperature sensor 377 (e.g., a thermistor) for measuring the user's body temperature (e.g., skin temperature). When the pod 304 is assembled, the temperature sensor 377 can be positioned near the user's skin, e.g., within the protruding portion 364 of the lower housing 352b of the pod 304. In some embodiments, the temperature sensor 324 of the patch 302 serves as the primary temperature sensor for the device 300, and the temperature sensor 377 of the pod 304 serves as a secondary temperature sensor of the device 300 (e.g., for redundancy and/or accuracy purposes). In other embodiments, however, the temperature sensor 324 of the patch 302 can be omitted, such that the temperature sensor 377 of the pod 304 serves as the primary temperature sensor of the device 300.

Referring again to FIGS. 3L-3N together, the electronics assembly 350 can include additional components for performing various operations. For example, the electronics assembly 350 can include at least one processor 376 (FIG. 3N), at least one memory unit (obscured in the Figures), a rechargeable battery 380, a power management integrated circuit (PMIC) 382, an antenna 384, and a microneedle array interface 386 (“interface 386”—FIG. 3L). In other embodiments, however, any of these components can be combined with each other into a single component, separated into discrete subcomponents, replaced with other suitable components known to those of skill in the art, and/or omitted altogether. Additionally, the electronics assembly 350 can include other components not shown in the depicted embodiments (e.g., other types of sensors, circuitry, etc.).

The processor(s) 376 can be or include any number of microcontrollers, microprocessors, or other suitable components for performing and/or controlling various operations, such as any of the following: receiving and processing signals generated by the microneedle arrays 306a-c and/or other sensors of the device 300, determining measurements for health parameters based on the sensor signals, predicting current and/or future values for health parameters based on the sensor signals, performing sensor calibration routines, monitoring the operational status of the sensors, monitoring the charge status of the rechargeable battery 380, detecting identifier information for the patch 302, adjusting signal processing parameters (e.g., calibration parameters) and/or algorithms based on the detected identifier information, pairing with a remote device, transmitting data (e.g., raw or processed sensor signals, health measurements, predictions, notifications) to a remote device, receiving data (e.g., user data, calibration parameters, signal processing parameters, algorithms, firmware updates) from a remote device, and/or other suitable processes.

In some embodiments, the processor(s) 376 are dual-core processors that are part of a system-on-a-chip (SOC). For example, the SOC can include a low-powered processor for wireless communication (e.g., via BLE, Bluetooth, mesh near-field communication, Thread, Zigebee, etc.), general scheduling, and/or other operations where high efficiency is advantageous. The SOC can also include a higher-powered application processor for signal processing, onboard analysis, and/or other operations where high performance is advantageous. In other embodiments, however, other types of processors and configurations can be used.

The interface 386 can be or include an AFE or other analog interface for receiving and/or processing signals from the microneedle arrays 306a-c. The interface 386 can include circuitry for supporting a number of electrochemical techniques, including electrochemical impedance spectroscopy (EIS), cyclic voltammetry (CV), high frequency pulsed chronoamperometry, or the like. The interface 386 can optionally include or be coupled to multiplexer circuitry for rerouting electrical connections between the microneedle arrays 306a-c and other components of the electronics assembly 350 (e.g., the electrical contact 370) depending on the type of measurements being generated (e.g., analyte levels, bioimpedance, biopotential, ECG, etc.). Optionally, the electronics assembly 350 can include additional AFEs or other analog interfaces for receiving and/or processing signals from any of the other sensors of the device 300.

The memory unit(s) can include any suitable type of memory for buffering and/or storing data, such as flash memory, random access memory (RAM), cache memory, a first-in-first-out (FIFO) buffer, or combinations thereof. In some embodiments, the electronics assembly 350 includes multiple memory units, which can be at different locations of the device 300 and/or integrated into other components. For example, in embodiments where the processor(s) 376 are part of a SOC, the SOC can include integrated memory (e.g., flash memory and cache memory) associated with each processor 376. The electronics assembly 350 can also include a flash memory unit separate from the SOC. Additionally, any of the sensors described herein (e.g., optical sensor 362, ECG sensor, motion sensor 375, interface 386 for the microneedle arrays 306a-c, etc.) can include or be coupled to a respective memory unit (e.g., a FIFO buffer).

In some embodiments, the electronics assembly 350 is configured as a compact, foldable structure to reduce or minimize the overall size (e.g., volume, footprint) of the pod 304. This can be accomplished, for example, by distributing the components of the electronics assembly 350 across multiple substrates that can be folded, stacked, or otherwise arranged in close proximity with each other. In some embodiments, the electronics assembly 350 includes a primary substrate 390 (e.g., a first PCB) and a secondary substrate 392 (e.g., a second PCB). The primary substrate 390 can be a larger structure that carries most of the components of the electronics assembly 350, such as the pins 356, motion sensor 375, processor 376, memory unit, rechargeable battery 380, PMIC 382, and/or interface 386. The secondary substrate 392 can be a smaller structure that carries the optical sensor 362 and/or the temperature sensor 377.

FIGS. 3O and 3P are top and bottom views, respectively, of the electronics assembly 350 in an unfolded state (certain components of the electronics assembly 350 are omitted in FIGS. 3O and 3P for purposes of simplicity). As shown in FIGS. 3O and 3P, the primary substrate 390 can be mechanically and electrically coupled to the secondary substrate 392 via a first flex circuit 393. The primary substrate 390 can also be mechanically and electrically coupled to a second flex circuit 394 carrying the antenna 384. The second flex circuit 394 and antenna 384 can have a curved shape with a cutout to accommodate the electrical contact 370 (FIG. 3M). Optionally, the primary substrate 390 can be connected to a third flex circuit 395 providing an electrical connection to the rechargeable battery 380 (FIG. 3N).

Referring back to FIGS. 3K-3N, when the pod 304 is assembled, the secondary substrate 392 can be folded below the primary substrate 390 in a position partially or entirely within the protruding portion 364 of the lower housing 352b. The first flex circuit 393 can extend between the primary substrate 390 and the secondary substrate 392. The second flex circuit 394 can be folded above the primary substrate 390 so that the antenna 384 is located near or adjacent to the upper housing 352a. The third flex circuit 395 can be folded underneath the primary substrate 390 to cover at least a portion of the rechargeable battery 380.

The configuration of the electronics assembly 350 can be varied in many different ways. For example, in other embodiments, the components of the electronics assembly 350 can be at different locations on the primary substrate 390 and secondary substrate 392. The arrangement of the primary substrate 390, secondary substrate 392, and flex circuits 393-395 can also be altered. Optionally, the secondary substrate 392 and/or any of the flex circuits 393-395 can be omitted. The electronics assembly 350 can also include additional substrates, flex circuits, etc., not shown in the illustrated embodiments.

FIGS. 3Q and 3R are top and bottom perspective views, respectively, of the device 300 with a sealing element 396 and cover 397. Before use, the sealing element 396 and cover 397 can be temporarily attached to the patch 302 to protect the device components at the lower surface of the patch 302. For example, the sealing element 396 can be a film, sheet, tape, etc., that partially or fully covers the adhesive region 318 (obscured in FIGS. 3Q and 3R). Although FIG. 3R depicts the sealing element 396 as having multiple sections or leaflets, (e.g., first and second leaflets 398a-b), in other embodiments, the sealing element 396 can be a single unitary structure. The cover 397 can enclose some or all of the following components: the microneedle arrays 306a-c, temperature sensor 324, memory 326, test points 328, and/or windows 366 (these components are obscured in FIGS. 3Q and 3R). The cover 397 can protrude below the sealing element 396 to ensure sufficient clearance between the interior of the cover 397 and the microneedle arrays 306a-c (and/or other enclosed components).

The sealing element 396 (e.g., the first leaflet 398a) can include an elongate tab 399 extending outward away from the device 300. To prepare the device 300 for application to the skin, the user can grasp and pull the tab 399 to separate the sealing element 396 and cover 397 from the patch 302. In some embodiments, the cover 397 is coupled to the sealing element 396 such that the user can remove both of these components in a single step by simply by pulling on the tab 399. In other embodiments, however, the cover 397 may be removed from the device 300 separately from the sealing element 396. Alternatively, the cover 397 can be omitted, such that the sealing element 396 covers the entire lower surface of the patch 302.

### B. Biosensor Kits and Associated Devices and Methods

In some embodiments, the biosensors described herein are provided as part of a kit. The kit can include, for example, one or more disposable biosensor components (e.g., the patch 202 of FIG. 2 and/or the patch 302 of FIGS. 3A-3R) and one or more reusable biosensor components (e.g., the pod 204 of FIG. 2 and/or the pod 304 of FIGS. 3A-3R). For example, a kit can include a single reusable pod and multiple disposable patches (e.g., at least five, ten, 20, 30, 40, or 50 patches). Some or all of the patches can be of the same type (e.g., configured to detect the same analyte(s)), or some or all of the patches can be of different types (e.g., configured to detect different analyte(s)). Different patches can be used at different times (e.g., different days, weeks, months, etc.), depending on the disease or condition to be monitored, a recommendation or prescription from a healthcare professional, changes in the user's health state, the user's health goals, or any other suitable factor. As described above, the algorithms and/or signal processing parameters implemented by the pod can be adjusted, depending on the particular patch in use.

The kits described herein can include accessory devices for the biosensors disclosed herein. For example, although some embodiments of the biosensors described herein may be applied manually by the user (e.g., by pressing the pod and/or patch against the skin), in other embodiments, an applicator can be used to apply force to the biosensor to mount the biosensor on the user's body. An applicator can be advantageous, for example, in situations where relatively short microneedles are used (e.g., less than 1 mm in length), since such microneedles may need to be driven into the skin at a velocity greater than or equal to the viscoelastic response of the skin (e.g., at least 5 m/s) to ensure sufficient penetration. The appropriate driving velocity for the microneedles can depend on the length and/or spacing of the microneedles, as well as other parameters. Other accessory devices that may be provided as part of a kit include, but are not limited to, a pedestal for loading of the biosensor into the applicator, a charging station for recharging the power source of the biosensor, and/or any other devices that facilitate use and/or maintenance of the biosensor.

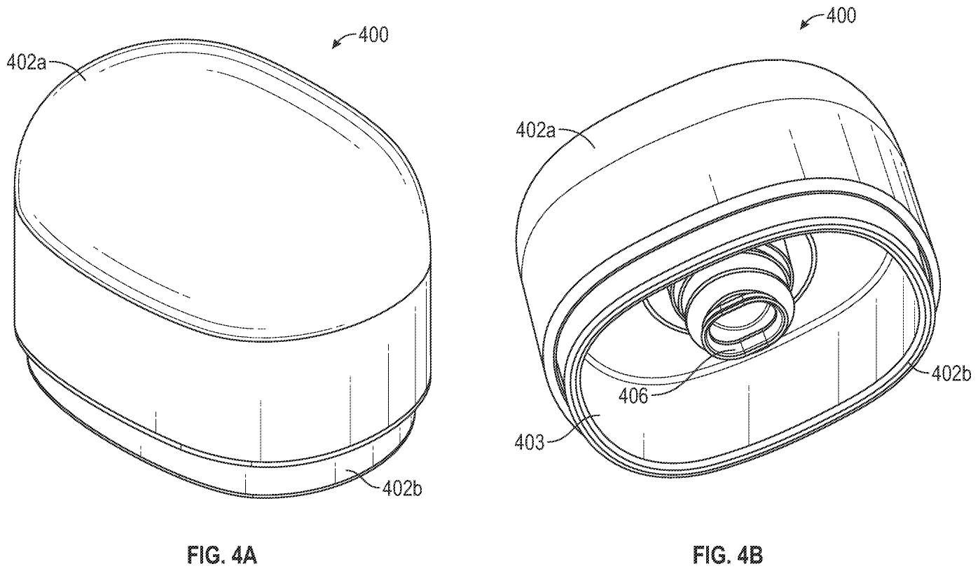

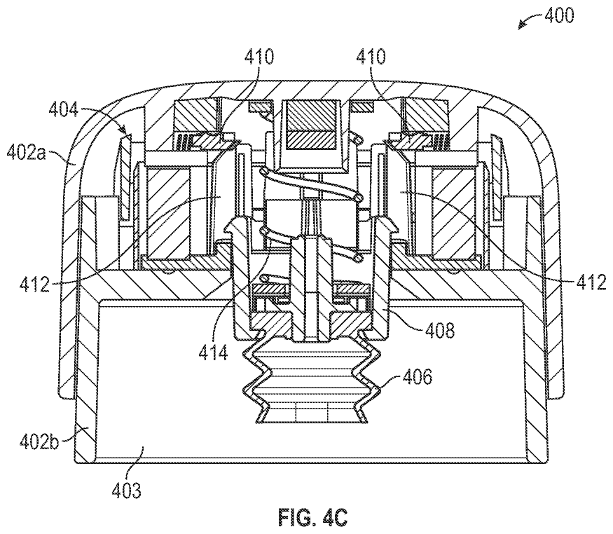

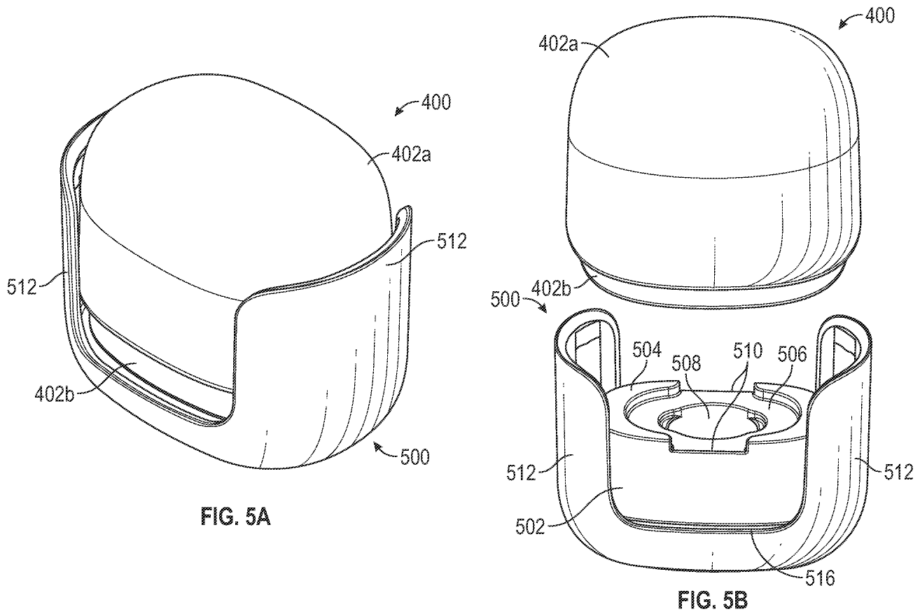

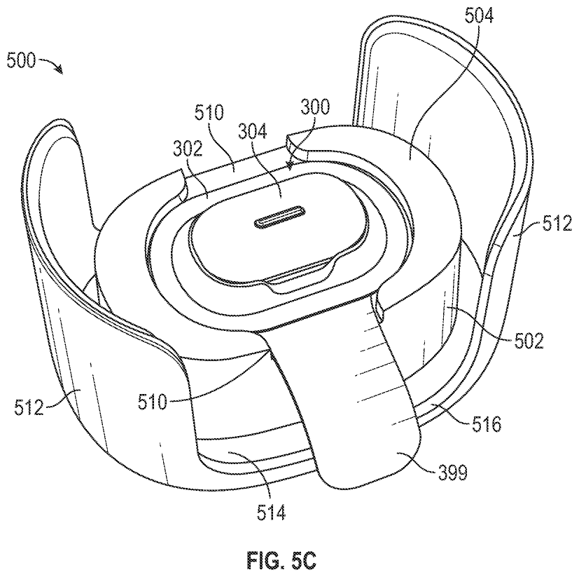

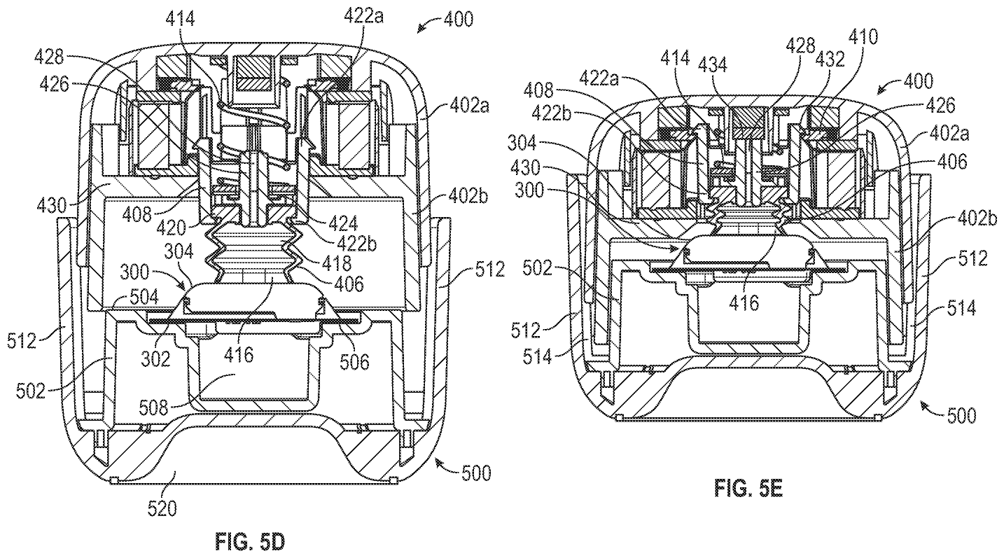

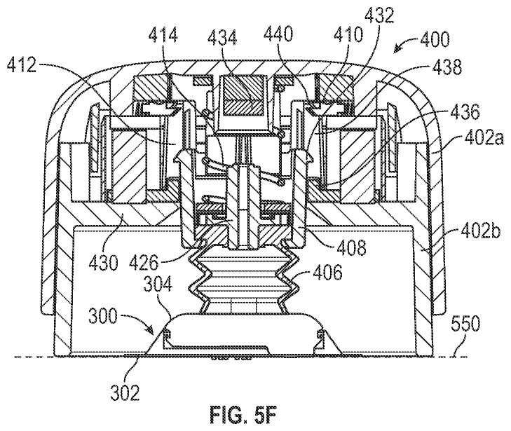

FIGS. 4A-5F illustrate an applicator 400 and a pedestal 500 configured in accordance with embodiments of the present technology. Specifically, FIGS. 4A-4C show various views of the applicator 400, FIGS. 5A and 5B show the applicator 400 together with the pedestal 500, FIG. 5C shows the device 300 of FIGS. 3A-3R on the pedestal 500, and FIGS. 5D-5F show the device 300, applicator 400, and pedestal 500 during operation. Although certain features of the applicator 400 and pedestal 500 are described herein in connection with the device 300 of FIGS. 3A-3R, this is merely for illustrative purposes, and the applicator 400 and pedestal 500 can be used with any embodiment of the biosensor devices described herein.

Referring first to FIGS. 4A (top perspective view of the applicator 400), 4B (bottom perspective view), and 4C (cross-sectional view) together, the applicator 400 can be manually operated by a user to apply a biosensor device (e.g., the device 300 of FIGS. 3A-3R) to the user's skin. The applicator 400 can include an upper applicator housing 402a (“upper housing 402a”) and a lower applicator housing 402b (“lower housing 402b”). The upper housing 402a can be a rounded, dome-like shell or structure having an interior cavity. The lower housing 402b can be a hollow, barrel- or tube-like structure that fits at least partially within the upper housing 402a. The lower housing 402b can include an opening 403 that is sufficiently large to fit over the biosensor. The upper and lower housings 402a-b can be slidably and/or telescopically coupled to each other so the upper housing 402a can move up and down relative to the lower housing 402b. The geometry and configuration of the upper and lower housings 402a-b can be varied as desired. For example, although the illustrated embodiment depicts the upper and lower housings 402a-b as each having a generally oval cross-sectional shape, in other embodiments, the upper and/or lower housings 402a-b can each have a different cross-sectional shape, such as a circular, rectangular, square, or other suitable shape. The upper and lower housings 402a-b can each be made of a durable material suitable for reuse, such as plastic.

Referring next to FIG. 4C, the upper and lower housings 402a-b can collectively enclose an actuation mechanism 404 for loading a biosensor and applying the biosensor to the user's skin (also referred to herein as “firing” the biosensor). In the illustrated embodiment, for example, the actuation mechanism 404 includes: a suction element 406 for temporarily coupling to the biosensor; a hammer 408 carrying the suction element 406 and movable between resting, loaded, and firing configurations; a set of latches 410 for temporarily securing the hammer 408 in the loaded configuration, a trigger 412 for releasing the hammer 408 from the loaded configuration, and a spring 414 for firing the hammer 408 and suction element 406 towards the user's skin. Additional features of the actuation mechanism 404, as well as the process for operating the actuation mechanism 404, are described in detail below with reference to FIGS. 5D-5F.

FIGS. 5A and 5B are top perspective and exploded views, respectively, of the applicator 400 of FIGS. 4A-4C together with a pedestal 500; and FIG. 5C is a top perspective view of the device 300 of FIGS. 3A-3R on the pedestal 500. The pedestal 500 can assist the user in loading the device 300 into the applicator 400, e.g., by facilitating proper alignment of the applicator 400 with the device 300 and/or providing a solid surface to support the device 300 during loading. In other embodiments, however, the device 300 can be loaded into applicator 400 without using the pedestal 500.

As best seen in FIG. 5B, the pedestal 500 includes a platform or base 502 for supporting the device 300. The platform 502 can be a rigid, durable structure made of plastic or other suitable material. The platform 502 can include an upper surface 504 having a recessed portion 506 for receiving the device 300. For example, the geometry (e.g., size, shape) of the recessed portion 506 can be similar to the geometry of the device 300, or at least a portion thereof (e.g., the patch 302). As shown in FIG. 5C, when the device 300 is placed in the recessed portion 506, the lateral sidewalls of the recessed portion 506 can contact the edges of the patch 302 to constrain the device 300 to a desired position and orientation. Optionally, the recessed portion 506 can include a hole or cavity 508 (FIG. 5B) extending into the interior of the platform 502. The hole 508 can be configured to accommodate components protruding from the lower surface of the device 300 (e.g., the microneedle arrays 306a-c, protruding portion 364, and/or cover 397—obscured in FIG. 5C), so that the remaining regions of the lower surface can lie flush on the recessed portion 506.

Referring next to FIGS. 5B and 5C together, the platform 502 can optionally include one or more cutouts 510 formed in the sidewalls of the recessed portion 506. The cutouts 510 can be U-shaped grooves, indentations, etc., configured to accommodate the tab 399 of the device 300. Accordingly, the tab 399 can extend outward over the edge of the platform 502 when the device 300 is placed in the recessed portion 506. Although the illustrated embodiment depicts the platform 502 with two cutouts 510 at opposite lateral sides of the recessed portion 506, in other embodiments, the platform 502 can include a different number of cutouts 510 (e.g., a single cutout), or the cutouts 510 can be omitted altogether.

Referring again to FIGS. 5A-5C together, the pedestal 500 can optionally include a set of guide structures 512 for assisting the user in aligning the applicator 400 with the platform 502 and device 300. In the illustrated embodiment, the guide structures 512 are configured as a pair of vertical panels or walls surrounding the platform 502. The guide structures 512 can be spaced apart from the lateral surfaces of the platform 502 so there is a gap 514 (FIG. 5C) between the guide structures 512 and the platform 502. Accordingly, to load the device 300 into the applicator 400, user can position the applicator 400 within the guide structures 512 and over the platform 502. The user can then push downward on the applicator 400 to slide the upper and lower housings 402a-b into the gap 514 around the platform 502. The downward movement of the upper and lower housings 402a-b can cause the applicator 400 to transition into a loaded configuration that engages the device 300, as described further below.

In the illustrated embodiment, the guide structures 512 are localized to the opposite ends of the pedestal 500, thus defining a pair of openings 516 that expose the lateral sides of the platform 502. As shown in FIG. 5C, the openings 516 can accommodate the tab 399 of the device 300. Accordingly, the tab 399 can extend outward from the pedestal 500 when the device 300 is placed on the platform 502. The openings 516 can be sufficiently deep such that at least a portion of the tab 399 is still accessible even when the applicator 400 is lowered completely onto the device 300. The geometry (e.g., size and/or shape) and locations of the openings 516 can be varied as desired. In the illustrated embodiment, for example, the pedestal 500 includes two U-shaped openings 516 located at opposite lateral sides of pedestal 500. In other embodiments, the pedestal 500 can include a different number of openings 516, the openings 516 can have different shapes and/or be at different locations, one or more both of the openings 516 can be omitted, etc.

FIGS. 5D-5F are side cross-sectional views of the applicator 400 and pedestal 500 during an operation to load and fire the device 300 (the details of the device 300 are omitted for purposes of clarity). Specifically, FIG. 5D illustrates the applicator 400 in a resting configuration, FIG. 5E illustrates the applicator 400 in a loaded configuration, and FIG. 5F illustrates the applicator 400 after firing.

Referring first to FIG. 5D, the device 300 is initially positioned on the platform 502 with the patch 302 within the recessed portion 506. The user can insert the applicator 400 within the guide structures 512, and over the platform 502 and device 300. The user can push the applicator 400 downward until the suction element 406 contacts the upper surface of the device 300. In the illustrated embodiment, the suction element 406 comes into direct contact with only the pod 304, and does not directly contact the patch 302. In other embodiments, however, the suction element 406 can directly contact the patch 302, with or without directly contacting the pod 304. The suction element 406 can be a bellows or other hollow structure made of a flexible and/or elastomeric material, such as silicone. The suction element 406 can have a distal opening 416 connected to an interior lumen 418, and a proximal end 420 opposite the distal opening 416. The applicator 400 can be moved toward the device 300 until the pod 304 contacts and seals the distal opening 416 of the suction element 406.

The suction element 406 can be coupled to the hammer 408. The hammer 408 can be a tubular structure including a proximal end 422a, distal end 422b, an internal disk 424 near the distal end 422b, and a venting tube 426 extending through the internal disk 424 and within the lumen of the hammer 408. The proximal end 420 of the suction element 406 can be connected to the distal end 422b and internal disk 424 of the hammer 408. The venting tube 426 can be inserted at least partially through the proximal end 420 of the suction element 406 so that the interior lumen 418 of the suction element 406 is connected to a venting channel 428 of the venting tube 426.

The spring 414 can extend between the hammer 408 and the interior surface of the upper housing 402a. In the illustrated embodiment, the spring 414 is coupled to the internal disk 424 and positioned around the venting tube 426. The hammer 408 can initially be positioned away from the upper housing 402a so that the spring 414 is a resting (e.g., uncompressed) configuration. For example, the hammer 408 can be positioned in an aperture formed in an internal plate 430 of the lower housing 402b, such that the proximal end 422a of the hammer 408 is located above the internal plate 430 and the distal end 422b is located below the internal plate 430.

Referring next to FIG. 5E, as the user continues pushing down on the applicator 400, the upper and lower housings 402a-b can slide downward in the gap 514 between the guide structures 512 and the platform 502. The downward force can compress the suction element 406 against the pod 304, and can also push the hammer 408 upward through the aperture in the internal plate 430 of the lower housing 402b and toward the upper housing 402a. In the illustrated embodiment, for example, the hammer 408 has been displaced upward so that its distal end 422b is located above the internal plate 430. The upward movement of the hammer 408 can compress the spring 414 against the upper housing 402a, thus placing the spring 414 into a loaded (e.g., compressed) configuration.

The hammer 408 can move upwards until a set of hooks 432 at the proximal end 422a of the hammer 408 engages the set of latches 410 near the upper portion of the upper housing 402a. The engagement between the latches 410 and the hooks 432 can produce an audible click or other sound to signal to the user that the applicator 400 is now loaded. The contact between the latches 410 and hooks 432 can prevent the hammer 408 from being driven downward by the spring 414. Each latch 410 can be spring-loaded so the latch 410 is biased inward to remain engaged with the hooks 432, thus maintaining the hammer 408 and spring 414 in the loaded configuration.

When the hammer 408 is in the loaded configuration, the venting tube 426 can be brought into contact with a seal block 434 connected to the interior surface of the upper housing 402a. Because the distal opening 416 of the suction element 406 is sealed by the pod 304, and the venting channel 428 of the venting tube 426 is sealed by the seal block 434, the pod 304 can be coupled to the suction element 406 by vacuum pressure. Accordingly, the device 300 is loaded in the applicator 400, such that the user can lift the device 300 away from the pedestal 500 simply by pulling the applicator 400 upward.

Referring next to FIG. 5F, before applying the device 300, the user can expose the lower adhesive surface of the patch 302 by removing the sealing element 396 and cover 397, if present (not shown in FIG. 5F). The user can then place the applicator 400 over the desired body region so that the lower housing 402b contacts the skin surface 550. At this point, because the applicator 400 is still in the loaded configuration, the device 300 can be suspended within the applicator 400 and above the skin surface 550.

To fire the applicator 400, the user can press down on the upper housing 402a, thus causing the upper housing 402a to slide downward relative to the lower housing 402b and toward the skin surface 550. As the upper housing 402a moves downward, the trigger 412 can contact and disengage the latches 410 the from hooks 432 of the hammer 408. In the illustrated embodiment, for example, the trigger 412 includes a base 436 mounted on the internal plate 430, and a plurality of elongate arms 438 extending upward from the base 436. When the upper housing 402a moves down relative to the lower housing 402b, the upper ends 440 of the arms 438 can contact the latches 410 (or another component carrying the latches 410) to push the latches 410 outward and away from the hooks 432.

Once the hooks 432 have been released from the latches 410, the spring 414 can revert back toward its resting (e.g., uncompressed) configuration. The force exerted by the spring 414 can fire the hammer 408 and suction element 406 downward, thus bringing the patch 302 of the device 300 into contact with the skin surface 550. The downward movement of the hammer 408 can separate the venting tube 426 from the seal block 434, thus breaking the vacuum against the pod 304 and allowing the device 300 to be released from the suction element 406. Thus, when the user lifts the applicator 400 upward and away from the skin surface 550, the device 300 can remain attached to the skin.

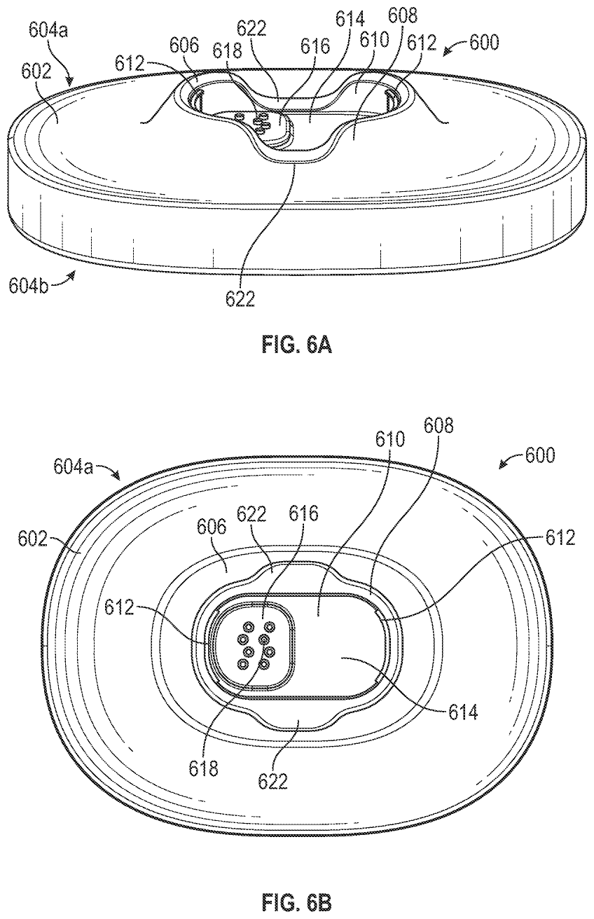

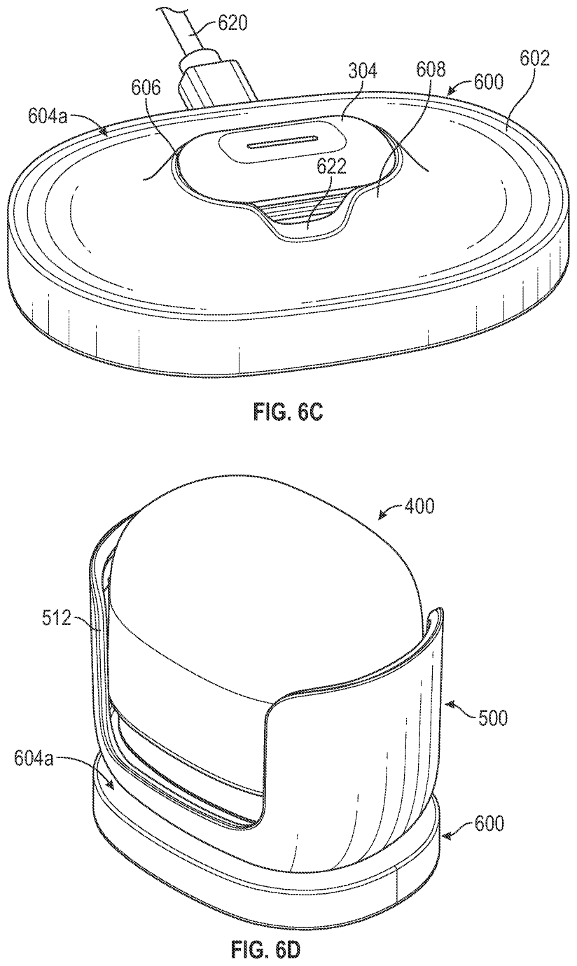

FIGS. 6A-6C illustrate a charging station 600 (“station 600”) configured in accordance with embodiments of the present technology. Although certain features of the station 600 are described herein in connection with the device 300 of FIGS. 3A-3R, this is merely for illustrative purposes, and the station 600 can be used with any embodiment of the biosensor devices described herein.

Referring first to FIGS. 6A (perspective view) and 6B (top view), the station 600 includes a base 602 including an upper surface 604a and a lower surface 604b. The base 602 can be a hollow structure that houses various electronic components (not shown), such as a PCB with power transmission and/or management circuitry, interfaces for connecting to an external power supply (e.g., a USB interface), control circuitry for status indicators (e.g., LEDs), and/or other suitable components for recharging and/or related operations. Although the base 602 is illustrated as having an oval shape, in other embodiments, the base 602 can have a different shape (e.g., circular, square, rectangular, etc.). The base 602 can be made of any suitable durable material, such as plastic. Optionally, the lower surface 604b of the base 602 (obscured in FIGS. 6A and 6B) can include a material to improve grip and avoid slipping, such as a polymer.

Referring next to FIGS. 6A-6C together, the upper surface 604a of the base 602 includes a docking receptacle 606 configured to receive and couple to the pod 304 of the device 300 (FIG. 6C). The docking receptacle 606 can include a set of raised walls 608 defining a cavity 610 shaped to releasably couple to the pod 304. In some embodiments, the inner surfaces of the walls 608 include a set of ridges 612 configured to engage the groove 340 of the pod 304 via snap fit, which may be identical or similar to the ridges 338 of the patch 302 described above in connection with FIGS. 3B and 3E. In other embodiments, however, the docking receptacle 60 can couple to the pod 304 using other techniques, such as interference fit, threading, fasteners, etc.

The docking receptacle 606 can include a bottom surface 614 including a raised region 616 having a plurality of electrical contacts (e.g., pin contacts 618). The height of the raised region 616 can be selected so that the protruding portion 364 (FIG. 3I) of the pod 304 is near or in contact with the bottom surface 614 while the pins 356 (FIG. 3I) of the pod 304 are in contact with the pin contacts 618. Each pin contact 618 can electrically couple to a corresponding pin 356 on the pod 304. Accordingly, power can be transmitted from the station 600 to the pod 304 via the connection between the pin contacts 618 and the pins 356. In some embodiments, the station 600 receives power from an external power supply (e.g., via a USB connector 620 or other wired or wireless connection), and relays the power to the pod 304. Alternatively or in combination, the station 600 can include its own internal power supply, which can be used to recharge the pod 304 even when the station 600 is not connected to an external power supply.

In some embodiments, the docking receptacle 606 includes a set of cutouts 622 formed in the walls 608 of the docking receptacle 606. Although the illustrated embodiment shows two U-shaped cutouts 622 located at opposite lateral sides of the docking receptacle 606. In other embodiments, the docking receptacle 606 can include a different number of cutouts 622, the cutouts 622 can have different shapes and/or be at different locations, etc. The geometry (e.g., size, shape) of the cutouts 622 can be configured to expose the lateral surfaces of the pod 304 to allow a user to manually separate the pod 304 from the docking receptacle 606, e.g., by gripping the exposed surfaces of the pod 304 with the fingers, by inserting a removal tool into the cutout 622 and between the walls 608 and the pod 304, etc.

Optionally, the station 600 can include electronic components (e.g., one or more processors, memory, and/or other circuitry) configured to detect an operational status of the pod 304. The operational status can include, for example, a charge level of the power source of the pod 304, whether the pod 304 is functioning properly, whether there are any errors or anomalies detected, the remaining lifetime of the pod 304, the calibration status of one or more sensors carried by the pod 304 (e.g., the interface 386 for the microneedles 306a-c, other sensor interfaces or modules), and the like. In some embodiments, the station 600 also includes components for calibrating one or more sensors of the pod 304. The station 600 can include at least one indicator configured to output signals representing the operational status of the pod 304. For example, the station 600 can include one or more light sources (e.g., LEDS—not shown) that may convey status information to the user by turning on or off, displaying different colors, flashing or blinking, etc.

In some embodiments, the pod 304 is configured to pair with a remote device, such as a mobile device, smartwatch, computer, or other user device. As discussed elsewhere herein, the pairing between the pod 304 and the remote device can allow the pod 304 to transmit data to and/or receive data from the remote device (e.g., sensor signals, health measurements, calibration or other signal processing parameters, software updates, control signals, etc.). To improve device security and/or prevent accidental pairing with other devices, the pod 304 can be configured so that pairing or changes in pairing can occur only when the pod 304 is coupled to the station 600. In such embodiments, the pod 304 can detect whether it is currently coupled to the station 600, e.g., based on signals received from the station 600 via the pin contacts 618. If so, the pod 304 can permit pairing with a remote device and/or switch to pairing with a new device, in accordance with techniques known to those of skill in the art. If the pod 304 detects that it is not coupled to the station 600, the pod 304 can prevent pairing with any remote device and/or prevent changes from the current pairing. In other embodiments, however, the pairing of the pod 304 with a remote device can be performed regardless of whether the pod 304 is coupled to the station 600.

FIG. 6D is a perspective view of the station 600 together with the applicator 400 and pedestal 500 of FIGS. 4A-5F. As shown in FIG. 6D, the applicator 400, pedestal 500, and station 600 can be assembled into a compact unit, e.g., for storage. For example, the applicator 400 can be placed over the platform 502 of the pedestal 500 (obscured in FIG. 6D) and within the guide structures 512 of the pedestal 500. The pedestal 500 can be placed on the upper surface 604a of the station 600. Optionally, as shown in FIG. 5C, the base of the pedestal 500 can include a recess or cavity 520 configured to receive the docking receptacle 606 of the station 600. The recess 520 can also be sufficiently large to accommodate a pod 304 coupled to the docking receptacle 606.

### III. MICRONEEDLES, MICRONEEDLE ARRAYS, AND ASSOCIATED METHODS

In some embodiments, the biosensors described herein use one or more microneedles (e.g., a plurality of microneedles arranged in one or more arrays) to detect analyte levels in the skin and/or other health parameters. The microneedles can be configured to penetrate into the user's skin to access interstitial fluid therein. In some embodiments, the microneedles are configured to penetrate only into the stratum corneum and epidermis, and do not extend into the dermis or hypodermis (subcutaneous tissue). This approach can reduce or avoid pain and/or discomfort, while still providing accurate detection of analytes in the epidermal interstitial fluid. In such embodiments, the microneedles can each have a length less than or equal to 500 μm, 475 μm, 450 μm, 425 μm, 400 μm, 350 μm, 300 μm, 250 μm, 200 μm, 150 μm, 100 μm or 50 μm. In other embodiments, however, the microneedles can be configured to access the dermis and/or the hypodermis (e.g., the microneedles can have a length greater than or equal to 500 μm, 1000 μm, 2000 μm, 3000 μm, 4000 μm, or 5000 μm).

The microneedles described herein can be configured to detect one or more analytes in the interstitial fluid, such as glucose, gases, electrolytes, BUN, creatinine, ketones, alcohols, amino acids, neurotransmitters, hormones, biomarkers, drugs, pH, cell count, and/or any of the other analytes described herein. Each microneedle can be configured to detect a single analyte, or some or all of the microneedles can be configured to detect multiple analytes (e.g., two, three, four, five, or more different analytes). In some embodiments, the microneedles are solid structures configured to detect analytes via interactions with one or more functional layers on the surfaces of the microneedles (e.g., electrochemical reactions), rather than hollow structures including fluidic channels, openings, etc., for receiving and drawing fluid into the interior of the microneedles. In other embodiments, however, some or all of the microneedles can be configured to detect analytes via fluidic channels, openings, etc. Optionally, some or all of the microneedles can be configured to detect other health parameters, such as electrical properties (e.g., biopotential, bioimpedance), physiological parameters (e.g., body temperature), etc.

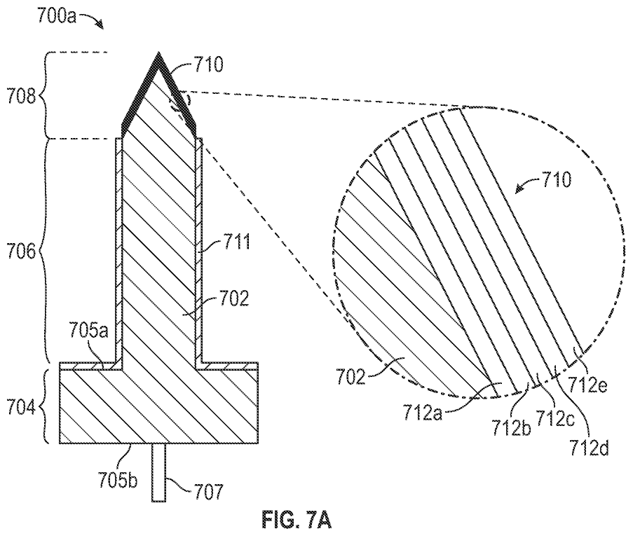

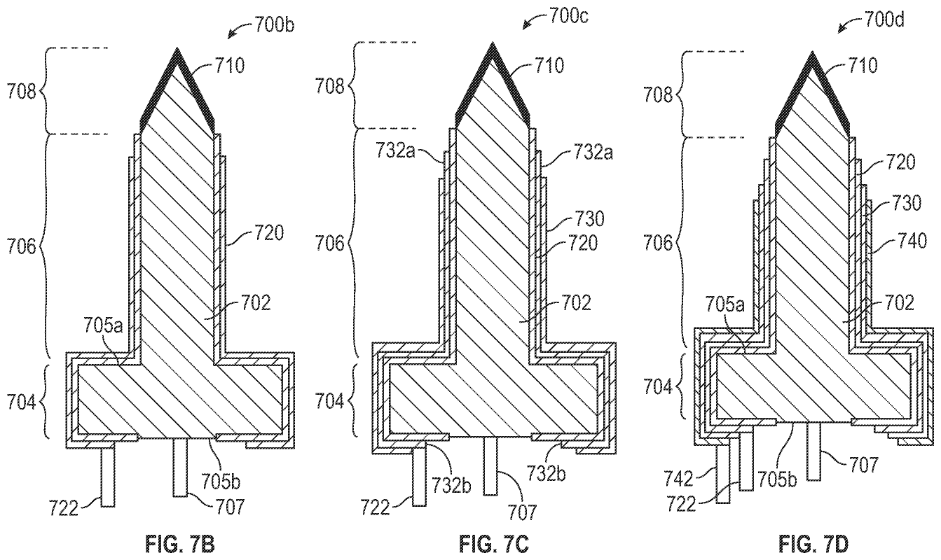

FIGS. 7A-7D are partially schematic illustrations of microneedles 700a-d configured in accordance with embodiments of the present technology. Any of the features of the microneedles 700a-d can be incorporated in any embodiment of the biosensors described herein (e.g., the device 200 of FIG. 2, the device 300 of FIGS. 3A-3R). Referring first to FIG. 7A, the microneedle 700a includes a substrate 702 having a base 704, and a needle body 706 extending from the base 704. The substrate 702 can be composed of a semiconducting material (e.g., silicon, quartz, gallium arsenide), a conducting material (e.g., gold, steel, platinum, nickel, silver, polymer, etc.), and/or an insulating or non-conductive material (e.g., glass, ceramic, polymer, etc.). In some embodiments, the substrate 702 can include a combination of materials (e.g., a composite, alloy, etc.).

The needle body 706 can be an elongate protrusion or column connected to a front side 705a of the base 704. The needle body 706 can have any suitable cross-sectional shape or profile, such as square, rectangular, triangular, circular, oval, polygonal, non-polygonal, etc. The needle body 706 can terminate in a tip 708 configured to penetrate into the skin. As shown in FIG. 7A, the tip 708 can be a sharpened structure having a multi-faceted (e.g., pyramidal) or other suitable shape. The needle body 706 and tip 708 can collectively have a length less than or equal to 500 μm, 475 μm, 450 μm, 425 μm, 400 μm, 350 μm, 300 μm, 250 μm, 200 μm, 150 μm, 100 μm or 50 μm (or any other suitable length).

In some embodiments, the microneedle 700a is a solid, continuous structure that lacks any openings, channels, pores, etc., for transporting fluid into the interior of the substrate 702. Accordingly, the microneedle 700a can be configured to operate without microfluidics, reagent solutions, and/or other fluid-based analyte detection mechanisms. Instead, the microneedle 700a can detect analytes using one or more material layers on the surface of the substrate 702, which can reduce the number of components required and simplify sensor manufacturing and operation. The microneedle 700a can include a sensing or active region 710 configured for analyte detection. The sensing region 710 can generate electrical signals upon detection of one or more target analytes. The signals can be transmitted by the substrate 702 through the needle body 706 to the base 704, and subsequently to a set of electrical contacts 707 (e.g., a conductive interconnect, bond pad, or other circuitry) connected to a back side 705b of the base 704.

The remaining surfaces of the microneedle 700a can be passivated or otherwise covered by an insulating layer 711. The insulating layer 711 can be made of one or more non-conductive materials, such as an insulating polymer (e.g., polyimide, cyanate ester, polyurethane, silicone), an oxide, a carbide, a nitride (e.g., silicon nitride), or a combination thereof. The insulating layer 711 can be formed using any suitable technique, such as thermal oxidation, chemical vapor deposition, plasma-enhanced chemical vapor deposition, low pressure chemical vapor deposition techniques, dip coating, spray coating, and/or evaporation.

In the illustrated embodiment, the sensing region 710 is localized to the tip 708 of the microneedle 700a, and the remaining portions of the microneedle 700a (e.g., the needle body 706 and/or base 704) are covered by the insulating layer 711. Accordingly, analyte detection can occur only at the tip 708, which can improve sensor performance. For example, this configuration can improve accuracy and/or reduce calibration requirements, since the sensing region 710 is a well-defined surface area that is completely in contact with the interstitial fluid in the skin. This approach can also reduce the susceptibility of the sensor signal to leakage currents, electrical noise, non-specific electrochemical reactions, and/or noise or contamination from sweat and other surface contaminants. In other embodiments, however, the sensing region 710 can be located at a different portion of the microneedle 700a, the microneedle 700a can include multiple discrete sensing regions 710 at different locations, and/or the insulating layer 711 can be omitted.

The sensing region 710 can include a plurality of functional layers 712a-e (collectively, “layers 712”). The layers 712 can include, for example, a conductive layer 712a, a first barrier layer 712b, a reactive layer 712c, a second barrier layer 712d, and a protective layer 712e. The conductive layer 712a can provide a base electrochemical surface or material for facilitating electron transfer to the substrate 702, thus producing an electrical signal that can be transported by the needle body 706 to the base 704, and subsequently to coupled detection circuitry (not shown). For example, the conductive layer 712a can transfer electrons from one or more intermediate electroactive species generated by the other layers 712 to the underlying substrate 702. Alternatively, the conductive layer 712a may not transfer electronics, and may instead act as a conductive surface for non-faradaic processes. The conductive layer 712a can include any suitable electrically conductive material, such as platinum, palladium, iridium, tungsten, titanium, gold, silver, nickel, glassy carbon, silicon, doped silicon, or combinations thereof (e.g., a combination of titanium and platinum). In embodiments where multiple conductive materials are used, the materials can be combined into a single layer, can be sequentially deposited as discrete sublayers, or any other suitable configuration. Optionally, the conductive layer 712a (or a portion thereof, such as a titanium sublayer) can also a serve as an adhesion layer to enhance mechanical coupling of the sensing region 710 to the underlying substrate 702.

The first barrier layer 712b can be a selective transport membrane, diffusion barrier, or similar structure configured to restrict non-target chemical species from reaching the conductive layer 712a. The non-target species can include, for example, species that may foul the conductive layer 712a, generate a false signal from interacting with the conductive layer 712a, or produce any other activity that may interfere with analyte detection. The first barrier layer 712b can be configured to exclude non-target species based on size, charge, phase, hydrophobicity, atomic orbital structure, and/or any other suitable structure. Alternatively or in combination, the first barrier layer 712b can control the rate of transport of species to the conductive layer 712a. In some embodiments, the first barrier layer 712b includes a polymer, such as polytetrafluoroethylene (PTFE), polyethylene glycol (PEG), urethane, polyurethane, cellulose acetate, polyvinyl alcohol (PVA), polyvinyl chloride (PVC), polydimethylsiloxane (PDMS), parylene, polyvinyl butyral (PVB), a sulfonated tetrafluoroethylene, a chlorinated polymer, a fluorinated polymer, or suitable materials known to those of skill in the art or combinations thereof. Optionally, the first barrier layer 712b can include functional compounds such as lipids, charged chemical species, etc., that can provide a barrier against transport of non-target species.

The reactive layer 712c (also referred to herein as a “sensing layer”) can include one or more agents (e.g., enzymes, catalysts, conductive polymers, redox mediators, electron transporters, etc.) configured to facilitate a reaction with a target analyte to produce a chemical species that can be detected by the conductive layer 712a, referred to herein as an “intermediate species” or “mediator species.” For example, the agent can modify the target analyte to create the intermediate species, or can react with the analyte to produce a product that serves as the intermediate species. The reactive layer 712c can include a single agent (e.g., a single enzyme or catalyst), or can include multiple agents (e.g., two, three, four, five, or more different enzymes or catalysts). The agent can be selected based on the particular analyte or analytes to be detected. For example, the agent can be configured to react and/or interact with any of the analytes described herein, such as glucose, gases (e.g. oxygen, carbon dioxide, etc.), electrolytes (e.g., bicarbonate, potassium, sodium, magnesium, chloride, lactic acid, ascorbic acid), BUN, creatinine, ketones, cholesterol, triglycerides, alcohols, amino acids (e.g., glutamate, choline, tyrosine), neurotransmitters, hormones, disease biomarkers (e.g., cancer biomarkers, cardiovascular disease biomarkers), drugs, or combinations thereof.

The agent can be or include any suitable enzyme or catalyst known to those of skill in the art, such as an oxidoreductase, transferase, hydrolase, lysase, etc. Examples of enzymes or catalysts suitable for use in the reactive layer 712c can include, but are not limited to: glucose oxidase, creatine amidinohydrolase, alcohol oxidase, D- and L-amino acid oxidases, cholesterol oxidase, galactose oxidase, and urate oxidase. The agent can be configured to modify and/or react with a target analyte to produce any suitable intermediate species, such as hydrogen peroxide, ammonia, nicotinamide adenine dinucleotide (NAD), nicotinamide adenine dinucleotide phosphate (NADPH), flavin adenine dinucleotide (FAD), oxygen, or other small molecules. In some embodiments, the agent is embedded in, cross-linked to, and/or otherwise coupled to a matrix or membrane, such as a polymer matrix or membrane. The matrix or membrane can include any of the following: an aziridine-based polymer (e.g., polyethyleneimine), an amine-decorated polymer, polyethylene, PTFE, urethane, polyurethane, phenylenediamine, ortho-phenylenediamine, meta-phenylenediamine, tyramine, a protein matrix, an amino acid matrix, a crosslinker, other electropolymerized components, etc.

The second barrier layer 712d can be a selective transport membrane, diffusion barrier, or similar structure configured to restrict non-target species from reaching the reactive layer 712c. The non-target species can include, for example, species that may foul the reactive layer 712c, generate a false signal from interacting with the reactive layer 712c, or produce any other activity that may interfere with analyte detection. The second barrier layer 712d can be configured to exclude non-target species based on size, charge, phase, hydrophobicity, atomic orbital structure, and/or any other suitable structure. Alternatively or in combination, the second barrier layer 712d can control the rate of transport of species to the reactive layer 712c. The second barrier layer 712d can include any of the materials described above in connection with the first barrier layer 712b.

The protective layer 712e can be configured to protect the lower layers 712 from damage, such as mechanical damage and/or damage from cells, protein aggregation, biofouling, and/or enzymatic degradation. Alternatively or in combination, the protective layer 712e can improve biocompatibility, e.g., by providing anti-microbial and/or anti-inflammatory properties. The protect layer 712e can be made of any suitable material, such as PTFE, PEG, urethane, polyurethane, cellulose acetate, PVA, PVC, PDMS, parylene, PVB, a sulfonated tetrafluoroethylene, a chlorinated polymer, a fluorinated polymer, or a combination thereof. In some embodiments, the protective layer 712e is localized to the tip 708 of the microneedle 700a. In other embodiments, the protective layer 712e can extend over other portions of the microneedle 700a, such as over the needle body 706 and/or the base 704. In such embodiments, the protective layer 712e can be the outermost layer on the microneedle 700a (e.g., the protective layer 712e is positioned over the insulating layer 711 and/or any other layers over the insulating layer 711).

The configuration of the sensing region 710 can be modified in many different ways. For example, although the illustrated embodiment includes five layers 712, in other embodiments, the sensing region 710 can include a different number of layers 712 (e.g., one, two, three, four, six, seven, eight, nine, ten, or more layers 712). Any of the layers 712 can be divided into individual sublayers, or can be combined with each other into a single layer. The ordering of the layers 712 can also be varied. Additionally, the sensing region 710 can include additional functional layers not shown in FIG. 7A. In some embodiments, one or more of the layers 712 are optional and can be omitted. For example, in other embodiments, the second barrier layer 712d can be omitted, such that the sensing region 710 includes only the conductive layer 712a, first barrier layer 712b, reactive layer 712c, and protective layer 712e.

As another example, the reactive layer 712c and the second barrier layer 712d can be omitted, such that the sensing region 710 includes only the conductive layer 712a, first barrier layer 712b, and protective layer 712e. This configuration can be used, for example, for amperometric and/or potentiometric detection of analytes. In some embodiments, an amperometric detection scheme is used to detect oxygen, dissolved gases, and/or other small molecules. In such embodiments, the first barrier layer 712b can include one or more polymers, protein aggregates, metals, dielectrics and/or other materials having selective transport properties for the analyte of interest. A potentiometric detection scheme can be used to detect charged species such as ions (e.g., potassium, sodium, magnesium, chloride, metals), pH, and/or larger charged molecules. The first barrier layer 712b can include one or more polymers, protein aggregates, metals, dielectrics and/or other materials having selective transport properties for the charged species. Alternatively or in combination, the first barrier layer 712b can include chelating complexes for creating specificity for a target ion or metal. The complexes can be incorporated into the first barrier layer 712b via any suitable technique, such as entanglement, direct conjugation, hydrogen bonding, ionic interaction, and/or adsorption.

In a further example, the first barrier layer 712b and the reactive layer 712c can be omitted, such that the sensing region 710 includes the conductive layer 712a, second barrier layer 712d, and protective layer 712e; and a binding layer (not shown) can be added to the sensing region 710 between the conductive layer 712a and the second barrier layer 712d. This configuration can be used to detect nucleic acids (e.g., DNA or RNA oligomers), proteins, peptides, or other small molecules. Such analytes can be detected based on charge, surface capacitance, blocking transport, a conformational change activating a redox probe, or any other suitable probe.

In such embodiments, the binding layer can include a membrane, matrix, etc., having selective binding, adhesion, adsorption, and/or other interaction properties with the target analyte. This can be achieved, for example, through molecular engineering of the surface properties and/or manipulation of properties such as charge, viscoelastic properties, surface energy, hydrophobicity, surface roughness, topological morphology, or other general properties. Specificity can also be achieved by adding additional molecules, proteins, oligomers, coordination complexes, or polymers that bind specific molecules using a binding site or series of binding sites. Any of these binding and/or adhesion mechanisms may be reversible or irreversible, depending on the use case for the biosensor. The molecular association may change the surface properties of the binding layer above the conductive layer 712a resulting in a detectable change in the molecular microenvironment, including, but not limited to, changes in pH, charge, surface capacitance, hydration, or diffusion and transport properties. Alternatively or in combination, the association may induce specific conformation changes in the either the receptor or the analyte that result in a change of function or property of the either analyte or the complex. These changes can include conformation changes that produce any of the following results: bring a functional group or probe closer or further from the conductive layer 712a, a change in charge, a shifting of the energy level of electrons, and/or molecular orbitals within specific functional groups of either the receptor or analyte. These changes can be detected using stationary or dynamic electrochemical techniques including, but not limited to, cyclic voltammetry, pulsed voltammetry, electrochemical impedance spectroscopy, chronoamperometry, or chronopotentiometry.

FIGS. 7B-7D illustrate additional examples of microneedles 700b-d. The microneedles 700b-d can be generally similar to the microneedle 700a of FIG. 7A. Accordingly, like numbers indicate identical or similar components, and the discussion of the microneedles 700b-d will be limited to those features that differ from the microneedle 700a of FIG. 7A.

Referring first to FIG. 7B, the microneedle 700b includes a sensing region 710 localized to the tip 708 of the microneedle 700b, and an insulating layer 711 covering the needle body 706 and base 704. The microneedle can further include a conductive film 720, such as an evaporated metal film, or other conductive coating, layer, or material. As shown in FIG. 7B, the conductive film 720 is located on the insulating layer 711, and extends over at least a portion of the needle body 706 and base 704. The conductive film 720 can extend around to the back side 705b of the base 704 for connecting to a set of electrical contacts 722 located at or near the back side 705. In such embodiments, the insulating layer 711 can also extend to the back side 705b to avoid shorting between the conductive film 720 and the substrate 702. Alternatively, the conductive film 720 can terminate at the front side 705a of the base 704, and can be connected to the electrical contacts 722 using vias or other connectors extending between the front and back sides 705a-b of the base 704. The electrical contacts 722 for the conductive film 720 can be electrically isolated from the electrical contacts 707 for the sensing region 710 by an insulating material (not shown), thus providing two independent signal pathways. The configuration illustrated in FIG. 7B can be used to support a multi-analyte detection scheme in which the sensing region 710 serves as a working electrode, while the conductive film 720 serves as a reference electrode or counter electrode. This approach can eliminate the need for a separate reference electrode or counter electrode microneedle array.

Referring next to FIG. 7C, the microneedle 700c is generally similar to the microneedle 700b of FIG. 7B, except that microneedle 700c includes a second insulating layer 730 over the conductive film 720. The second insulating layer 730 can cover certain regions of the conductive film 720, while leaving selected regions exposed (e.g., upper regions 732a and/or lower regions 732b). The second insulating layer 730 can be or include any suitable passivating and/or insulating material, such as an insulating polymer (e.g., polyimide, cyanate ester, polyurethane, silicone), an oxide, a carbide, a nitride (e.g., silicon nitride), or a combination thereof. The configuration shown in FIG. 7C can protect the conductive film 720, and/or can prevent electrical noise or other conductive paths from interacting with the conductive films 720 except in the regions 732a-b where the conductive film 720 is exposed.

Referring next to FIG. 7D, the microneedle 700d is generally similar to the microneedle 700c of FIG. 7C, except that microneedle 700d includes a second conductive film 740 over the second insulating layer 730. The second conductive film 740 can be or include an evaporated metal film, or other conductive coating, layer, or material. The second conductive film 740 can be located on the second insulating layer 730, and can extend over at least a portion of the needle body 706 and base 704. The second conductive film 740 can extend around to the back side 705b of the base 704 for electrical coupling to a set of electrical contacts 742. Alternatively, the second conductive film 740 can terminate at the front side 705a of the base 704, and can be connected to the electrical contacts 742 using vias or other connectors extending between the front and back sides 705a-b of the base 704. The electrical contacts 742 for the second conductive film 740 can be electrically isolated from the electrical contacts 707 for the sensing region 710 and from the electrical contacts 722 for the conductive film 720 by an insulating material (not shown), thus providing three independent signal pathways. Optionally, the microneedle 700d can further include a third insulating layer (not shown) over the second conductive film 740, e.g., to protect the second conductive film 740 and/or reduce noise, similar to the function of the second insulating layer 730.

The configuration illustrated in FIG. 7D can be used to support a multi-analyte detection scheme in which sensing region 710 serves as a working electrode, the conductive film 720 serves as a reference electrode or counter electrode, and the second conductive film 740 serves as a second reference electrode or counter electrode. For example, the conductive film 720 can serve as the reference electrode, and the second conductive film 740 can serve as the counter electrode. This approach can eliminate the need for separate reference electrode and counter electrode microneedle arrays. Alternatively, one or both of the conductive films 720, 740 can be as blank electrodes, such as for tracking drift and/or for generating electrochemical impedance measurements (e.g., to detect changes in the epidermal microenvironment, to detect proper application and/or insertion of the microneedle 700d, etc.). Additionally, one or both of the conductive films 720, 740 can be used to detect another analyte (e.g., oxygen or any number of other molecules), e.g., in situations where simultaneous detection would be beneficial for calibration, sensor data fusion, multi-analyte measurements, etc.

Optionally, the configuration of FIG. 7D can be further modified to include additional conductive films interspersed with insulating layers, thus providing even more independent signal pathways in a single microneedle 700d. For example, the microneedle 700d can be modified to include a total of four, five, six, seven, eight, nine, ten, or more independent pathways. Each pathway can be connected to a respective set of electrical contacts. Accordingly, a single microneedle 700d can simultaneously include multiple electrodes, some or all of which can perform different functions (e.g., serve as working electrodes, reference electrodes, counter electrodes, blank electrodes, etc.).

Any of the microneedles described herein (e.g., the microneedles 700a-d of FIGS. 7A-7D) can be incorporated into a microneedle array. An array of microneedles can include any suitable number of microneedles, such as one, two, three, four, five, six, seven, eight, nine, 10, 15, 20, 25, 30, 35, 40, 45, 50, 60, 70, 80, 90, or 100 microneedles. The microneedles can be arranged in any suitable geometry (e.g., square, rectangular, circular, elliptical, etc.). For example, the microneedles can be arranged in a grid, such as a 2×2, 3×3, 4×4, 5×5, 6×6, 7×7, 8×8, 9×9, or 10×10 square grid. The grid can have any suitable spacing or pitch between individual microneedles, such as a spacing of at least 100 μm, 200 μm, 300 μm, 400 μm, 500 μm, 600 μm, 700 μm, 800 μm, 900 μm, or 1000 μm.

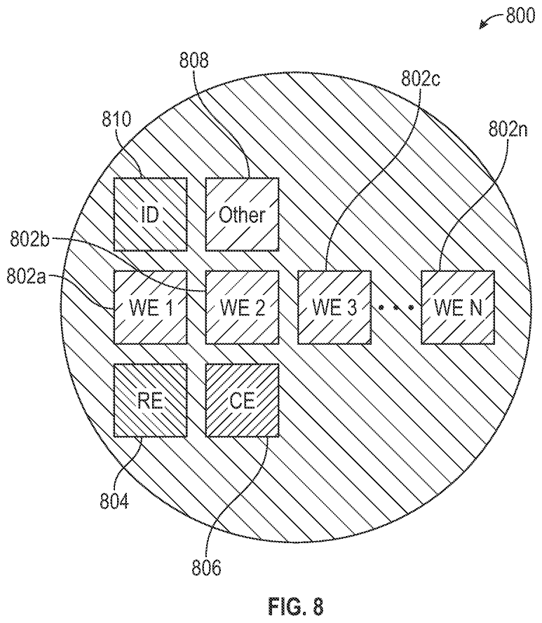

FIG. 8 is a schematic illustration of a biosensor 800 including multiple microneedle arrays in accordance with embodiments of the present technology. The features of the biosensor 800 can be incorporated in any of the other biosensors described herein (e.g., the device 200 of FIG. 2, the device 300 of FIGS. 3A-3R). Additionally, the microneedle arrays of the biosensor 800 can incorporate any of the microneedles described herein (e.g., the microneedles 700a-d of FIGS. 7A-7D) and/or can include any of the microneedle array features disclosed elsewhere herein.

As shown in FIG. 8, the biosensor 800 includes a set of first microneedle arrays 802a-n (e.g., collectively, “first microneedle arrays 802”) configured as working electrodes (“WE 1,” “WE 2,” etc.), a second microneedle array 804 configured as a reference electrode (“RE”), and a third microneedle array 806 configured as a counter electrode (“CE”). The properties of the microneedle arrays 802, 804, 806 (e.g., number, types, lengths, and/or arrangement of the microneedles used; composition and/or configuration of the layers on the microneedles; bias potential; etc.) can be varied based on the function to be performed.

The working electrodes of the biosensor 800 can be configured to perform analyte detection, in accordance with techniques described elsewhere herein. The number of first microneedle arrays 802a-n (and thus, the number of working electrodes) can be varied as desired. For example, the biosensor 800 can include one, two, three, four, five, or more first microneedle arrays 802, corresponding to one, two, three, four, five, or more working electrodes, respectively. A biosensor with a single working electrode can be used to detect a single set of analytes, whereas a biosensor with two or more working electrodes can be used to detect a single set of analytes or multiple analytes. For example, some or all of the first microneedle arrays 802 can detect the same set of analytes, which can improve accuracy and/or reliability of the measurement, as well as provide redundancy in case of sensor failure or other anomalies. Some or all of the first microneedle arrays 802 can detect different sets of analytes. In some embodiments, some or all of the first microneedle arrays 802 can have different microneedle lengths, e.g., if the analytes to be detected are located at different depths in the skin. Optionally, one or more of the first microneedle arrays 802 can be blank electrodes used for drift correction, etc.

The reference and counter electrodes can be auxiliary electrodes that are used to set the potential and source the current for the working electrodes, respectively. Although FIG. 8 depicts a single reference electrode and counter electrode, in other embodiments, the biosensor 800 can include multiple reference electrodes (multiple second microneedle arrays 804) and/or multiple counter electrodes (e.g., multiple third microneedle arrays 806).

In some embodiments, the biosensor 800 includes one or more other sensors 808 (“Other”), such as temperature sensors, optical sensors, bioimpedance sensors, biopotential sensors, ECG sensors, accelerometers, gyroscopes, and/or any of the other sensor types described herein. Optionally, the biosensor 800 can also include an identifier module 810 (“ID”), which can include a programmable memory storing information such as: the types of the microneedle arrays 802, 804, 806; the types of analytes detected by the first microneedle arrays 802; the configuration of the microneedle arrays 802, 804, 806; the types of the other sensors 808; a sensor ID; a lot ID; manufacturing date; expiration date; and/or any other suitable information, as described elsewhere herein.

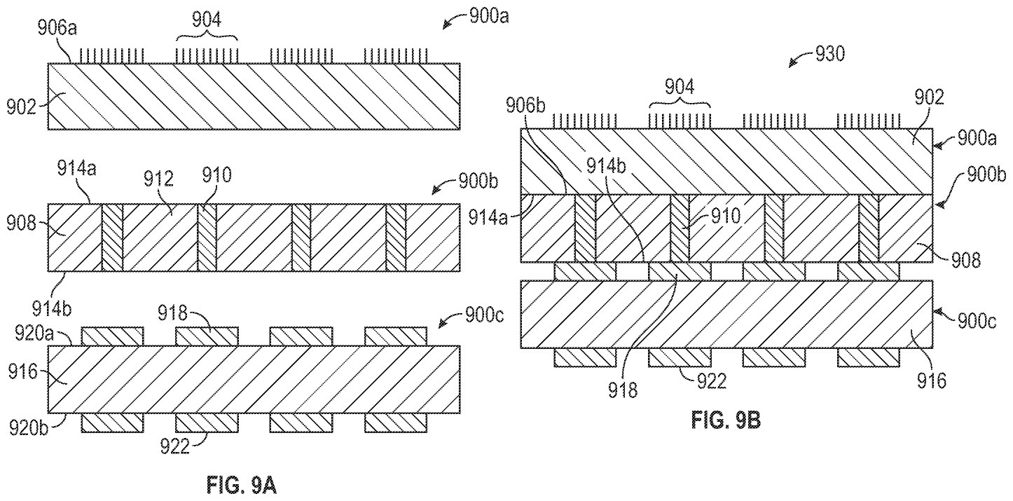

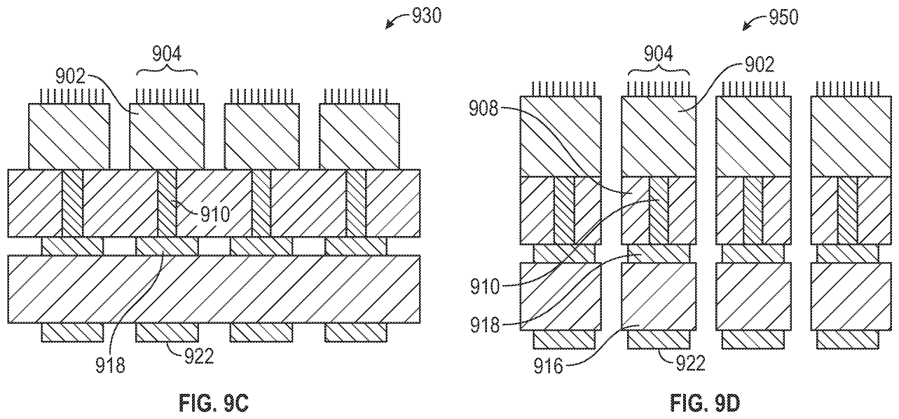

FIGS. 9A-9D are schematic illustrations of various stages of a method or process for manufacturing microneedle arrays, in accordance with embodiments of the present technology. The method illustrated in FIGS. 9A-9D can be used to manufacture any embodiment of the microneedle arrays described herein. Referring first to FIG. 9A, a first stage of the method includes manufacturing three separate subassemblies 900a-c. The first subassembly 900a can include a first substrate 902 (e.g., a silicon wafer or substrate) supporting a plurality of microneedle arrays 904. The microneedle arrays 904 can be patterned or otherwise formed on a front side 906a of the first substrate 902 using microfabrication techniques known to those of skill in the art. Example techniques for manufacturing microneedles and/or microneedle arrays are described in U.S. Pat. Nos. 10,173,042 and 10,820,860, which are incorporated by reference herein in their entireties.

The second subassembly 900b can include a second substrate 908 (e.g., a silicon wafer or substrate) including a plurality of conductive interconnects 910 separated by non-conductive regions 912 (e.g., regions made of insulating materials). The conductive interconnects 910 can be or include vias, traces, routing, etc., that providing an electrical path from a front side 914a of the second substrate 908 to a back side 914b of the second substrate 908. Each conductive interconnect 910 depicted in FIG. 9A can represent a single connection, or can represent multiple independent electrical connections (e.g., for use with more complex microneedle configurations, such as microneedles including multiple conductive films or layers).

The third subassembly 900c can include a third substrate 916 (e.g., a silicon wafer or substrate) including a plurality of first electronic modules or units 918. The first electronic modules 918 can be located on a front side 920a of the third substrate 916. Optionally, the third subassembly 900c can also include a plurality of second electronic modules 922 on a back side 920b of the third substrate 916. In such embodiments, the third substrate 916 can include vias, traces, routing, etc., providing an electrical path between each first electronic module 918 and second electronic module 922. Each electronic module 918, 922 can include various electronic circuitry and/or components, such as bond pads, filters, ADCs, digital-to-analog converters (DACs), AFEs, processors, memory, power management circuitry, communication interfaces, and/or any other suitable analog or digital circuitry. In some embodiments, the first electronic modules 918 include more complex integrated electronics, while the second electric modules 922 serve primarily to provide back side connections to other devices.

Referring next to FIG. 9B, the method can subsequently include bonding the subassemblies 900a-c to each other to create a single, larger assembly 930. The bonding process can include, for example, connecting a back side 906b of the first substrate 902 to the front side 914a of the second substrate 908, and connecting the back side 914b of the second substrate 908 to the first electronic modules 918 of the third substrate 916. The subassemblies 900a-c can be aligned with each other so that each microneedle array 904 can be electrically coupled to a corresponding first electronic module 918 by the first substrate 904 and a respective conductive interconnect 910.

Referring next to FIG. 9C, the method can include a first singulation step to cut partially or fully through the first substrate 902 to electrically isolate the microneedle arrays 904 from each other. The first singulation step can be performed by wet etching, dry etching, dicing, or any other suitable technique.

Referring next to FIG. 9D, the method can subsequently include a second singulation step to cut through the second and third substrate 908, 916 to create a plurality of individual sensor chips or packages 950 (“packages 950”). The second singulation step can be performed by wet etching, dry etching, dicing, or any other suitable technique. Each package 950 can include an individual microneedle array 904 connected to a respective first electronics module 918 via the first substrate 902 and the conductive interconnect 910. Each microneedle array 905 can also be connected to a respective second electronics module 922 via the first substrate 902, the conductive interconnect 910, first electronic module 918, and third substrate 916.

After singulation, the packages 950 can be further modified, functionalized, and/or otherwise processed as appropriate to prepare the package 950 for assembly into a larger biosensor device. Optionally, the packages 950 can be tested and/or calibrated using an electrochemical bath or test chamber, or other suitable mechanism. The electronics modules 918, 922 integrated into the packages 950 can be used during the downstream processing, testing, and/or calibration steps to provide various output data, allow for individualized tracking and/or processing, etc.

The method illustrated in FIGS. 9A-9D can be varied in many different ways. For example, although the illustrated embodiment depicts a process for manufacturing four packages 950, the method can be scaled up for any suitable number of packages 950 (e.g., tens, hundreds, or thousands of individual packages 950). Additionally, although the packages 950 shown in FIG. 9D each include a single microneedle array 904, in other embodiments, each package 950 can include multiple microneedle arrays 904 (e.g., two, three, four, five, ten, twenty, or more). In such embodiments, each microneedle array 904 within a single package 950 can be connected to a respective electronics module 918, multiple microneedle arrays 904 can be connected to the same electronics module 918, or any suitable combination thereof. As previously described, the number of conductive interconnects 910 in each package 950 can be varied as desired, e.g., depending on the number of microneedle arrays 904, the number of electronics modules 918, 922, the number of discrete conductive layers of each microneedle (e.g., as discussed above with reference to FIGS. 7A-7D), and/or any other suitable parameter.

In some embodiments, the manufacturing methods described herein allow electronic components to be integrated into the package 950 containing the microneedle array 904, which can provide some or all of the following advantages: reduced noise and/or increased sensitivity of the electronics due to the shorter signal path between the microneedle array 904 and the AFE; reduced noise can allow for the same or better sensitivity with fewer microneedles, which can increase the number of packages 950 that can be produced on a single wafer and/or lower cost; lowered costs and the integration of the electronics and microneedle array 904 into a single package 950 can allow for a fully disposable product; and/or integration of electronics and the AFE into each package 950 can allow most or all electrochemical depositions, functionalization, and inline tests to be performed using the integrated electronics, which can allow for massively parallelized single component tracking and factory calibration.

### IV. METHODS FOR BIOMONITORING AND HEALTHCARE GUIDANCE

In some embodiments, the present technology provide methods for monitoring a user's health state, predicting a future health state, and/or providing personalized healthcare guidance, based on measurements of health parameters (e.g., analyte levels, physiological values, etc.) generated by any of the systems and devices described herein. Any of the methods described herein can be performed by a system (e.g., the biomonitoring and guidance system 102 of FIG. 1), a user device (e.g., any of the user devices 104 of FIG. 1), a biosensor (e.g., the device 200 of FIG. 2, the device 300 of FIGS. 3A-3R), a cloud computing system, remote server, or any other suitable computing system or device, or a combination of any of the above.

For example, the methods herein can be used to generate a prediction of analyte levels at a future time period, such as a prediction for one or more of the following analytes: blood glucose (e.g., 30-day time-in-range and/or other time-in-range metric, a1c data), gases (e.g. oxygen, carbon dioxide, etc.), electrolytes (e.g., bicarbonate, potassium, sodium, magnesium, chloride, lactic acid), blood urea nitrogen (BUN), creatinine, ketones, cholesterol, triglycerides, alcohols, amino acids, neurotransmitters, hormones, disease biomarkers (e.g., cancer biomarkers, cardiovascular disease biomarkers), drugs, pH, cell count, and/or other biomarkers. Alternatively or in combination, the health data can be used to generate a prediction for physiological and/or behavioral parameters, such as weight, BMI, waist circumference, body fat percentage, heart rate, respiratory rate, body temperature, blood pressure, activity levels, sleep quality, stress levels, and/or combinations thereof. The prediction can be made for any suitable time period, such as 15 minutes, 30 minutes, 1 hour, 2 hours, 3 hours, 4 hours, 5 hours, 12 hours, 24 hours, 36 hours, 2 days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, or 12 months in the future from the date of the prediction.

The predictions can be used to provide recommendations, guidance, and/or other information for assisting a user in monitoring and/or managing a disease, condition, or other health state, such as any of the following: diabetes and associated conditions, liver diseases, cardiovascular diseases, cardiovascular health, cancer, lung diseases, renal diseases, renal health, brain conditions, ophthalmological diseases, intoxication, dehydration, hyponatremia, shock, heat stroke, infection, sepsis, trauma, water retention, bleeding, endocrine disorders, muscle breakdown, malnutrition, body function, gynecological diseases and conditions, pregnancy, fertility, drug use, physical performance, nutrition, mental and behavioral health, wellness, and/or combinations thereof. For example, the methods herein can be used to predict the progression of a disease or condition, and generate personalized guidance for actions that the user may take to improve, mitigate, and/or slow the progression of the disease or condition. As another example, the methods herein can be used to predict a user's future health state, and generate personalized guidance for actions to maintain and/or improve their health state. In yet another example, the methods herein can be used to predict whether a user will meet certain health goals, and generate personalized guidance for actions to increase the likelihood of meeting those health goals.

In some embodiments, the methods herein generate predictions based on current measurements of a user's health parameters generated by a biosensor, previous sensor data for that user, and/or sensor data from a plurality of other users. The predictions can be generated by one or more trained machine learning models. The model(s) can generate predictions of any of the following: a prediction of a future value of the health parameter in the near term, assuming the user does not significantly alter their behavior; a prediction of a future value of the health parameter in the near term, if the user takes one or more suggested actions; a prediction of the user's future health state in the long term, assuming the user does not significantly alter their behavior; and/or a prediction of the user's future health state in the long term, if the user alters their behavior and/or if there is some other therapeutic intervention. In some embodiments, the health state is quantified as a score or metric representing the user's overall health status and/or risk, which can be generated based on any suitable combination of sensor data and/or other data.

The predictions generated by the methods herein can be provided to the user to provide personalized healthcare guidance, e.g., in the form of suggested actions and/or interventions to improve the user's health. Alternatively or in combination, the predictions can be generated to other individuals who are responsible for or otherwise involved in caring for the users, such as home health aides, parents and/or other family members, healthcare providers, and the like. The predictions can also be used in healthcare settings, e.g., to inform physicians, nurses, paramedics, and/or other professionals of a patient's status for purposes of triage and/or diagnosis. This approach can be beneficial in situations where the patient may not have the capacity to communicate and needs proactive intervention from healthcare professionals (e.g., patients in an intensive care unit, surgical suite, or other critical care situations). Remote monitoring can also be advantageous for workers in high-risk environments (e.g., pilots, drivers, factory workers, etc.) where impending incapacitation, reduction in faculty, or other emergency situations could result in increased risk of damage or injury.

### A. Methods for Generating Health Predictions

In some embodiments, the present technology provides a computer-implemented method for forecasting one or more biological values or other health parameters. The method can include determining one or more features for training a biological forecasting model. The one or more features can be determined based on one or more input data parameters associated with a user in a plurality of users. The model can be trained based on the determined features, and the trained model can be used to generate one or more forecasted biological values. The model can identify correlations between determined features or other features to generate recommendations, forecasts, and other information.

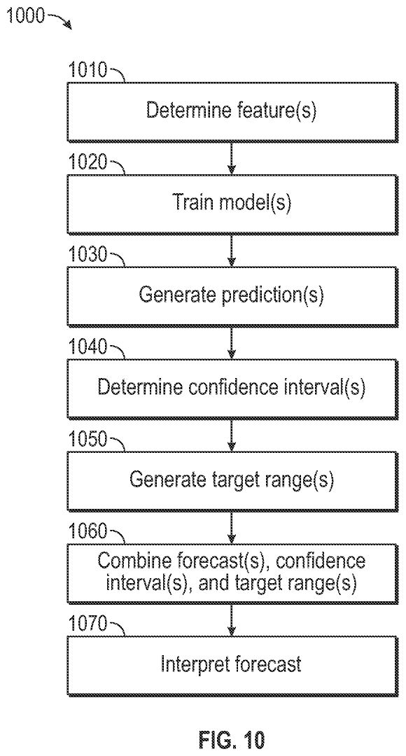

FIG. 10 is a block diagram illustrating a method 1000 for generating a prediction or forecast of a user's health parameters, in accordance with embodiments of the present technology. Although certain aspects of the method 1000 are described herein in connection with forecasting and interpretation of blood glucose concentration, it will be appreciated the method 1000 can be adapted for forecasting and/or interpreting levels of other types of analytes.

The method 1000 can begin at step 1010 with determining features for training a forecasting data model, also referred to herein as feature engineering. The features can be generated based on a multitude of factors and/or data, such as health measurements generated by a multi-analyte biosensor (e.g., the device 200 of FIG. 2, the device 300 of FIGS. 3A-3R), received from a user device (e.g., the user device 104 of FIG. 1), or any other suitable data source. For example, when forecasting blood glucose levels, the data can include data related to changes in blood glucose concentrations (e.g., whether in individuals with any type of diabetes, individuals without any type of diabetes, healthy with/out any other medical conditions, etc.) resulting from various activities (e.g., food intake, medication intake, physical activity, etc.), and/or data specifically related to metabolic processes that cause different factors (e.g., food, medication, etc.) to affect blood glucose values. The features calculated from the data can include, for example: averages over a specified time period, standard deviations over a specified time period, trends, fits (e.g., polynomial fits), timing-related features (e.g., duration of events, time elapsed between events), whether certain conditions are true or false (e.g., whether a particular event has occurred), and the like.

The candidate features can then be used in training and/or validation processes where a model can be trained with some of the candidate features using a first set of data (e.g., a training data set), then the accuracy of that model can be evaluated by using it to predict values from a second set of data (e.g., a validation data set). This process can be repeated with different sets of candidate features until the features that produce the best accuracy on the validation data are identified. These features can then be used in the subsequent steps of the method 1000 to train forecasting models and/or generate predictions. Because these processes can be repeated over time, specific features being used in the model can be changed and/or improved regularly.

At step 1020, the method 1000 can include training one or more forecasting models. As can be understood, any known machine learning models can be used for the purposes of training, testing, and/or validation, such as any of the machine learning models described above with respect to FIG. 1 (e.g., a gradient boosted model such as XGBoost, etc.). In some embodiments, the model is trained using data collected from all users up to and/or through a certain point in time, and/or using features generated from such data. The next period of collected data can be used for validation.

At step 1030, the method 1000 can include generating one or more predictions of health parameters for a particular user (e.g., predictions of analyte levels, such as blood glucose values). For example, the trained model can be used to generate predictions as follows. At the time the prediction is made, input data (e.g., user health data and/or features generated from user health data) can be obtained and/or generated for specific times (e.g., 30 minutes, 60 minutes, 90 minutes, etc.) into the future. The data can be generated for any suitable forecast period, such as a range from 8 hours to 12 hours in the future. Inputs (e.g., data and/or features generated from data) appropriate to each prediction time can be generated, and this set of inputs can then be provided to the forecasting model to generate predictions of health parameters. For example, when predicting blood glucose concentration, the inputs can include any of the following: the time of day (e.g., including month, day, and/or year), the mean of the user's past blood glucose values, the standard deviation of the user's past blood glucose values, the standard deviation of the changes in the user's past blood glucose values, the user's most recent blood glucose value, the time between the most recent blood glucose measurement and the prediction to be made, the most recent carbohydrates logged by the user, the time between the blood glucose prediction and the most recent logged carbohydrates, the estimated carbohydrate absorption between the previous blood glucose measurement and the prediction to be made, the user's most recent logged activity, the time between the most recent activity and the prediction to be made, an exponentially weighted moving average of the user's past logged activity, the most recent a1c value logged by the user, the time between the most recent a1c value and the prediction to be made, the most recent weight logged by the user, the time between the most recent weight and prediction to be made, the number of years the user has been diagnosed with diabetes, the time between when the user enrolled in the app and the prediction to be made, insulin type, and/or other diabetes medication. The inputs to be used can be varied as desired based on the particular health parameter prediction to be made.

At step 1040, the method 1000 can optionally include determining confidence intervals for the prediction(s). The process for determining confidence intervals can vary based on the type of training models used. In some embodiments, step 1040 includes determining one or more standard errors for the predicted values, and can also include a table of confidence intervals as a function of a standard error. At prediction time, the trained model can generate the forecast values and their standard errors, then determine one or more prediction confidence intervals depending on the standard errors determined by the confidence interval forecast.

At step 1050, the method 1000 can optionally include generating one or more target ranges for a health parameter. In some embodiments, a user can identify upper and/or lower limits that the user desires to stay between (e.g., from 70 mg/dL to 140 mg/dL, or from 70 mg/dL to 170 mg/dL for blood glucose values). This information can be used to help the user to interpret the forecast in terms of whether the forecast was in line with healthy values, above, below, etc. In embodiments where the target range may vary throughout the day (e.g., blood glucose target ranges may shift depending on when the user eats, performs various activities, etc.), multiple different target ranges can be generated for different time periods.

At step 1060, the method 1000 can optionally include combining forecast(s), confidence interval(s), and/or target range(s) for output to the user, e.g., via display on a user interface of a user device. The output can inform the user of likely near-term health parameter values and their uncertainties, can provide a useful reference for comparison, and/or can allow the user to make decisions about whether or not to change plans and/or take any action.

At step 1070, the method 1000 can optionally include interpreting the forecast(s). For example, the forecast values can be compared to the target range at the various forecast times. If more than a threshold percentage (e.g., 10% or 25%) of the forecast values are above the target range, the forecast can be labeled “high.” The system 100 can generate a message for display to the user (e.g., “likely to go higher than recommended within 4 hours,” “likely to remain within healthy levels for the next 8 hours”). The determination can also be used as an input to automatically select a support message that can provide the user with various actions that the user can undertake.

The method 1000 of FIG. 10 can be performed in many different ways. For example, any of the steps of the method 1000 can be omitted, repeated, combined with other steps, divided into additional sub-steps, etc. Additional examples and details of process steps suitable for use with the method 1000 are provided in U.S. Patent Application Publication No. 2020/0077931, which is incorporated herein by reference in its entirety.

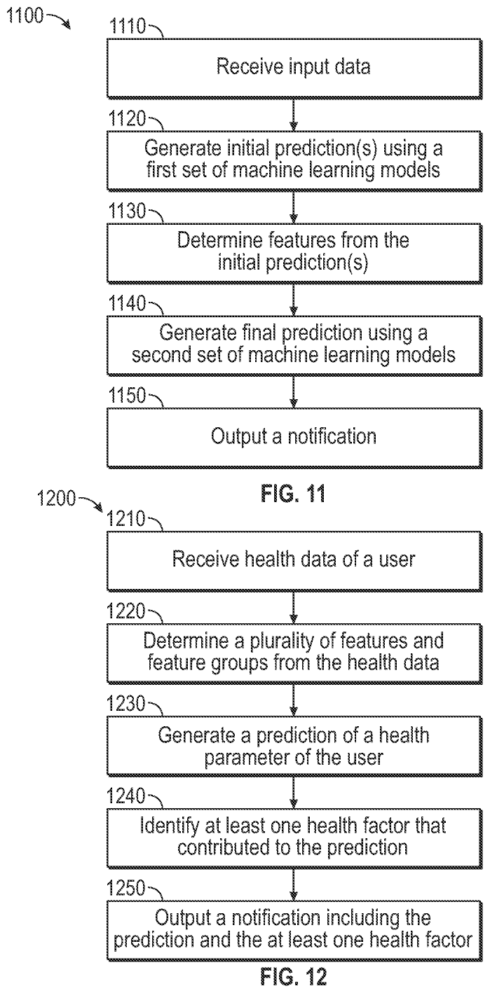

FIG. 11 is a block diagram illustrating a method 1100 for forecasting or predicting a health state of a user, in accordance with embodiments of the present technology. The health state can include, for example, a level or value for a health parameter (e.g., a blood glucose level or concentration), and/or the occurrence of a health-related event (e.g., an occurrence of a hypoglycemia event, and/or an occurrence of a hyperglycemia event). The prediction can be for a future time point (e.g., a health state at a time point that is 15 minutes, 30 minutes, 60 minutes, 90 minutes, 2 hours, or 4 hours into the future), or for a future time period (e.g., a health state over the next 15 minutes, 30 minutes, 60 minutes, 90 minutes, 2 hours, 4 hours, or overnight). For example, the method 1100 can be used to predict one or more health parameter values at one or more future time points, such as the forecasted value at a certain time interval (e.g., every 2 minutes, 5 minutes, 10 minutes, 15 minutes) over a specified time period (e.g., the next 30 minutes, 60 minutes, 90 minutes, 2 hours, 4 hours, or overnight). As another example, the method 1100 can be used to predict, for a particular future time period, whether the user's health parameter values are likely to fall below a particular threshold value (e.g., whether blood glucose levels are likely to fall below a threshold for hypoglycemia), whether the user's health parameter values are likely to rise above a particular threshold value (e.g., whether blood glucose levels are likely to rise above a threshold for hyperglycemia), whether a health-related event is likely to occur (e.g., in terms of low, medium, or high risk), and so on.

The method 1100 begins at step 1110 with receiving input data. The input data can include any suitable data described herein, such as health measurements generated by a multi-analyte biosensor (e.g., the device 200 of FIG. 2, the device 300 of FIGS. 3A-3R), received from a user device (e.g., the user device 104 of FIG. 1), or any other suitable data source. For example, when forecasting blood glucose levels, the data can include blood glucose data (e.g., continuous blood glucose data generated by a CGM device), insulin intake data, food intake data, physical activity data, etc. In some embodiments, the input data includes one or more “episodes” of substantially uninterrupted health measurements, which may be processed (e.g., smoothed) and/or correlated with at least event (e.g., medication intake, food intake, physical activity, etc.). Optionally, the input data can include only a single episode of health measurements (e.g., the most recent episode of the user), which can be annotated or otherwise correlated with one or more events. The data may be obtained from various sources, e.g., inputted by the user, queried from one or more databases, obtained from biosensor or other user devices, etc.

At step 1120, at least one initial prediction is generated using a first set of machine learning models. Specifically, the input data (e.g., an episode augmented with event data) is input into the first set of machine learning models, and the first set of machine learning models use the input data to generate the initial prediction(s). The first set of machine learning models can include any suitable number of machine learning models, such as one, two, three, four, or more different machine learning models. In embodiments where the first set includes multiple machine learning models, each model can independently generate a respective initial prediction of the user's health state. For example, depending on the number of machine learning models in the first set, step 1120 can include generating one, two, three, four, or more initial predictions. Optionally, some or all of the outputs of the machine learning models can be combined with each other to generate the initial prediction (e.g., using weighted averages, etc.).

The first set of machine learning models can include any suitable type of machine learning model, such as one or more of the machine learning models previously described with respect to FIG. 1. Each machine learning model can be trained on a respective set of training data. In embodiments where the first set of machine learning models includes multiple machine learning models, some or all of the models can be trained on the same training data, or some or all of the models can be trained on different training data. The training data can include, for example, previous data from the same user, as well as data from other users.

The initial prediction(s) generated by the first set of machine learning models can be a prediction of one or more future health parameter values (e.g., blood glucose levels), health events (e.g., a hypoglycemia event, a hyperglycemia event), or a combination thereof. For example, the initial prediction(s) can include a time series of health parameter values at a specified time interval over a specified time period (e.g., every 5 minutes for the next 1-2 hours). The initial prediction(s) can optionally include a calculated confidence interval or other indicator of uncertainty for each predicted health parameter value. In embodiments where the first set of machine learning models includes multiple different machine learning models, each model can produce a respective time series of analyte predictions. Optionally, the initial prediction(s) can be filtered, e.g., to exclude predictions that are outliers, inconsistent with the input data, and/or contradictory. Filtering can also be performed to exclude predictions that are more likely to be inaccurate (e.g., low confidence predictions) while retaining predictions that are more likely to be accurate (e.g., high confidence predictions).

At step 1130, one or more features are determined from the initial prediction(s). The features can include transformations, combinations, statistics, or any other properties or characteristics of the initial prediction(s). Features can include, but are not limited to: averages over a specified time period, standard deviations over a specified time period, trends, fits (e.g., polynomial fits), timing-related features (e.g., duration of events, time elapsed between events), whether certain conditions are true or false (e.g., whether a particular event has occurred), and the like. For example, in embodiments where the initial prediction includes a time series of predicted analyte levels, the features extracted from the prediction may include one or more of the following: average health parameter value, maximum health parameter value, minimum health parameter value, standard deviation of the health parameter value, an amount of time that the user's health parameter values are above or below certain thresholds, etc.

Optionally, step 1130 can also include generating features from other data, such as the input data from step 1110 (e.g., one or more augmented episodes). Features can also be generated from other data of the user such as personal data (e.g., age, gender, demographics, diabetes type), previous analyte data, meal data, medical history data, exercise data, personal data, medication data, physiological data, or any other data type described herein. Features may be generated from the data using transformations, combinations, statistics, and/or any other suitable technique for determining properties or characteristics of the user data.

At step 1140, at least one final prediction is generated using a second set of machine learning models. Specifically, the features determined at step 1130 are input into the second set of machine learning models, which generates the final prediction. In some embodiments, the features from step 1130 are the only input into the second set of machine learning models. In other embodiments, the second set of machine learning models can also receive other inputs, such as the input data of step 1110 (e.g., one or more augmented episodes), the initial prediction(s) generated in step 1120, and/or other user data (e.g., personal data, previous health parameter value data, meal data, medical history data, exercise data, personal data, medication data, physiological data, etc.).

The second set of machine learning models can be different from the first set of machine learning models. In some embodiments, the second set of machine learning models includes only a single machine learning model. In other embodiments, the second set of machine learning models can include multiple machine learning models whose outputs are combined (e.g., by weighted averages, etc.) to generate a single final prediction. The second set of machine learning models can include any suitable type of machine learning model, such as one or more of the machine learning models previously described with respect to FIG. 1. Each machine learning model can be trained on a respective set of training data. In embodiments where the second set of machine learning models includes multiple machine learning models, some or all of the models can be trained on the same training data, or some or all of the models can be trained on different training data. The training data can include, for example, previous data from the user and/or data from other users.

In some embodiments, the training data for the second set of machine learning models includes features generated from data of the user and/or data of a plurality of other users. The features can include any of the features previously described with respect to step 1130. In some embodiments, for example, the features can be generated from a plurality of user data sets, each user data set including personal data (e.g., diabetes type), analyte data (e.g., previous and/or current blood glucose data), medication intake data, food intake data, physical activity data, and/or any other data. Each user data set can also include health parameter value predictions for the user that are generated using machine learning models (e.g., the first set of machine learning models). The health parameter value predictions can be retrospective predictions generated from previous health parameter value data. The features generated from these predictions can also be used to train the second set of machine learning models.

The final prediction produced by the second set of machine learning models can be a prediction of one or more future health parameter values (e.g., blood glucose levels), a health-related event (e.g., a hypoglycemia event, a hyperglycemia event), or a combination thereof. For example, the final prediction can be a predicted series of health parameter values over a specified time period and at a specified time interval (e.g., every 5 minutes for the next 1-2 hours). As another example, the final prediction can be an estimated likelihood that the user will experience a health-related event within a specified time period (e.g., the next 15 minutes, 30 minutes, 60 minutes, 90 minutes, 2 hours, 4 hours, or overnight). The likelihood of the health-related event can be expressed in various ways, such as in qualitative terms (e.g., “likely to occur” versus “not likely to occur,” “high risk” versus “moderate risk” versus “low risk”) and/or in quantitative terms (e.g., a probability value). Optionally, the final prediction can be filtered, e.g., to exclude predicted values that are outliers, inconsistent with the input data, and/or contradictory (e.g., as previously described with respect to step 1120).

At step 1150, the method 1100 optionally includes outputting a notification to the user. The notification can be output by the system for display on a user device (e.g., user devices 104 of FIG. 1) via a graphical user interface, as described in greater detail below. The notification can include information regarding the final prediction of the health state (e.g., the predicted health parameter value, the predicted likelihood of a health-related event, etc.). In some embodiments, the notification includes recommendations or feedback on actions that the user may take in response to the predicted health state, e.g., to increase, decrease, or maintain the health parameter values; to avoid the occurrence of a health-related event; to increase the likelihood of the health-related event occurring; etc. For example, the notification may instruct the user to take medication, consume a meal, exercise, contact a healthcare professional, and so on. Optionally, the user can be transmitted to a physician or other healthcare professional associated with the user, e.g., if the final prediction indicates that the user may need immediate medical attention or if there are any other situations where the physician should be notified.

The method 1100 of FIG. 11 can be performed in many different ways. For example, any of the steps of the method 1100 can be omitted, repeated, combined with other steps, divided into additional sub-steps, etc. Additional examples and details of process steps suitable for use with the method 1100 are provided in U.S. Patent Application Publication No. 2020/0375549, which is incorporated herein by reference in its entirety.

FIG. 12 is a block diagram illustrating a method 1200 for forecasting or predicting a health parameter of a user, in accordance with embodiments of the present technology. The method 1200 begins at step 1210, the method 1200 begins with receiving health data of a user. The health data can include any data relevant to the user's health state, such as any of the following: blood pressure data (e.g., current and/or previous measurements of systolic and/or diastolic blood pressure), blood glucose data (e.g., current and/or previous blood glucose measurements, current and/or previous HbA1c data values), heart rate data, food data (e.g., number of meals; timing of meals; number of calories; amount of carbohydrates, fats, sugars, etc.), medical history data (e.g., current and/or previous weight, height, BMI, age, sleeping patterns, medical conditions, cholesterol levels, diabetes type, family history, user health history, diagnoses, blood pressure, etc.), activity data (e.g., time and/or duration of activity; activity type such as walking, running, swimming; strenuousness of the activity such as low, moderate, high; etc.), personal data (e.g., name, gender, demographics, social network information, etc.), medication data (e.g., timing and/or dosages of medications such as insulin, prescription and/or non-prescription medications taken), and/or any other suitable data (e.g., basal energy consumption, oxygen consumption) or combination thereof. The health data can include health measurements generated by a multi-analyte biosensor (e.g., the device 200 of FIG. 2, the device 300 of FIGS. 3A-3R), received from a user device (e.g., the user device 104 of FIG. 1), or any other suitable data source.

Optionally, step 1210 can further include receiving a health goal for the user. The health goal can be a target value and/or range for a particular health parameter (e.g., blood pressure, blood glucose, weight, etc.) that the user wishes to achieve in the future (e.g., one, two, three, four, five, six, or more months in the future). For example, the health goal can be for the user's health parameter to achieve a target value and/or range, to be greater than a target value and/or range, to be less than a target value and/or range, etc. The health goal can be determined by the user, by a healthcare professional, set based on healthcare guidelines (e.g., based on the user's characteristics), or suitable combinations thereof. The health goal can be input by the user via a user device, transmitted to the system from a healthcare professional's device, retrieved from a database or server, or any other suitable technique. Optionally, step 1210 can include receiving multiple health goals for multiple different health parameters.

At step 1220, the method 1200 continues with determining a plurality of features and feature groups from the health data of step 1210. The features can be determined from the health data using any suitable approach. For example, the features can include values (e.g., the most recent value or set of values) and/or statistics (e.g., averages, standard deviations, ranges, sums, differences, ratios, maximums, minimums, percentiles, probabilities, cross-correlations, time-in-range values) for any suitable health data, including, but not limited to, any of the following: analyte levels (e.g., blood glucose levels), physiological parameter values (e.g., blood pressure levels, heart rate), weight, food intake, medical history, demographics, diagnoses and/or medical conditions, medications, sleep patterns, activity patterns, and/or combinations thereof. Features can be computed across health data obtained over any suitable length of time (e.g., 15 days, 30 days, 60 days, or 90 days) and at any suitable time period before the prediction is made (e.g., immediately before the prediction, 15 days before the prediction, 30 days before the prediction, 60 days before the prediction, or 90 days before the prediction).

In some embodiments, the features are classified in a plurality of feature groups, each feature group being associated with a respective health parameter. Each health factor can relate to an aspect of the user's health that may influence the predicted health parameter. Examples of health factors that may be used to determine feature groups include, but are not limited to: blood pressure, blood glucose, heart rate, multi-factor interactions, demographic factors, meal intake, sleep, weight, activity, medical conditions and/or diagnoses, and the like. Each feature in a particular feature group may be derived from measurements and/or other data for the corresponding health factor. In some embodiments, the features can be categorized into at least one, two, three, four, five, ten, or more different feature groups.

At step 1230, the method 1200 can include generating a prediction of a health parameter of the user. The prediction can be generated by inputting at least some of the features determined in step 1220, and, optionally, at least some of the health data received in step 1210, into the prediction model(s). In some embodiments, the prediction model(s) are or include one or more machine learning models (e.g., a Gradient Boosted Trees model). In such embodiments, the machine learning model(s) can be trained on health data from a plurality of different users. The training data may include data for the particular user for which the prediction is to be generated, or may not any include any data from the particular user. The use of training data from a large number of users allows accurate predictions to be made even for users with limited, irregular, and/or incomplete health data.

The prediction can provide an estimated value and/or range for the health parameter at a future time point. The future time point can be at least one, two, three, four, five, six, seven, eight, nine, ten, 11, 12, or more months from the date of the prediction. Alternatively or in combination, the prediction can provide an estimated probability that the health parameter will achieve a particular target value and/or range at the future time point. In such embodiments, the predicted probability can be expressed quantitatively (e.g., an x % chance of achieving the goal) and/or qualitatively (e.g., highly likely, likely, unlikely, highly unlikely).

At step 1240, the method 1200 can also include identifying at least one health factor that contributed to the prediction. The health factor can be identified in various ways, such as by selecting one or more feature groups that provided a threshold contribution to the prediction, then determining the health factor(s) associated with the selected feature group(s). For example, the contribution of at least some of the features used in steps 1220 and 1230 can be determined using an attribution algorithm (e.g., a SHAP algorithm) or other suitable technique. The attribution algorithm can be configured to calculate a quantitative value (e.g., a marginal contribution value) representing the contribution of each feature to the prediction of the health parameter. Subsequently, the contributions of each feature within a feature group can be aggregated (e.g., summed) to generate a subtotal representing the net marginal contribution of that particular feature group. The magnitude of the contribution of each feature group can correlate to the influence of that feature group on the final prediction, e.g., a larger magnitude can indicate a more influential feature group, a smaller magnitude can indicate a less influential feature group, etc.

Based on the determined contributions, step 1240 can further include identifying one or more feature groups determined to have contributed to the prediction (e.g., feature group(s) whose contribution met a threshold value and/or other suitable criteria). For example, step 1240 can include identifying at least one, two, three, or more feature groups having the greatest contribution(s) to the prediction, e.g., by ranking the feature groups in order of contribution magnitude. As another example, feature groups can be identified based on the percentage and/or proportion of the contribution made to the prediction, e.g., all feature groups contributing at least 10%, 25%, 50%, or 75% to the prediction; feature groups that collectively account for at least 50%, 75%, 90%, or 95% of the prediction; and so on.

At step 1250, the method 1200 can include outputting a notification to the user. The notification can include the prediction of the health parameter and, optionally, the at least one health factor determined to have contributed to the prediction (e.g., as discussed above with reference to step 1240). For example, if the blood glucose feature group was determined to have contributed to the prediction, the notification can include a support message or other feedback informing the user that the predicted outcome can be at least partially attributed to the user's blood glucose levels. The notification can be provided in any suitable format, such as textual, visual, graphical, audible, and/or other formats. The notification can be output to the user via a graphical user interface on a user device or any other suitable computing device.

The method 1200 can optionally include determining a recommended action for the user to improve their health parameters, based on the prediction and/or contributing health factor(s). For example, if the method 1200 determines that a certain health factor is particularly influential in causing the user to achieve or not achieve their health goal, the notification can inform the user with recommended actions with respect to that health factor (e.g., decreasing blood glucose levels; increasing physical activity; improving sleep patterns; altering dietary intake; etc.). The recommendation can be customized to the particular user, e.g., based on user feedback, behavioral patterns, etc. For example, the method 1200 can account for whether the user has historically complied or not complied with a particular lifestyle change, whether the user has expressed a preference for certain types of behavioral interventions, etc. Such information can also be used as input for generating future recommendations and/or other notifications to assist the user in meeting their health goals.

The method 1200 of FIG. 12 can be performed in many different ways. For example, any of the steps of the method 1200 can be omitted, repeated, combined with other steps, divided into additional sub-steps, etc. Additional examples and details of process steps suitable for use with the method 1200 are provided in U.S. patent application Ser. No. 17/167,795, which is incorporated herein by reference in its entirety.

### B. Methods for Using Predictions to Enhance Sensor Performance

In some embodiments, the methods herein use predictions of health parameter values (e.g., blood glucose levels) to fill in missing and/or erroneous sensor data, e.g., due to equilibration, anomalies, sensor dropouts, etc. For example, when a sensor is being changed out (e.g., a first disposable patch is being replaced with a second disposable patch), there may be a period of missing and/or erroneous sensor data while the new sensor is equilibrating. The equilibration period may range from 1 hour to 24 hours, depending on the sensor type, user physiology, etc., which may create a gap in health monitoring.

The forecasting methods described herein (e.g., in connection with FIGS. 10-12) can be used to predict health parameter values over time during the equilibration period, without relying on data from the new sensor. In some embodiments, for example, the method can include identifying a time period of missing and/or erroneous sensor data, e.g., based on sudden loss of or changes in the sensor signal, user input indicating that the sensor is being changed out, machine learning-based analysis of the sensor signal, etc. The method can then include generating a prediction of one or more health parameter values for the identified time period, in accordance with any of the forecasting methods described herein. Subsequently, the method can include replacing the missing and/or erroneous sensor data with the prediction to generate a continuous or substantially continuous data stream. Accordingly, the method can allow monitoring to continue, even during sensor changes and/or the equilibration period. Alternatively or in combination, the method can include use machine learning models and/or other user data to predict the amount of sensor drift during equilibration, and can use the predicted drift to modify the data generated by the new sensor during the equilibration time period to improve accuracy. The sensor can also use predictions to determine equilibration periods, identify erroneous sensor data, and/or develop calibration routines.

In some embodiments, the methods herein can be used to detect and/or compensate for sensor anomalies. Sensor anomalies may occur, for example, due to user activity (e.g., sleeping, exercise), environmental conditions (e.g., extreme cold or heat, altitude changes, water exposure), and/or other contextual factors. Anomaly detection can be performed in many different ways. For example, a method for detecting sensor anomalies can include comparing sensor data obtained during a particular time period to a prediction of the sensor data for that time period. The prediction can be generated using any of the methods described herein (e.g., in connection with FIGS. 10-12). If the actual sensor data differs significantly from the predicted sensor data (e.g., the health parameter value generated from the actual sensor data is significantly higher or lower than the prediction for that value), the method can determine that a sensor anomaly has occurred. Alternatively or in combination, the method can detect sensor anomalies using data from other sensors and/or devices, such as motion sensors (e.g., accelerometers, gyroscopes), heart rate sensors, temperature sensors, location sensors, pressure sensors, optical sensors, etc. In some embodiments, the method includes obtaining data from a plurality of sensors and/or devices to assess the user's current state, surrounding environment, and/or other contextual information, and using the contextual information to detect the likelihood of sensor anomalies. In some embodiments, the method uses trained machine learning models to identify instances of sensor anomalies. If the method detects that a sensor anomaly is occurring or is likely to occur, the method can modify and/or exclude anomalous sensor data, e.g., by alter the operating parameters (e.g., filtering parameters), omitting sensor data from certain time period, etc. Optionally the method can modify or replace the anomalous sensor data with the corresponding predicted sensor data.

For example, the methods described herein can be used to detect and/or compensate for reduction and/or loss of sensor signal due to pressure, also known as “pressure-induced dropout.” For sensors that are configured to sample the user's interstitial fluid (e.g., CGM sensors), when the sensor and/or surrounding tissues are compressed (e.g., the user rolls onto the sensor during sleep or inadvertently presses against the sensor), the pressure may displace the interstitial fluid near the sensor, causing a reduction or loss of signal. The dropout period may range from a few minutes to several hours, depending on the user's activity patterns. The dropout may interfere with the accuracy of the monitoring and/or prediction processes. For example, in the context of glucose sensing, sensor dropout may be erroneously interpreted as a hypoglycemia event.

Accordingly, the methods herein can be used to detect when sensor dropout is occurring or is likely to occur. For example, a method can include identifying a time period in which sensor dropout is likely to occur or is occurring. The identification can be based on data from other sensors, such as by using an activity tracker to determine when the user is sleeping or otherwise stationary, one or more pressure sensors at or near the sensor at issue, etc. The method can then include detecting whether a loss or significant reduction in sensor signal has occurred during this time period. In some embodiments, for example, the method uses models to analyze the sensor data to identify and/or predict instances of sensor dropout. The models can be trained using pressure-induced drop out data. If a sensor dropout event is detected, the method can include generating predictions of sensor data for the dropout period (e.g., using the techniques described in connection with FIGS. 10-12). The method can then use the predictions to replace and/or modify the sensor signal during the dropout period, thus allowing monitoring to continue in an uninterrupted or substantially uninterrupted manner. This approach can also reduce the incidence erroneous user alerts, such as false hypoglycemia alerts due to pressure-induced sensor dropout.

In some embodiments, the methods herein can be used to compensate for sensor lag. The sensor data for a particular health parameter may lag the actual biological values for that parameter due to physiological dynamics (e.g., analyte levels in the interstitial fluid may not immediately analyte levels in the blood). Delays may also be introduced during signal processing and/or analysis. For example, algorithms for filtering noisy sensor data can introduce time delays, in that the filtered signal may be time-shifted relative to the original signal. Sensor lag can interfere with monitoring and/or prediction accuracy. Additionally, in embodiments where the sensor data is used as feedback to control drug delivery (e.g., for closed loop insulin pumps and/or other delivery devices), sensor lag can cause instability, overcorrections, and/or oscillations. To address these issues, the methods herein can generate predictions of sensor data, e.g., using the techniques described in connection with FIGS. 10-12. The prediction can then be time-shifted by a determined time value to reduce or eliminate any sensor lag. The time value can be a fixed value (e.g., based on knowledge or estimates of the amount of sensor lag), or can be a variable value (e.g., calculated based on contextual information, determined using machine learning models or other techniques, etc.). This approach can provide instantaneous or near instantaneous predictions of health parameters that more accurately represent the actual dynamics of the user's physiology.

### V. USER INTERFACES

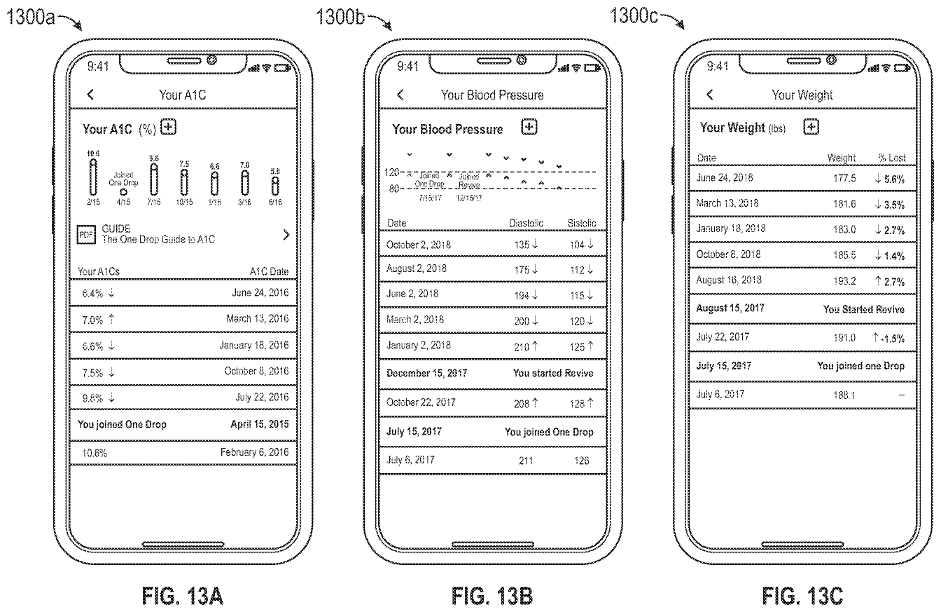

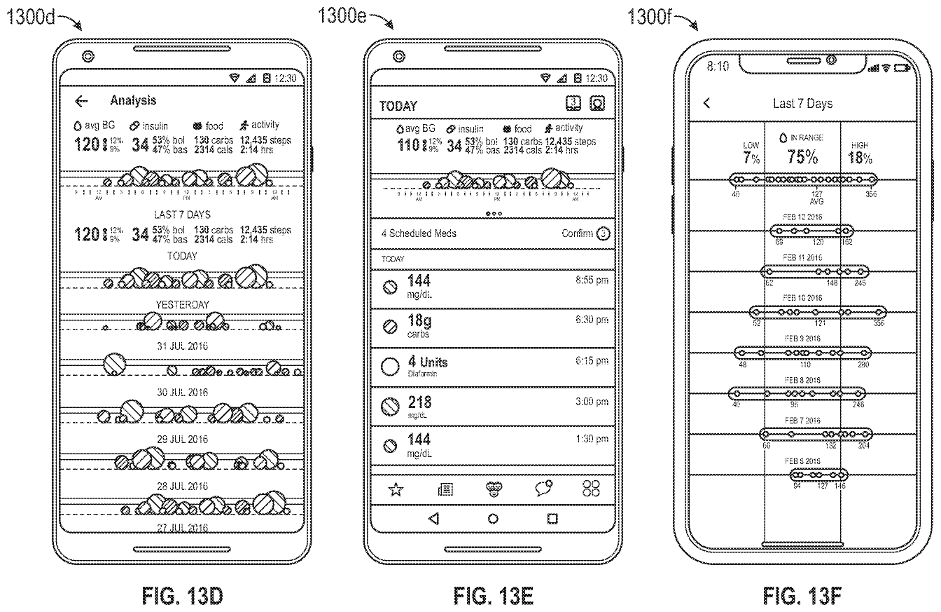

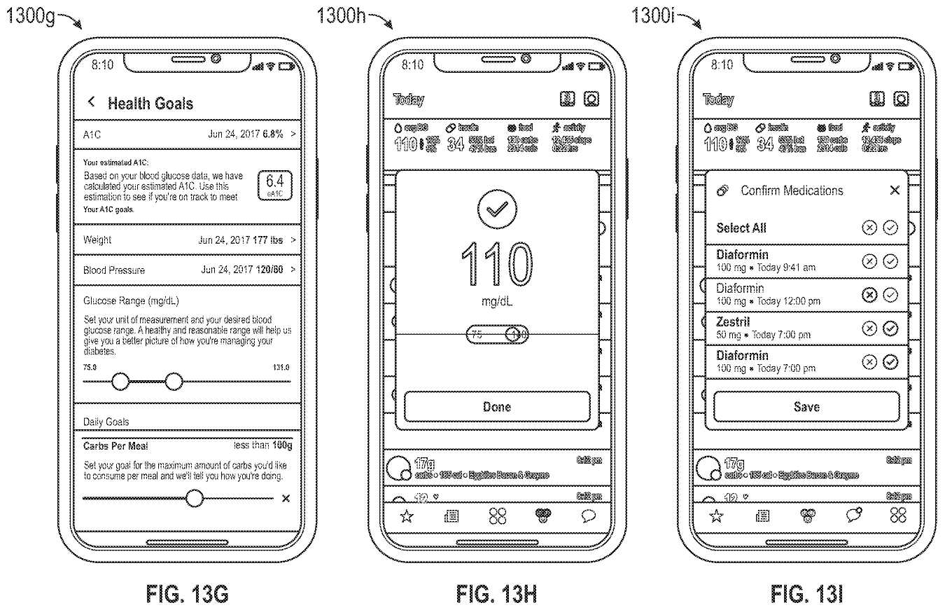

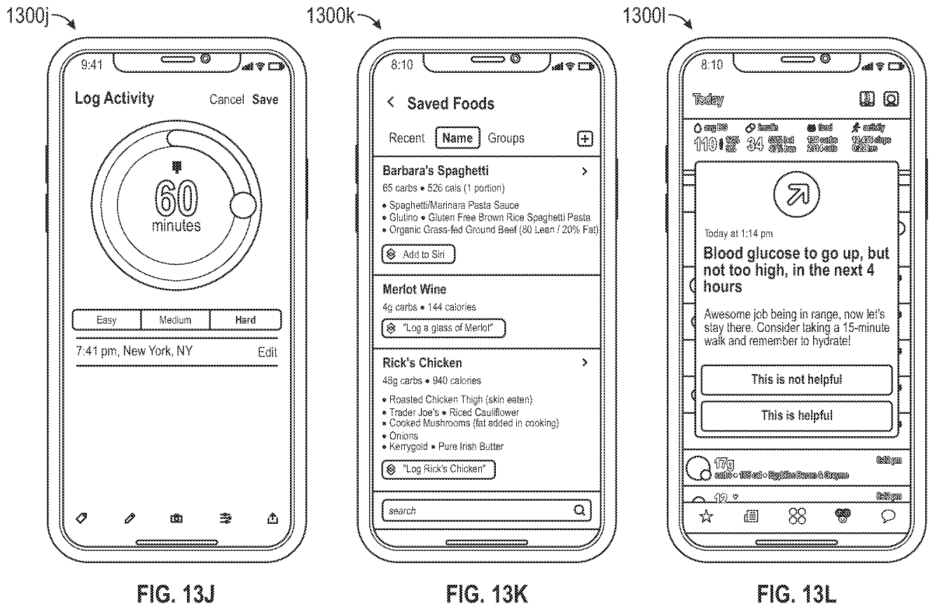

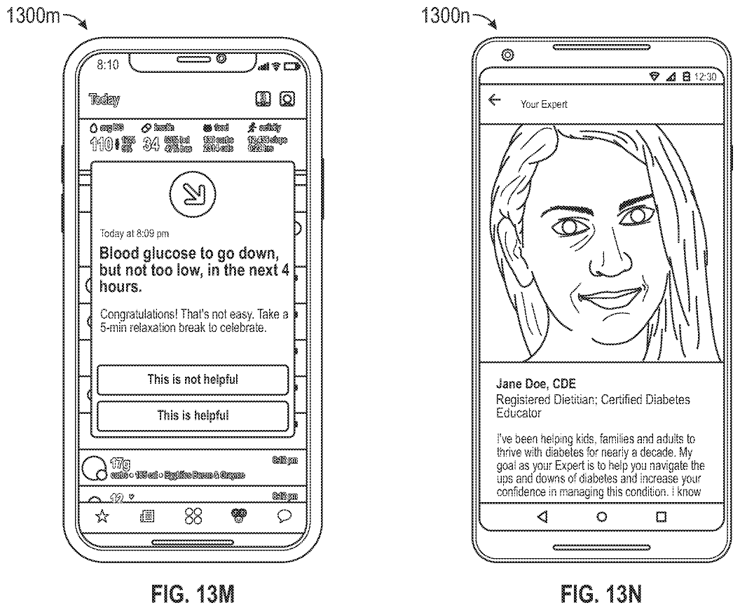

FIGS. 13A-13N illustrate various examples of user interfaces 1300a-n configured in accordance with embodiments of the present technology. The user interfaces 1300a-n can be displayed on any of the user devices described herein (e.g., the user device 104 of FIG. 1), such as a mobile device, smartwatch, laptop computer, personal computer, etc. The information presented on the user interfaces 1300a-n can be generated using any embodiment of the systems, devices, and methods described herein. Any of the features of the user interfaces 1300a-c can be combined with each other. Additionally, although the information shown in FIGS. 13A-13N relates to blood glucose monitoring, this is merely for illustrative purposes, and in other embodiments the user interfaces 1300a-n can display information for other types of health parameters (e.g., monitoring levels of other analytes and/or other physiological data).

FIG. 13A illustrates a user interface 1300a for tracking a user's health parameter values. In the illustrated embodiment, the user interface 1300a includes a timeline or graph showing a series of a1c values over time, as well as a chronological feed showing trends in recent a1c values (e.g., percent change relative to the previous values). Optionally, the user interface 1300a can also include a link to resources providing information regarding a1c measurements.

FIG. 13B illustrates another user interface 1300b for tracking a user's health parameters. The user interface 1300b can be generally similar to the user interface 1300a of FIG. 13A, except that the user interface 1300b displays the user's diastolic and systolic blood pressure values over time. The user interface 1300b can include a graph showing a series of blood pressure measurements overlaid onto a target blood pressure range (e.g., 80 mmHg to 120 mmHg).

FIG. 13C illustrates yet another user interface 1300c for tracking a user's health parameters. The user interface 1300c can be generally similar to the user interface 1300a of FIG. 13A, except that the user interface 1300c displays the user's weight over time, as well as the change in weight (e.g., percentage of weight lost) relative to previous weight measurements.

FIG. 13D illustrates a user interface 1300d for displaying health event data. The user interface 1300d can show a plurality of timelines for multiple days (e.g., the current day and/or the past few days). Each timeline can be annotated with visual indicators (e.g., icons) representing events that occurred during that day. In the illustrated embodiment, for example, the events include blood glucose measurements, insulin intake, food intake, and activity. The size of the visual indicators can also provide information regarding the event, e.g., larger icons can mean a higher blood glucose level, a higher insulin dose, more food intake, more activity, etc.

FIG. 13E illustrates another user interface 1300e for displaying health event data. The information shown on the user interface 1300e can be similar to the information shown on the user interface 1300d of FIG. 13D, except that the user interface 1300e displays health events for a single day. In the illustrated embodiment, the user interface 1300e displays an annotated timeline with the health events that occurred during the day, as well as a chronological feed providing more details on each event (e.g., blood glucose concentration, carbohydrates consumed, amount of insulin taken, duration of activity, etc.).

FIG. 13F illustrates a user interface 1300f for displaying time-in-range data. The user interface 1300f can show a plurality of timelines for multiple days (e.g., the current day and/or the past few days). Each timeline can be annotated with visual indicators (e.g., icons) representing measured values for a health parameter during that day. The timelines can also include graphics (e.g., a highlighted bar) showing the total range of values for that day. The user interface 1300f can also display the targeted range for that health parameter, e.g., as a highlighted region or other graphic overlaid onto the individual timelines, so the user can visualize how their actual health parameter values compare to the target range. The user interface 1300f can also display the amount of time the measured values were in the targeted range, below the targeted range, and/or above the targeted range.

FIG. 13G illustrates a user interface 1300g for inputting health goal data. The interface 1300g can allow the user to indicate target values and/or ranges for one or more health parameters, such as weight, blood pressure, blood glucose levels, a1c values, carbohydrate intake, etc. The interface 1300g can optionally display current values for the health parameters (e.g., the current average over a particular time period, the last measured value, etc.) so the user can assess their progress in achieving their health goals.

FIG. 13H illustrates a user interface 1300h for inputting health parameter measurements. The interface 1300h can allow the user to manually enter a value for a health parameter (e.g., blood glucose concentration). The interface 1300h can be used in situations where the health parameter is measured using a separate sensor that is not in communication with the user device (e.g., a blood glucose meter).

FIG. 13I illustrates a user interface 1300i for inputting medication data. The user interface 1300i can allow the user to input the types and amounts of medication they are currently taking or have taken, as well as the time each medication was taken. In some embodiments, the user interface 1300i can display a dialog box showing a predetermined medication schedule (e.g., based on previous information input by the user, information received from a healthcare provider, etc.), and the user can simply confirm whether the medication was actually taken according to the schedule.

FIG. 13J illustrates a user interface 1300j for inputting activity data. In the illustrated embodiment, the interface 1300j allows the user to specify the time spent on the activity, the strenuousness of the activity (e.g., “easy,” “medium,” “hard,”), the date and time of the activity, and/or the location where the activity was performed.

FIG. 13K illustrates a user interface 1300k for inputting meal data. The user interface 1300k can allow the user to input and save recipes for particular foods. Alternatively or in combination, the user interface 1300k can allow the user to search for and retrieve food data from databases and/or other data sources. The user interface 1300k can also display nutritional information so the user can evaluate the health impact of consuming that food. The user can select one or more foods that they have consumed or will consume in order to track their dietary intake over time.

FIG. 13L illustrates a user interface 13001 for displaying a health parameter prediction. In the illustrated embodiment, the user interface 13001 includes a dialog box with a message informing the user of the prediction (e.g., “Blood glucose to go up, but not too high”) and the time frame for the prediction (e.g., “next 4 hours”). The message can optionally include supportive statements (e.g., “Awesome job being in range”) and/or recommended actions (“Consider taking a 15-minute walk and remember to hydrate!”). The user interface 13001 also allows the user to provide feedback on the prediction and other displayed information (e.g., “This is not helpful,” “This is helpful.”)

FIG. 13M illustrates another user interface 1300m for displaying a health parameter prediction. The user interface 1300m is generally similar to the user interface 13001 of FIG. 13L, except that the content of the message has been adjusted to reflect the most recent prediction (e.g., “Blood glucose to go down, but not too low”).

FIG. 13N illustrates a user interface 1300n for displaying healthcare provider information. The user interface 1300n can show information for the user's current healthcare provider, or can show information for healthcare providers that the user may contact for diagnoses, guidance, prescriptions, etc.

### VI. ADDITIONAL EMBODIMENTS

In some embodiments, the systems described herein are configured to analyze user data and to generate personalized healthcare information for one or more conditions (e.g., chronic conditions, acute conditions, etc.), diseases, or the like. The healthcare information can be used to, for example, manage chronic conditions, monitor conditions or diseases, predict or identify acute conditions, and/or improve overall health, and can include sensor data (e.g., raw data, filtered data, calibrated data, etc.), recommendations, reports, forecasting, other health-related information, contextual information, or other relevant information usable to support, for example, telehealth and/or self-management. A user can access the healthcare information using mobile devices (e.g., a smartwatch, smartphone, tablet, etc.), computers, or other computing devices configured to output or display information. The user can input information (e.g., medical information, goals, dietary information, alert criteria, security settings, contact information, contextual information, etc.), control access to the personalized healthcare information, and manage usage of the personalized healthcare information. Healthcare information, contextual information, and/or other relevant information can also be automatically received from and/or reported to one or more linked data sources, such as databases, mobile devices, wearable devices, sensors, etc.

The healthcare information can include, for example, current real-time data, historical data, patterns, vital sign data, medication data, activity data, meal data, molecular and imaging diagnostic data, and/or automated decision support. Multiple sets of personalized healthcare information can be provided to manage multiple conditions, achieve multiple goals, or the like. For example, for diabetic patients, the historical data can include historical glucose data; the patterns can include blood sugar patterns; the medication data can include medication schedules, medication dosages, and/or insulin pump basal rates; and the automated decision support can predict blood sugar levels and provide one or more recommendations (e.g., food recommendations, activity recommendations, etc.) to treat diabetes mellitus. As another example, for overweight patients, the historical data can include historical weight data, the patterns can include eating patterns, and the automated decision support can provide an exercise program (e.g., exercise routines, schedules, goals, etc.), an eating program, etc. The user data can be monitored to detect heart attacks or other emergency conditions.

Healthcare information may be received from a CGM biosensor device including a chemical glucose sensor and an electrochemical glucose sensor configured to operate concurrently or sequentially. The healthcare information can include sensor data (e.g., raw data, filtered data, etc.) from one or both sensors. In some embodiments, the data collected by the CGM biosensor device can be locally and/or remotely analyzed. The analysis of the user's body chemistry can be provided to the user and/or one or more entities (e.g., health care professionals, physician, caretakers, relatives, friends, acquaintances, etc.). In some embodiments, a user's body chemistry is provided upon the user's request, sporadically, and/or periodically. The number, configurations, and functionality of the sensors in a biosensor device can be selected based on desired sensing capabilities.

The system can receive data from the user, sensors, biosensor devices, databases, medical devices, and other sources. A user can input their medical history, vitals, targets or goals, preferences, or the like. The sensors can be invasive, minimally invasive, or non-invasive. In some embodiments, the system can periodically or continuously receive data from a remote database. The user can link a user account of the system with a third-party account for automatic transfer of data. The data from the third-party account can include diagnostic data, health records, or the like such that the system can aggregate the data together to provide comprehensive analytics. The data from medical devices can include, for example, operational information (e.g., dosages, drug delivering schedules, etc.), diagnostic data (e.g., vitals, metabolic data, etc.), or the like.

The system can include one or more machine learning models trained based on user data (e.g., a user's data, a group of users, etc.). In certain embodiments, an analysis module can be configured with one or more algorithms to generate personalized information using statistics, machine learning, AI, neural networks, or the like. In some embodiments, one or more algorithms are used to identify correlations between data sets (e.g., data sets for the user, data sets from different users, data sets for populations), user parameters, healthcare provider parameters, and/or treatment outcomes. The data sets can include, without limitation, medical device-specific datasets, user-specific datasets, aggregated datasets, datasets generated using simulations, or the like. One or more correlations can be used to develop at least one predictive model that generates forecasts, certainty scores for forecasts, and other healthcare information.

The system can include a software module or engine that includes or communicates with an interface that accepts inputs from the user (e.g., user health condition, user characteristics, user activity), and uses these inputs to provide an output. The software module can also include an interface that renders an analysis based on sensed analytes and/or user inputs in some form. In an example, the software module includes an interface that summarizes analyte parameter values in some manner (e.g., raw values, ranges, categories, changes), provides a trend (e.g., graph) in at least one analyte parameter or body chemistry metric, provides alerts or notifications, provides additional health metrics, and provides recommendations to modify or improve body chemistry and health metrics. In another example, the software module can implement two interfaces: a first interface accessible by a user, and a second interface accessible by a health care professional servicing the user. The second interface can provide summarized and detailed information for each user that the health care professional interacts with, and can further include a message client to facilitate interactions between multiple users and the health care professional. The software module can additionally or alternatively access a remote network or database containing health information of the user. The remote network can be a server associated with a hospital or a network of hospitals, a server associated with a health insurance agency or network of health insurance agencies, a server associated with a third party that manages health records, or any other user- or health-related server or entity. The software module can additionally or alternatively be configured to accept inputs from another entity, such as a healthcare professional, related to the user.

Inputs may be received from wearable sensors. In one implementation, data available from a smart watch may be used either on a standalone basis or to augment other data. In some examples, sensors and signals collected by smart watches (e.g., Apple watch, Microsoft Band, etc.) or other wearable and/or mobile devices may be employed to augment detection of hypoglycemia. Such signals can include those from heart rate sensors, sympathetic/parasympathetic balance (which can be inferred from heart rate), perspiration/emotion/stress from conductance sensors, and motion data from accelerometers. Such signals may be used in addition to the continuous glucose monitoring (CGM) signals. The algorithms used to process these auxiliary signals can be trained on the user's own data, using CGM to assist in the training. These algorithms can be optimized via a system or device remote from the user's device, e.g., in the cloud. Then detection criteria can be sent to the user's smart phone, smart watch, and/or other wearable and/or mobile devices. There may be instances when CGM fails to detect hypoglycemia, but when augmented with auxiliary signals indicating possible hypoglycemia, the user may be alerted to the suspected hypoglycemia and thereby enabled to avoid the consequences. Alternatively, after the algorithms used to process the auxiliary signals have been trained, the smart watch signals may be able to detect hypoglycemia without the use of CGM. In this use case, adjustments to the algorithms may be necessary or desired to optimize sensitivity or specificity. Wearable sensors can output heart rates, blood pressure, skin temperatures, and other data.

The systems can manage sensors (e.g., calibration routines, testing settings, triggers, user controllable settings, etc.), drug delivery, and mobile apps. In some embodiments, the system can integrate with other systems or devices, such as virtual assistants (e.g., Alexa) and wearables (e.g., adaptive algorithms or analysis based on data from other devices, such as connected watches). User settings and physician controls/settings can be set via a wide area network, a local area network, or direct user input. The systems provide management of user data, alerts (e.g., user alerts, family members alerts, physician alerts, etc.), notifications, reports, encryption, and pairing with endpoint devices.

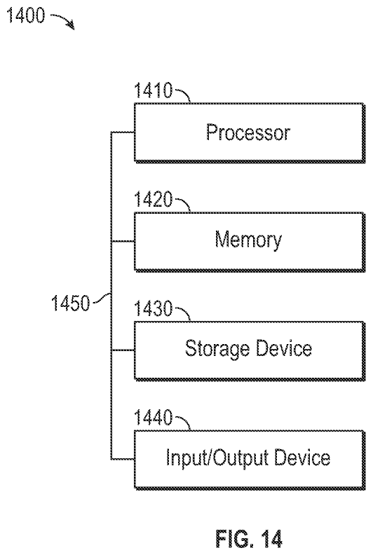

FIG. 14 is a schematic block diagram of a computing system or device (“system 1400”) configured in accordance with embodiments of the present technology. The system 1400 can be incorporated into or used with any of the systems and devices described herein, such as the system 102 and/or user devices 104 of FIG. 1. The system 1400 can be used to perform any of the processes or methods described herein. The system 1400 can include a processor 1410, a memory 1420, a storage device 1430, and an input/output device 1440. Each of the components 1410, 1420, 1430 and 1440 can be interconnected using a system bus 1450. The processor 1410 can be configured to process instructions for execution within the system 1400. In some embodiments, the processor 1410 is a single-threaded processor. Alternatively, the processor 1410 can be a multi-threaded processor. The processor 1410 can be further configured to process instructions stored in the memory 1420 or on the storage device 1430, including receiving or sending information through the input/output device 1440. The memory 1420 can store information within the system 1400. In some embodiments, the memory 1420 is a computer-readable medium. Optionally, the memory 1420 can be a volatile memory unit or a non-volatile memory unit. The storage device 1430 can be capable of providing mass storage for the system 1400. In some embodiments, the storage device 1430 is a computer-readable medium. Optionally, the storage device 1430 can be a floppy disk device, a hard disk device, an optical disk device, a tape device, non-volatile solid-state memory, or any other type of storage device. The input/output device 1440 can be configured to provide input/output operations for the system 1400. In some embodiments, the input/output device 1440 can include a keyboard and/or pointing device. The input/output device 1440 can also include a display unit for displaying graphical user interfaces.

The systems and methods disclosed herein may be embodied in various forms including, for example, a data processor, such as a computer that also includes a database, digital electronic circuitry, firmware, software, or in combinations thereof. Moreover, the above-noted features and other aspects and principles of the present disclosed implementations may be implemented in various environments. Such environments and related applications may be specially constructed for performing the various processes and operations according to the embodiments disclosed herein, or they may include a general-purpose computer or computing platform selectively activated or reconfigured by code to provide the necessary functionality. The processes disclosed herein are not inherently related to any particular computer, network, architecture, environment, or other apparatus, and may be implemented by a suitable combination of hardware, software, and/or firmware. For example, various general-purpose machines may be used with programs written in accordance with teachings of the disclosed embodiments, or it may be more convenient to construct a specialized apparatus or system to perform the required methods and techniques.

The systems and methods disclosed herein may be implemented as a computer program product, i.e., a computer program tangibly embodied in an information carrier, e.g., in a machine readable storage device or in a propagated signal, for execution by, or to control the operation of, data processing apparatus, e.g., a programmable processor, a computer, or multiple computers. A computer program may be written in any form of programming language, including compiled or interpreted languages, and it may be deployed in any form, including as a stand-alone program or as a module, component, subroutine, or other unit suitable for use in a computing environment. A computer program may be deployed to be executed on one computer or on multiple computers at one site or distributed across multiple sites and interconnected by a communication network.

These computer programs, which may also be referred to programs, software, software applications, applications, components, or code, include machine instructions for a programmable processor, and may be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium may store such machine instructions non-transitorily, such as for example as would a non-transient solid state memory or a magnetic hard drive or any equivalent storage medium. The machine-readable medium may alternatively or additionally store such machine instructions in a transient manner, such as for example as would a processor cache or other random access memory associated with one or more physical processor cores.

To provide for interaction with a user, the subject matter described herein may be implemented on a computer having a display device, such as for example a cathode ray tube (CRT) or a liquid crystal display (LCD) monitor for displaying information to the user and a keyboard and a pointing device, such as for example a mouse or a trackball, by which the user may provide input to the computer. Other kinds of devices may be used to provide for interaction with a user as well. For example, feedback provided to the user may be any form of sensory feedback, such as for example visual feedback, auditory feedback, or tactile feedback; and input from the user may be received in any form, including, but not limited to, acoustic, speech, or tactile input.

The technology described herein may be implemented in a computing system that includes a back-end component, such as for example one or more data servers, or that includes a middleware component, such as for example one or more application servers, or that includes a front-end component, such as for example one or more client computers having a graphical user interface or a Web browser through which a user may interact with an embodiment of the technology described herein, or any combination of such back-end, middleware, or front-end components. The components of the system may be interconnected by any form or medium of digital data communication, such as for example a communication network. Examples of communication networks include, but are not limited to, a LAN, a WAN, and the Internet.

The computing system may include clients and servers. A client and server are generally, but not exclusively, remote from each other and typically interact through a communication network. The relationship of client and server arises by virtue of computer programs running on the respective computers and having a client-server relationship to each other.

### VII. CONCLUSION

The embodiments set forth in the foregoing description do not represent all embodiments consistent with the subject matter described herein. Instead, they are merely some examples consistent with aspects related to the described subject matter. Although a few variations have been described in detail above, other modifications or additions are possible. In particular, further features and/or variations may be provided in addition to those set forth herein. For example, the embodiments described above may be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other embodiments may be within the scope of the following claims.

Furthermore, the skilled artisan will recognize the interchangeability of various features from different embodiments disclosed herein and disclosed in U.S. Pat. Nos. 9,008,745; 9,182,368; 10,173,042; U.S. Patent Application Publication No. 2017/0251958; U.S. Patent Application Publication No. 2018/0140235; U.S. Patent Application Publication No. 2016/0029931; U.S. Patent Application Publication No. 2016/0029966; U.S. Patent Application Publication No. 2017/0128009; U.S. Provisional Application No. 62/855,194; U.S. Provisional Application No. 62/854,088; US App. Provisional Application No. 62/970,282; and International Publication No. WO 2020/051101, which are all hereby incorporated by reference in their entireties. These technologies can be used with, incorporated into, and/or combined with any of the systems, methods, devices, features, and components disclosed herein. For example, biomonitoring and forecasting systems, biosensors, user devices, methods for forecasting health parameters, manufacturing methods, etc., can be incorporated into or used with the technology disclosed herein. All of these applications are incorporated herein by reference in their entireties. Similarly, the various features and acts discussed above, as well as other known equivalents for each such feature or act, can be mixed and matched by one of ordinary skill in this art to perform methods in accordance with principles described herein.

As used herein, the term “user” may refer to any entity including a person or a computer.

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

As used herein and in the appended claims, the singular forms “a,” “an,” and “the” include plural references unless the context clearly dictates otherwise.

As used herein, the phrase “and/or” as in “A and/or B” refers to A alone, B alone, and A and B.

Although ordinal numbers such as first, second, and the like can, in some situations, relate to an order; as used in this document ordinal numbers do not necessarily imply an order. For example, ordinal numbers can be merely used to distinguish one item from another. For example, to distinguish a first event from a second event, but need not imply any chronological ordering or a fixed reference system (such that a first event in one paragraph of the description can be different from a first event in another paragraph of the description).

From the foregoing, it will be appreciated that specific embodiments of the invention have been described herein for purposes of illustration, but that various modifications may be made without deviating from the scope of the invention. Accordingly, the invention is not limited except as by the appended claims.

1. A device for monitoring a user's health, the device comprising:
a patch comprising:
a substrate configured to couple to the user's skin; and
an array of microneedles carried by the substrate, the array of microneedles configured to access interstitial fluid in the user's skin and generate a first electrical signal indicative of at least one analyte in the interstitial fluid;
a pod configured to releasably couple to the patch, the pod comprising:
at least one sensor configured to generate a second electrical signal indicative of a physiological parameter of the user;
a processor;
a memory operably coupled to the processor and storing instructions that, when executed by the processor, cause the processor to:
receive and process the first electrical signal from the array of microneedles to generate a first health measurement for the user, and
receive and process the second electrical signal from the at least one sensor to generate a second health measurement for the user; and
a communication unit configured to transmit the first and second health measurements to a remote device.

2. The device of claim 1 wherein the at least one analyte includes one or more of the following: glucose, lactic acid, alcohol, creatine, potassium, sodium, urea, blood urea nitrogen, ketones, or bicarbonate.

3. The device of claim 1 wherein the first health measurement includes a concentration of the at least one analyte.

4. The device of claim 1 wherein the array of microneedles is configured to detect two or more different analytes in the interstitial fluid.

5. The device of claim 4 wherein the array of microneedles includes at least one microneedle configured to detect the two or more different analytes.

6. The device of claim 1 wherein the array of microneedles is a first array of microneedles, and the at least one analyte is a first analyte, and the patch further comprises a second array of microneedles configured to detect a second, different analyte.

7. The device of claim 1 wherein the array of microneedles is a first array of microneedles configured as a working electrode, and the patch further comprises:
a second array of microneedles configured as a reference electrode; and
a third array of microneedles configured as a counter electrode.

8. The device of claim 1 wherein the microneedles are configured to access an epidermal layer of the user's skin.

9-10. (canceled)

11. The device of claim 1 wherein the substrate includes an adhesive region configured to releasably couple to the user's skin, and wherein the adhesive region at least partially surrounds the array of microneedles.

12. (canceled)

13. The device of claim 1 wherein:
the array of microneedles is coupled to a lower surface of the substrate; and
the patch includes an annular housing on an upper surface of the substrate, the annular housing configured to receive and couple to the pod.

14. The device of claim 1 wherein the patch includes a set of first electrical contacts on an upper surface of the substrate, the set of first electrical contacts being electrically coupled to the array of microneedles.

15. The device of claim 14 wherein:
the pod includes a set of second electrical contacts on a lower surface of the pod, the set of second electrical contacts being electrically coupled to the processor; and
when the pod is coupled to the patch, the processor is electrically coupled to the array of microneedles via the first and second electrical contacts.

16. The device of claim 1 wherein the array of microneedles is further configured to generate a third electrical signal indicative of bioimpedance of the user's skin and wherein the instructions further cause the processor to receive and process the third electrical signal to determine penetration of the microneedles into the user's skin.

17. (canceled)

18. The device of claim 1 wherein the patch further includes a temperature sensor carried by the substrate and configured to generate a third electrical signal indicative of the user's body temperature, and wherein the instructions further cause the processor to:
receive and process the third electrical signal to determine the user's body temperature; and
adjust processing of one or more of the first and second electrical signals, based on the user's body temperature.

19-20. (canceled)

21. The device of claim 1 wherein the at least one sensor includes a photoplethysmography (PPG) sensor, and the physiological parameter includes one or more of an oxygen level or a heart rate of the user.

22. The device of claim 21 wherein:
the pod includes a protrusion housing the PPG sensor, and a window in the protrusion forming an optical path to the PPG sensor; and
the substrate of the patch includes an aperture configured to receive the protrusion, such that when the substrate is coupled to the user's skin, the window of the protrusion is placed in direct contact with the user's skin.

23. The device of claim 1 wherein the at least one sensor includes at least one of an electrocardiogram (ECG) electrode and a motion sensor.

24-27. (canceled)

28. The device of claim 1 wherein the pod is configured to be reusable and the patch is configured to be disposable, and wherein the pod is configured to be sequentially coupled to a plurality of disposable patches.

29. (canceled)

30. The device of claim 28 wherein at least some of the disposable patches are configured to detect different analytes.

31. The device of claim 28 wherein:
each patch is associated with a respective identifier; and
when the pod is coupled to an individual patch, the instructions cause the processor to:
detect the respective identifier of the individual patch; and
adjust a signal processing parameter based on the detected identifier.

32-70. (canceled)

71. A device for monitoring a user's health, the device comprising:
an adhesive patch coupleable to a user's skin including an array of microneedles configured to access interstitial fluid in the user's skin and generate a first electrical signal indicative of at least one analyte in the interstitial fluid; and
a pod configured to releasably couple to the patch, the pod comprising:
at least one sensor configured to analyze the user's tissue to generate a second electrical signal indicative of a physiological parameter of the user when the adhesive patch is coupled to the user's skin;
a power source configured to output power to the adhesive patch to cause the array of microneedles to generate the first electrical signal;
at least one processor configured to:
receive and process the first electrical signal from the array of microneedles to generate first health data for the user, and
receive and process the second electrical signal from the at least one sensor to generate second health data for the user; and
a communication unit configured to wirelessly transmit the first and second health data to a remote device.

72. The device of claim 71 wherein the pod includes memory storing instructions executable by the at least one processor to cause the at least one processor to detect coupling of the pod to the adhesive patch.

73. The device of claim 71 wherein the pod includes memory storing instructions executable by the at least one processor to cause the at least one processor to:
detect the adhesive patch,
determine detection information for the adhesive patch, and
process the first electrical signal from the array of microneedles based on the detection information.

74. The device of claim 71 wherein the pod includes memory storing instructions executable by the at least one processor to cause the at least one processor to determine when the pod is coupled to another patch.

75. The device of claim 71 wherein the pod includes memory storing a plurality of algorithms and instructions executable by the at least one processor to cause the at least one processor to:
identify the adhesive patch,
select at least one of the algorithms based on the identification, and
process the first electrical signal using the selected at least one algorithm.

76. The device of claim 75 wherein the pod includes memory storing a plurality of algorithms and instructions executable by the at least one processor to cause the at least one processor to generate a set of analyte values using the selected at least one algorithm.

77. The device of claim 71 wherein the pod includes memory storing a plurality of algorithms and instructions executable by the at least one processor to cause the at least one processor to wirelessly retrieve programming to add a new patch functionality previously not provided by the pod.

78. The device of claim 71 wherein the pod includes memory storing a plurality of algorithms and instructions executable by the at least one processor to cause the at least one processor to determine newly available functionality of the adhesive patch.

79-85. (canceled)

86. A device for monitoring a user, the device comprising:
a patch configured to couple to the user's skin and including means for accessing interstitial fluid in the user's skin and generating a first electrical signal indicative of at least one analyte in the interstitial fluid;
a pod configured to releasably couple to the patch, the pod comprising:
means for generating a second electrical signal indicative of at least one physiological parameter of the user; and
processing means for:
processing the first electrical signal to generate a first health measurement for the user, and
processing the second electrical signal to generate a second health measurement for the user; and
communication means for transmitting the first and second health measurements to a remote device.

87. The device of claim 86 wherein the means for accessing interstitial fluid in the user's skin and generating the first electrical signal includes one or more microneedles.

88. The device of claim 86 wherein the means for accessing interstitial fluid in the user's skin and generating the first electrical signal includes one or more arrays of microneedles configured to detect two or more different analytes in the interstitial fluid.

89. The device of claim 86 wherein the means for generating the second electrical signal includes at least one optical sensor.

90. The device of claim 89 wherein the at least one optical sensor includes a photoplethysmography (PPG) sensor, and the physiological parameter includes one or more of an oxygen level and/or a heart rate of the user.

91. The device of claim 86 wherein the means for generating the second electrical signal includes an electrocardiogram (ECG) electrode.

92. The device of claim 86 wherein the means for generating the second electrical signal includes one or more motion sensors, accelerometers, and/or gyroscopes.

93-95. (canceled)
